SEQUENCE LISTING (122 sequences)

SEQ ID NO: 1 - DNA, 24 bp, Artificial Sequence
  Synthetic oligonucleotide
tgtcccaggtggcttacagatgaa

SEQ ID NO: 2 - DNA, 24 bp, Artificial Sequence
  Synthetic oligonucleotide
ggtgtgccaaagttgccaatacac

SEQ ID NO: 3 - DNA, 23 bp, Artificial Sequence
  Synthetic oligonucleotide
agcccaagtgacaagggacaact

SEQ ID NO: 4 - DNA, 24 bp, Artificial Sequence
  Synthetic oligonucleotide
aaggaaccacttcgaaggtgctga

SEQ ID NO: 5 - DNA, 20 bp, Artificial Sequence
  Synthetic oligonucleotide
ctgtgctcctggaactgaaa

SEQ ID NO: 6 - DNA, 21 bp, Artificial Sequence
  Synthetic oligonucleotide
tgggagctagagtcagagatg

SEQ ID NO: 7 - DNA, 19 bp, Artificial Sequence
  Synthetic oligonucleotide
ctgcgtcttcacggagatt

SEQ ID NO: 8 - DNA, 19 bp, Artificial Sequence
  Synthetic oligonucleotide
ggtgaaggccatgtaccag

SEQ ID NO: 9 - DNA, 22 bp, Artificial Sequence
  Synthetic oligonucleotide
cccactacaatgtgttcgtaga

SEQ ID NO: 10 - DNA, 21 bp, Artificial Sequence
  Synthetic oligonucleotide
tgcatgttattgtcggctttg

SEQ ID NO: 11 - DNA, 20 bp, Artificial Sequence
  Synthetic oligonucleotide
gtttgtctccggattcccaa

SEQ ID NO: 12 - DNA, 21 bp, Artificial Sequence
  Synthetic oligonucleotide
caggttctggtcgtcattctc

SEQ ID NO: 13 - DNA, 24 bp, Artificial Sequence
  Synthetic oligonucleotide
caacatgttcgagaacggcagctt

SEQ ID NO: 14 - DNA, 24 bp, Artificial Sequence
  Synthetic oligonucleotide
cgctcttgatcaccaccttcttct

SEQ ID NO: 15 - DNA, 24 bp, Artificial Sequence
  Synthetic oligonucleotide
cgaatgacggcatctgtttgccat

SEQ ID NO: 16 - DNA, 24 bp, Artificial Sequence
  Synthetic oligonucleotide
atttggtattagggatgcagggcg

SEQ ID NO: 17 - DNA, 22 bp, Artificial Sequence
  Synthetic oligonucleotide
tctccaataggctctcctacaa

SEQ ID NO: 18 - DNA, 22 bp, Artificial Sequence
  Synthetic oligonucleotide
taagtttctgcaggtgcatact

SEQ ID NO: 19 - DNA, 19 bp, Artificial Sequence
  Synthetic oligonucleotide
acccggactcctacaacat

SEQ ID NO: 20 - DNA, 20 bp, Artificial Sequence
  Synthetic oligonucleotide
tccttctcctccttgtcctt

SEQ ID NO: 21 - DNA, 20 bp, Artificial Sequence
  Synthetic oligonucleotide
gagtgagagagtcgtgtttc

SEQ ID NO: 22 - DNA, 20 bp, Artificial Sequence
  Synthetic oligonucleotide
acttctgtccctttcctttc

SEQ ID NO: 23 - DNA, 22 bp, Artificial Sequence
  Synthetic oligonucleotide
ccacctcctacttgcctatcta

SEQ ID NO: 24 - DNA, 18 bp, Artificial Sequence
  Synthetic oligonucleotide
gctggttgacggacactg

SEQ ID NO: 25 - DNA, 24 bp, Artificial Sequence
  Synthetic oligonucleotide
gaaggtggagctgcattggtttga

SEQ ID NO: 26 - DNA, 24 bp, Artificial Sequence
  Synthetic oligonucleotide
taaaccagctacaggacaggccaa

SEQ ID NO: 27 - DNA, 24 bp, Artificial Sequence
  Synthetic oligonucleotide
tgctacaaccgtgccagtgactat

SEQ ID NO: 28 - DNA, 24 bp, Artificial Sequence
  Synthetic oligonucleotide
agtggtctttcaggtgtgagcagt

SEQ ID NO: 29 - DNA, 20 bp, Artificial Sequence
  Synthetic oligonucleotide
agaaggagacggaggctatt

SEQ ID NO: 30 - DNA, 20 bp, Artificial Sequence
  Synthetic oligonucleotide
ggtctgctcgttcccataag

SEQ ID NO: 31 - DNA, 24 bp, Artificial Sequence
  Synthetic oligonucleotide
tgtccctaccttgtctgttagcca

SEQ ID NO: 32 - DNA, 24 bp, Artificial Sequence
  Synthetic oligonucleotide
attggaacatggcctctggatgga

SEQ ID NO: 33 - DNA, 22 bp, Artificial Sequence
  Synthetic oligonucleotide
gaaagttggagagtacggctac

SEQ ID NO: 34 - DNA, 22 bp, Artificial Sequence
  Synthetic oligonucleotide
tgttgaagtggaactccttctc

SEQ ID NO: 35 - DNA, 20 bp, Artificial Sequence
  Synthetic oligonucleotide
gcccgctacttgtttcttct

SEQ ID NO: 36 - DNA, 21 bp, Artificial Sequence
  Synthetic oligonucleotide
gtgaatgtagacacggtcagg

SEQ ID NO: 37 - DNA, 24 bp, Artificial Sequence
  Synthetic oligonucleotide
acaaagtgaaggtgacgggcctta

SEQ ID NO: 38 - DNA, 24 bp, Artificial Sequence
  Synthetic oligonucleotide
atctgtgatcgtcggcaggtacaa

SEQ ID NO: 39 - DNA, 20 bp, Artificial Sequence
  Synthetic oligonucleotide
gtgaagaagaacgcgaaggt

SEQ ID NO: 40 - DNA, 19 bp, Artificial Sequence
  Synthetic oligonucleotide
tggtgacgatcatctcggt

SEQ ID NO: 41 - DNA, 22 bp, Artificial Sequence
  Synthetic oligonucleotide
tgtggaaatggatactgacaca

SEQ ID NO: 42 - DNA, 20 bp, Artificial Sequence
  Synthetic oligonucleotide
gacggaagacaccaagaaca

SEQ ID NO: 43 - DNA, 19 bp, Artificial Sequence
  Synthetic oligonucleotide
gctggagttaaaccagtga

SEQ ID NO: 44 - DNA, 17 bp, Artificial Sequence
  Synthetic oligonucleotide
gatgcgtagcaccagtc

SEQ ID NO: 45 - DNA, 18 bp, Artificial Sequence
  Synthetic oligonucleotide
gcgtgaatgttccctctt

SEQ ID NO: 46 - DNA, 21 bp, Artificial Sequence
  Synthetic oligonucleotide
ctagtgcccactgtctttatc

SEQ ID NO: 47 - DNA, 21 bp, Artificial Sequence
  Synthetic oligonucleotide
tgatgtagcccatgtggaaag

SEQ ID NO: 48 - DNA, 21 bp, Artificial Sequence
  Synthetic oligonucleotide
gctgccggacagtaacatatc

SEQ ID NO: 49 - DNA, 24 bp, Artificial Sequence
  Synthetic oligonucleotide
tttgccaagttccattgtgccagg

SEQ ID NO: 50 - DNA, 24 bp, Artificial Sequence
  Synthetic oligonucleotide
tggtggagtcactggaaagcagaa

SEQ ID NO: 51 - DNA, 24 bp, Artificial Sequence
  Synthetic oligonucleotide
agggaactgaacattccagagcgt

SEQ ID NO: 52 - DNA, 22 bp, Artificial Sequence
  Synthetic oligonucleotide
tttgtacaccaagcacctattt

SEQ ID NO: 53 - DNA, 21 bp, Artificial Sequence
  Synthetic oligonucleotide
aggaatttgctcgtccatctc

SEQ ID NO: 54 - DNA, 18 bp, Artificial Sequence
  Synthetic oligonucleotide
aagaccgaggccgaattg

SEQ ID NO: 55 - DNA, 24 bp, Artificial Sequence
  Synthetic oligonucleotide
agactgaaagcaaagcagcactcc

SEQ ID NO: 56 - DNA, 24 bp, Artificial Sequence
  Synthetic oligonucleotide
atactggcaagcactcaggttgga

SEQ ID NO: 57 - DNA, 24 bp, Artificial Sequence
  Synthetic oligonucleotide
acgatgctactttcttgcacacgc

SEQ ID NO: 58 - DNA, 26 bp, Artificial Sequence
  Synthetic oligonucleotide
accctcccggctctgtacactattta

SEQ ID NO: 59 - DNA, 19 bp, Artificial Sequence
  Synthetic oligonucleotide
tccgcgagtaccaactcta

SEQ ID NO: 60 - DNA, 19 bp, Artificial Sequence
  Synthetic oligonucleotide
caaacttgttgccgtcctc

SEQ ID NO: 61 - DNA, 24 bp, Artificial Sequence
  Synthetic oligonucleotide
tcaggcagtccttgaggatagaca

SEQ ID NO: 62 - DNA, 24 bp, Artificial Sequence
  Synthetic oligonucleotide
aagcagtctgtgtagtgtgtgggt

SEQ ID NO: 63 - DNA, 21 bp, Artificial Sequence
  Synthetic oligonucleotide
gagaacagcgagctcaagtat

SEQ ID NO: 64 - DNA, 17 bp, Artificial Sequence
  Synthetic oligonucleotide
cacggaggacgtgactt

SEQ ID NO: 65 - DNA, 23 bp, Artificial Sequence
  Synthetic oligonucleotide
cacctgctgttctcttgtttatg

SEQ ID NO: 66 - DNA, 23 bp, Artificial Sequence
  Synthetic oligonucleotide
cagtaatagtgaggcaggagaac

SEQ ID NO: 67 - DNA, 18 bp, Artificial Sequence
  Synthetic oligonucleotide
tggcaaccacaggtcatt

SEQ ID NO: 68 - DNA, 21 bp, Artificial Sequence
  Synthetic oligonucleotide
ctggtttcatgtcatcttcgg

SEQ ID NO: 69 - DNA, 22 bp, Artificial Sequence
  Synthetic oligonucleotide
ctgacctcctccctcaactatt

SEQ ID NO: 70 - DNA, 23 bp, Artificial Sequence
  Synthetic oligonucleotide
gggtattctggttctccacattc

SEQ ID NO: 71 - DNA, 22 bp, Artificial Sequence
  Synthetic oligonucleotide
gatcaagagactcccgaagaag

SEQ ID NO: 72 - DNA, 22 bp, Artificial Sequence
  Synthetic oligonucleotide
gtagttcagagcaggcgataag

SEQ ID NO: 73 - DNA, 19 bp, Artificial Sequence
  Synthetic oligonucleotide
gctgctacttgtccggttt

SEQ ID NO: 74 - DNA, 17 bp, Artificial Sequence
  Synthetic oligonucleotide
cgcagatagccgtgcag

SEQ ID NO: 75 - DNA, 19 bp, Artificial Sequence
  Synthetic oligonucleotide
caactggctccagtcactc

SEQ ID NO: 76 - DNA, 21 bp, Artificial Sequence
  Synthetic oligonucleotide
gcaggtactcctcgaaattct

SEQ ID NO: 77 - DNA, 18 bp, Artificial Sequence
  Synthetic oligonucleotide
aacgccatgctgaggaaa

SEQ ID NO: 78 - DNA, 20 bp, Artificial Sequence
  Synthetic oligonucleotide
cgtggtggatgtcttgatgt

SEQ ID NO: 79 - DNA, 19 bp, Artificial Sequence
  Synthetic oligonucleotide
gccagaaaccagccatagt

SEQ ID NO: 80 - DNA, 22 bp, Artificial Sequence
  Synthetic oligonucleotide
cataggtttgctccctaccttc

SEQ ID NO: 81 - DNA, 21 bp, Artificial Sequence
  Synthetic oligonucleotide
tgtacttcactgtggtgatgg

SEQ ID NO: 82 - DNA, 21 bp, Artificial Sequence
  Synthetic oligonucleotide
gatggcctgacgattcttgta

SEQ ID NO: 83 - DNA, 21 bp, Artificial Sequence
  Synthetic oligonucleotide
ccaagggctggaatgttatct

SEQ ID NO: 84 - DNA, 22 bp, Artificial Sequence
  Synthetic oligonucleotide
ggagattgtccacaggatacac

SEQ ID NO: 85 - DNA, 20 bp, Artificial Sequence
  Synthetic oligonucleotide
cctgggaacgcctcaaataa

SEQ ID NO: 86 - DNA, 22 bp, Artificial Sequence
  Synthetic oligonucleotide
gaatggccaagtttacccaaag

SEQ ID NO: 87 - DNA, 21 bp, Artificial Sequence
  Synthetic oligonucleotide
cagacctaccacaacgagatg

SEQ ID NO: 88 - DNA, 20 bp, Artificial Sequence
  Synthetic oligonucleotide
acacagtgttctgggtgaag

SEQ ID NO: 89 - DNA, 20 bp, Artificial Sequence
  Synthetic oligonucleotide
tacctgtccggctacatctc

SEQ ID NO: 90 - DNA, 22 bp, Artificial Sequence
  Synthetic oligonucleotide
cagatgttctcgatgcccataa

SEQ ID NO: 91 - DNA, 18 bp, Artificial Sequence
  Synthetic oligonucleotide
tgccgtcgtttggcttta

SEQ ID NO: 92 - DNA, 22 bp, Artificial Sequence
  Synthetic oligonucleotide
cttgagtacgctctcgttcttg

SEQ ID NO: 93 - DNA, 21 bp, Artificial Sequence
  Synthetic oligonucleotide
cggacatcaaggctctgtatc

SEQ ID NO: 94 - DNA, 22 bp, Artificial Sequence
  Synthetic oligonucleotide
catctcctggaagttctggttc

SEQ ID NO: 95 - DNA, 24 bp, Artificial Sequence
  Synthetic oligonucleotide
ttggactcctgggaacatggacaa

SEQ ID NO: 96 - DNA, 24 bp, Artificial Sequence
  Synthetic oligonucleotide
atgcttcagggtatctggcctctt

SEQ ID NO: 97 - DNA, 24 bp, Artificial Sequence
  Synthetic oligonucleotide
tgggctagacacgggagaatactt

SEQ ID NO: 98 - DNA, 24 bp, Artificial Sequence
  Synthetic oligonucleotide
aagatgtagagccgtttccgctca

SEQ ID NO: 99 - DNA, 20 bp, Artificial Sequence
  Synthetic oligonucleotide
gactgtacacctgccacatc

SEQ ID NO: 100 - DNA, 18 bp, Artificial Sequence
  Synthetic oligonucleotide
caggaaggagcgcatgtt

SEQ ID NO: 101 - DNA, 19 bp, Artificial Sequence
  Synthetic oligonucleotide
ggttcaccacctgaaccac

SEQ ID NO: 102 - DNA, 19 bp, Artificial Sequence
  Synthetic oligonucleotide
tgacagcggatcccatact

SEQ ID NO: 103 - DNA, 21 bp, Artificial Sequence
  Synthetic oligonucleotide
gctggtcagggattctcatac

SEQ ID NO: 104 - DNA, 21 bp, Artificial Sequence
  Synthetic oligonucleotide
ctgctgatcagagccatactg

SEQ ID NO: 105 - DNA, 21 bp, Artificial Sequence
  Synthetic oligonucleotide
gctagccatcatcaccaagat

SEQ ID NO: 106 - DNA, 22 bp, Artificial Sequence
  Synthetic oligonucleotide
ccaggtcatcttgttctccttc

SEQ ID NO: 107 - DNA, 21 bp, Artificial Sequence
  Synthetic oligonucleotide
gtccaaccaccagagaaagag

SEQ ID NO: 108 - DNA, 21 bp, Artificial Sequence
  Synthetic oligonucleotide
gcagcagacccataagtgtta

SEQ ID NO: 109 - DNA, 18 bp, Artificial Sequence
  Synthetic oligonucleotide
gctgatccgctccttcag

SEQ ID NO: 110 - DNA, 21 bp, Artificial Sequence
  Synthetic oligonucleotide
tggaagagcttctgtttctcg

SEQ ID NO: 111 - DNA, 22 bp, Artificial Sequence
  Synthetic oligonucleotide
accattgaaatgcctcaacaag

SEQ ID NO: 112 - DNA, 23 bp, Artificial Sequence
  Synthetic oligonucleotide
acgatgatacagatcagcaagag

SEQ ID NO: 113 - DNA, 20 bp, Artificial Sequence
  Synthetic oligonucleotide
gctcctgggtatcatcttct

SEQ ID NO: 114 - DNA, 20 bp, Artificial Sequence
  Synthetic oligonucleotide
gaggaatgggtgttgctatg

SEQ ID NO: 115 - DNA, 22 bp, Artificial Sequence
  Synthetic oligonucleotide
ctgttcttgtaacagcccagaa

SEQ ID NO: 116 - DNA, 20 bp, Artificial Sequence
  Synthetic oligonucleotide
tagcaagatggtccctagca

SEQ ID NO: 117 - DNA, 24 bp, Artificial Sequence
  Synthetic oligonucleotide
ttaaccttgtccgtctgcctgagt

SEQ ID NO: 118 - DNA, 24 bp, Artificial Sequence
  Synthetic oligonucleotide
gtaatgggctttggcctttgcact

SEQ ID NO: 119 - DNA, 19 bp, Artificial Sequence
  Synthetic oligonucleotide
tctttgtcgcgatggtagc

SEQ ID NO: 120 - DNA, 23 bp, Artificial Sequence
  Synthetic oligonucleotide
ggttcttgatagcgttggaattg

SEQ ID NO: 121 - DNA, 24 bp, Artificial Sequence
  Synthetic oligonucleotide
tgagttgctcataccgtgctgcta

SEQ ID NO: 122 - DNA, 24 bp, Artificial Sequence
  Synthetic oligonucleotide
ccctcaaaccaacttgtcaacagc

(19) United States
(12) Patent Application Publication
(10) Pub. No.: US 2025/0320458 A1
(43) Pub. Date: Oct. 16, 2025

(54) CARDIOMYOCYTE SUBTYPES AND METHODS OF MAKING AND USING

(71) Applicant: University Health Network, Toronto (CA)

(72) Inventors: Gordon M. Keller, Toronto (CA); Donghe YANG, Toronto (CA)

(21) Appl. No.: 18/569,063

(22) PCT Filed: Jun. 10, 2022
(86) PCT No.: PCT/IB2022/055425

§ 371 Date: Dec. 11, 2023

Related U.S. Application Data
(60) Provisional application No. 63/209,703, filed on Jun. 11, 2021.

(51) Int. Cl. C12N 5/077

(52) U.S. Cl. CPC C12N 5/0657; C12N 2500/02; C12N 2501/115; C12N 2501/155; C12N 2501/165; C12N 2501/998; C12N 2506/45

(57) ABSTRACT

Cardiomyocyte subtypes, including first heart field (FHF) and second heart field (SHF) (e.g., anterior second heart field (aSHF) and posterior second heart field (pSHF)) cells, and methods of making and using such cells, are described.

### CROSS-REFERENCE TO RELATED APPLICATIONS

This application claims the benefit of U.S. Provisional Application Ser. No. 63/209,703, filed Jun. 11, 2022. The disclosure of the prior application is considered part of (and is incorporated by reference in) the disclosure of this application.

### TECHNICAL FIELD

This disclosure generally relates to several different cardiomyoctye subtypes as well as methods of making and using such cardiomyocyte subtypes.

### BACKGROUND

The adult heart is made-up of different cardiomyocyte subtypes that include left and right ventricular and atrial cardiomyocytes that form the working myocardium, the sinoatrial and atrioventricular nodal cells that represent the pacemakers and the outflow and inflow tract cells that connect the heart to the vasculature. The ability to differentiate human pluripotent stem cells (hPSCs) into different cardiovascular lineages has opened new and exciting avenues to study the earliest stages of human heart development, to generate models of heart disease and to create new therapies to treat some of the most devastating and debilitating of these diseases. As different cardiovascular diseases target different regions of the heart, the therapeutic applications of these models are entirely dependent on the ability to generate appropriate cell types from hPSCs.

This disclosure describes a number of different cardiomyocyte subtypes, as well as methods of making and using such cardiomyocyte subtypes.

### SUMMARY

This disclosure provides a comprehensive landscape of human embryonic cardiogenesis, which can be used to model a broad spectrum of congenital heart diseases and chamber-specific cardiomyopathies with hPSCs. From these analyses, both novel and species-conserved markers were identified that provide a molecular signature for the different stages of human FHF, pSHF and aSHF development. Through the staged manipulation of signaling pathways identified from the scRNA-seq analysis, myocyte populations can be generated that display molecular characteristics of right ventricular cardiomyocytes (RVCMs), left ventricular cardiomyocytes (LVCMs), atrial cardiomyocytes (ACMs), atrioventricular canal cardiomyocytes (AVCCMs), sinus venosus cardiomyocytes (SVCMs), inflow tract cardiomyocytes (IFTCMs) and outflow tract cardiomyocytes (OFTCMs). Collectively, this disclosure provides new insights into human cardiac lineage development that enables the design of improved lineage-specific differentiation protocols as well as access to different cardiomyocyte subtypes for modeling chamber-specific cardiovascular diseases and congenital heart defects and for establishing novel therapeutic approaches to treat them.

In one aspect, methods of making first heart field (FHF) mesoderm cells, anterior second heart field (aSHF) mesoderm cells and/or posterior second heart field (pSHF) mesoderm cells are provided. Such methods typically include: culturing pluripotent stem cells (PSCs) in the presence of an appropriate amount of BMP4 and Activin A for about 1 to about 3 days (e.g., about 2 days); thereby producing FHF mesoderm cells, aSHF mesoderm cells, and/or pSHF mesoderm cells, wherein the FHF mesoderm cells are MESP1+, CXCR4−/low, GYPB+, CD1Dlow, TDGF1+, LHX1+, PITX2+, and GSC+, wherein the aSHF mesoderm cells are MESP1+, CXCR4+, ALDH1A2−, CD1Dlow, PHLDA1+, PCDH19+, FOXC2+, TWIST1+, and FOXC1+, and wherein the pSHF mesoderm cells are MESP1+, CXCR4−, ALDHIA2+, CD1Dhigh, HOXA1+, HOXB1+,

In one aspect, FHF mesoderm cells, aSHF mesoderm cells and/or pSHF mesoderm cells made by the methods described herein are provided.

In another aspect, methods of screening drugs are provided. Such methods typically include: contacting the FHF mesoderm cells, the aSHF mesoderm cells described herein with a test compound; and determining the effect of the compound on the differentiation of the FHF mesoderm cells, the aSHF mesoderm cells, or the pSHF mesoderm cells into progenitor cells and, optionally, into cardiomyocytes.

In another aspect, methods of making first heart field (FHF) mesoderm cells are provided. Such methods typically include: culturing pluripotent stem cells (PSCs) in the presence of an appropriate amount of BMP4 and Activin A for about 1 to about 3 days (e.g., about 2 days), thereby producing FHF mesoderm cells, wherein the FHF mesoderm cells are MESP1+, CXCR4−/low, GYPB+, CD1Dlow, TDGF1+, LHX1+, PITX2+, and GSC+.

In one aspect, FHF mesoderm cells made by the methods described herein are provided.

In still another aspect, methods of making first heart field (FHF) progenitor cells are provided. Such methods typically include: culturing the FHF mesoderm cells as described herein in the presence of an appropriate amount of IWP2 and VEGF for a period of about 1 to about 3 days (e.g., about 2 days), thereby producing FHF progenitor cells, wherein the FHF progenitor cells are ALDH1 A2−, HAND1, TBX5, HCN4, MYH6, LBH.

In another aspect, FHF progenitor cells made by the methods described herein are provided.

In yet another aspect, methods of making first heart field (FHF) cardiomyocytes are provided. Such methods typically include: culturing the FHF progenitor cells as described herein in base media for about 18 to about 22 days (e.g., about 20 days), thereby producing FHF cardiomyocytes, wherein the FHF cardiomyocytes comprise a first population of left ventricular cardiomyocytes (LVCMs) that are GJA1+, HAND1+, TMEM88+, and TBX5+ and a second population of atrioventricular canal cardiomyocytes (AVCCMs) that are BMP2+, TBX2+, RSPO3+, and MSX2+.

In yet another aspect, FHF cardiomyocytes made by the methods described herein are provided. In some embodiments, the FHF mesoderm cells and the FHF progenitor cells can be differentiated into left ventricular cardiomyocytes (LVCMs) and atrioventricular canal cardiomyocytes (AVCCMs).

In one aspect, methods of repairing damaged cardiac tissue are provided. Such methods typically include introducing the FHF mesoderm cells, the FHF progenitor cells, and/or the FHF cardiomyocytes described herein into damaged cardiac tissue. In some embodiments, the damaged cardiac tissue comprises ventricular myocardium.

In another aspect, methods of making anterior second heart field (aSHF) mesoderm cells are provided. Such methods typically include: culturing pluripotent stem cells (PSCs) in the presence of an appropriate amount of BMP4 and Activin-A for about 1 to about 3 days (e.g., about 2 days), thereby producing aSHF mesoderm cells, wherein the aSHF mesoderm cells are MESP1+, CXCR4+, ALDHIA2−, CD1Dlow, PHLDA1+, PCDH19+, FOXC2+, TWIST1+, and FOXC1+.

In another aspect, aSHF mesoderm cells made by the methods described herein are provided.

In still another aspect, methods of making anterior second heart field (aSHF) progenitor cells are provided. Such methods typically include: culturing the aSHF mesoderm cells as described herein as an (intact) embryoid body (EB) or isolated day 4 CXCR+ ALDH− mesoderm cells in the presence of an appropriate amount of IWP2 and VEGF for about 1 to about 3 days (e.g., about 1 to about 2 days), thereby producing aSHF progenitor cells, wherein the aSHF progenitor cells are ALDH1A2+, JAG1+, FGF10+, FGF8+, WNT5A+, and PHLDA1+.

In another aspect, aSHF progenitor cells made by the methods described herein are provided.

In yet another aspect, methods of making anterior second heart field (aSHF) cardiomyocytes are provided. Such methods typically include: culturing the aSHF progenitor cells as described herein or isolated day 4 CXCR+ALDH− mesoderm cells in the presence of an appropriate amount of BMP4 and RA for about 3 days and then in backbone media for about 12 to about 15 days, thereby making aSHF cardiomyocytes, wherein the aSHF cardiomyocytes comprise a first population of right ventricular cardiomyocytes (RVCMs) that are IRX1+, IRX2+, and NPPB+ and a second population of outflow tract cardiomyocytes (OFTCM) that are SEMA3C+, HAND2+, and FHL1+.

In another aspect, aSHF cardiomyocytes made by the methods described herein are provided. In some embodiments, the aSHF mesoderm and/or progenitors can be differentiated into right ventricular cardiomyocytes (RVCMs) and outflow tract (OFT) cardiomyocytes.

In still another aspect, methods of modeling chamber-specific diseases such as arrhythmogenic right ventricular cardiomyopathy (ARVC) and OFT defects are provided. Such methods typically include culturing the aSHF mesoderm cells, the aSHF progenitor cells, and/or the aSHF cardiomyocytes described herein under a variety of culture conditions and evaluating their characteristics.

In another aspect, methods of making posterior second heart field (pSHF) mesoderm cells are provided. Such methods typically include: culturing pluripotent stem cells (PSCs) in the presence of an appropriate amount of BMP4 and Activin A for about 1 to about 3 days (e.g., about 2 days), thereby producing pSHF mesoderm cells, wherein the pSHF mesoderm cells are MESP1+, CXCR4−, ALDH1A2+, CD1Dhigh, HOXA1+, HOXB1+, HOTAIRM1+, TBX6+, and CDX2+.

In still another aspect, pSHF mesoderm cells made by the methods described herein are provided.

In yet another aspect, methods of making posterior second heart field (pSHF) progenitor cells are provided. Such methods typically include: culturing the pSHF mesoderm cells as described herein or isolated day 4 CXCR4−ALDH+mesoderm cells in the presence of an appropriate amount of IWP2, VEGF, and retinol for about 2 to about 4 days (e.g., about 3 days), thereby producing pSHF progenitor cells, wherein the pSHF progenitor cells are ALDH1A2+, HOXB1+, HOTAIRM1+, NR2F2+, DUSP9+, and FOXF1+.

In one aspect, pSHF progenitor cells made by the methods described herein are provided.

In still another aspect, methods of making posterior second heart field (pSHF) cardiomyocytes are provided. Such methods typically include: culturing the pSHF progenitor cells described herein in the presence of an appropriate amount of retinol for about 2 to about 4 days (e.g., about 3 days) followed by culturing in base media for about 12 to about 15 days, thereby making pSHF cardiomyocytes, wherein the pSHF cardiomyocytes comprise a first population of atrial cardiomyocytes (ACMs) that are NKX2-5+, NR2F2+, and SCN5A+ and a second population of sinus venosus cardiomyocytes (SVCM) that are TBX18+ and SFRP5.

In one aspect, pSHF cardiomyocytes made by the methods described herein are provided. In some embodiments, the pSHF mesoderm, progenitor and/or cardiomyocytes can be differentiated into atrial (e.g., right and left atrial) or sinus venosus (SV) structures.

In still another aspect, methods of modelling atrial fibrillation using pSHF-derived ACMs are provided. Such methods typically include: culturing the pSHF mesoderm cells, the pSHF progenitor cells, and/or the pSHF cardiomyocytes as described herein under a variety of culture conditions and evaluating their characteristics.

In yet another aspect, methods of screening drugs are provided. Such methods typically include: contacting any of the cells described herein with a test compound; and determining the effect of the compound on the differentiation of those cells into downstream cells.

Unless otherwise defined, all technical and scientific terms used herein have the same meaning as commonly understood by one of ordinary skill in the art to which the methods and compositions of matter belong. Although methods and materials similar or equivalent to those described herein can be used in the practice or testing of the methods and compositions of matter, suitable methods and materials are described below. In addition, the materials, methods, and examples are illustrative only and not intended to be limiting. All publications, patent applications, patents, and other references mentioned herein are incorporated by reference in their entirety.

### DESCRIPTION OF DRAWINGS

FIG. 1A-1H are results from experiments showing the generation of FHF and SHF mesoderm from hPSCs. (1A) Schematic representation of the protocol used to generate cardiomyocytes from hPSCs. (1B) Representation of flow cytometric analyses of ALDH activity and CD235a/b expression in day 4 cultures generated using different concentrations of Activin A and BMP4. (1C) RT-qPCR analyses of BRY and MESP1 expression in the 3B1.5A- and 16B8A-induced cultures (n≥3). (1D and 1E) RT-qPCR analyses of the expression levels of MIXL1, FGF8, EOMES and PITX2 (1D), as well those of FOXC2, CITED1, FOXC1, FOXH1, HOXB1, HOXA1 and TBX6 in the day 3 3B1.5A- and 16B8A-induced cultures (n≥4) (1E). (1F) RT-qPCR analyses of the expression levels of TBX5, ISL1, FGF10 and TBX1 in the days 4 to 6 cultures generated with 3B1.5A and 16B8A conditions. (1G and 1H) RT-qPCR analyses of the expression levels of HAND1, TBX5, HAND2 and NR2F2 in the days 12 and 20 cultures generated with 3B1.5A and 16B8A conditions (n≥5). Statistical comparison was performed using unpaired t test; * p<0.05, ** p<0.01, *** p<0.001, **** p<0.0001. All error bars represent SEM.

FIG. 2A-2K are results from experiments showing the characterization of heterogeneity and temporal specification of hPSC-derived cardiac mesoderm populations. (2A) UMAP plots of 3B1.5A- and 16B8A-specified mesoderm populations that contain aSHF, pSHF, FHF mesoderm clusters. (2B) UMAP plots demonstrating the expression of MESP1, FOXA2, FHF mesoderm markers (EOMES and LHX1), aSHF mesoderm markers (SIX1 and FOXC2) and pSHF mesoderm markers (HOXA1 and ALDH1A2). (2C) Venn diagrams showing the proportion of species-specific and conserved gene expression patterns in hPSC-derived and mouse FHF, aSHF, and pSHF mesoderm. (2D) Dot plot showing the species-conserved markers of aSHF, pSHF and FHF mesoderm. (2E-2G) RT-qPCR analyses of the expression levels of FHF mesoderm genes TDGF1, LHX1, GSC, FGF17, BMP4, BMP2, GATA6 and GYPB (2E), pSHF mesoderm genes GAL, PCDH19, ALDH1A2, PRICKLE1, HES7, RBP1, CRABP1 and HOTAIRM1 (2F), as well as aSHF mesoderm genes PHLDA1 and CXCR4 (2G) in the day 3 3B1.5A- and 16B8A-induced cultures (n≥5). Statistical analyses were performed using unpaired t test; * p<0.05, ** p<0.01, *** p<0.001, **** p<0.0001. All error bars represent SEM. (2H) Dot plot showing the species-conserved GO terms enriched in the mouse and hPSC-derived FHF, aSHF, and pSHF mesoderm clusters. (2I) UMAP plot of integrated hPSC-derived mesoderm and human gastrulating cells colored by pre-annotated cell identity. (2J) Pseudotime ordering of hPSC-derived and human gastrulating mesoderm. (2K) Dot plot showing the expression of pSHF and aSHF mesoderm markers in the nascent mesoderm and emergent mesoderm respectively.

FIG. 3A-3G are experimental results showing the generation of distinct cardiac progenitor populations from purified mesoderm populations. (3A) Bar plots showing the top 15 genes positively and negatively correlated with GYPB, CXCR4 and ALDH1A2; Pearson correlation; p<0.05. (3B-3C) Representation of flow cytometric analyses of the expression of CXCR4, PDGFR-alpha and CD235a/b, as well as ALDH activity in the day 4 population generated with 16B8A (3B) and 3B1.5A (3C) conditions. (3D-3F) RT-qPCR analyses of the expression levels of FHF progenitor markers (3D), pSHF progenitor markers (3E) and aSHF progenitor markers (3F) in the day 5 aSHF, pSHF and FHF populations (n≥5). Statistical analyses shown in FIGS. 3D-3F were performed using one-way ANOVA with Tukey's multiple comparisons. All error bars represent SEM. * p<0.05, ** p<0.01, *** p<0.001, **** p<0.0001. (3G) Immunostaining showing the expression of HAND1 and NR2F1 in day 6 FHF, aSHF, and pSHF progenitors. (3H-3I) RT-qPCR analyses of the expression levels of RA-responsive genes (CYP26A1, HOXA1 and HOXB1) (3H), as well as the aSHF genes FGF10, FOXC1 and FOXC2 (31) in the day 5 pSHF cells treated with either DMSO or ROH. Statistical analyses were performed using unpaired t test; * p<0.05, ** p<0.01, *** p<0.001. All error bars represent SEM.

FIG. 4A-4J are experimental data showing single cell transcriptomic analyses of day 6 progenitor populations. (4A) UMAP plots of day 6 aSHF, pSHF and FHF cardiac progenitors labelled by sample name (left) and cell identity (right). (4B) Violin plots showing the expression of known aSHF (ISL1, FOXC1, FOXC2, FGF10 and WNT5A), pSHF (ISL1, HOXB1, NR2F2 and HOTAIRM1), and FHF (TBX5, HAND1, HCN4 and MYH6) progenitor markers in the aSHF, pSHF and FHF progenitor clusters; corresponding p-values can be found in Appendix B. (4C) Venn diagrams showing the proportion of species-specific and conserved gene expression patterns in hPSC-derived and mouse FHF, aSHF, and pSHF progenitors. (4D) Dot plot showing species-conserved markers of the aSHF, pSHF and FHF progenitors. (4E) Dot plot representation of species-conserved GO terms enriched in aSHF, pSHF and FHF progenitors. (4F) UMAP plots of slingshot pseudotime inference of mesoderm (day 3), late mesoderm (day 4) and progenitor (day 6) populations of aSHF and pSHF lineages. (4G) Expression of ALDH1A2, RDH10 and RARB in aSHF (purple) and pSHF (blue) lineages along pseudotime. (4H) Flow cytometric analyses of ALDH activity and CD235a/b expression in the day 5 FHF, aSHF and pSHF cultures. (4I) Quantification of the percentages of ALDH⁺ cells in the day 5 aSHF, pSHF, and FHF cultures (n=4). Analysis was performed using one-way ANOVA with Tukey's multiple comparisons. All error bars represent SEM. **** p<0.0001. (4J) RT-qPCR analyses of the expression levels of CYP26A1 and HOXB1 in the day 6 aSHF cells treated with ROH or DMSO on day 5 (n≥5). Statistical analyses were performed using unpaired t test; * p<0.05, ** p<0.01. All error bars represent SEM.

FIG. 5A-5I are experimental data showing that Bmp signaling is required for ventricular cardiomyocyte differentiation of the aSHF lineage. (5A) GSEA analysis showing the enrichment of ‘Response to BMP’ and ‘Regulation pf BMP signaling pathway’ in the hPSC-derived aSHF progenitor. (5B) Heatmap summarizing the expression of genes related to Bmp signaling in the pSHF, aSHF and FHF progenitor clusters. (5C-5E) RT-qPCR analyses of the expression levels of FGF8, ISL1, HOXB1 and HAND1 (5C), SIX1 and FOXC2 (5D), as well as UNC45B and GATA4 (5E) in the day 6 aSHF, pSHF and FHF populations treated as indicated (n≥4). (5F-5G) Flow cytometric analyses of the expression of PDGFR-beta (CD140b) and SIRP-alpha (CD172a) (5F) or MYL2 and TNNT2 (5G) in the day 20 aSHF, pSHF and FHF populations treated as indicated. (5H) Quantification of cell numbers of day 20 aSHF population treated as indicated. (5I) RT-qPCR analyses of the expression levels of IRX4, MYL2, NR2F2 and PDGFRB in the day 20 aSHF, pSHF and FHF populations treated as indicated (n≥5). Statistical analyses shown in FIG. 5 were performed using one-way ANOVA with Tukey's multiple comparisons. All error bars represent SEM. *p<0.05, ** p<0.01, *** p<0.001, **** p<0.0001.

FIG. 6A-6I are experimental data showing the characterization of transcriptional profiles of FHF-, aSHF- and pSHF-derived cardiomyocytes. (6A) UMAP plots of day 20 FHF, aSHF, and pSHF cultures that contain various cardiomyocyte subtypes labelled by sample name (left) and cell type (right). (6B) UMAP plots showing the expression of NKX2-5, TNNT2, MYL2, BMP2, HAND1, IRX1, SEMA3C, NR2F2, TBX18 and CAV1 in the day 20 hPSC-derived cardiomyocytes. (6C) Venn diagram showing the numbers of mouse and hPSC-derived RVCM and OFTCM markers; dot plot showing the top 10 species-conserved RVCM and OFTCM markers; corresponding p-values can be found in Appendix C. (6D) Dot plot showing the species-conserved GO terms enriched in the hPSC-derived and mouse RVCMs and OFTCMs. (6E) Venn diagram showing the numbers of mouse and hPSC-derived RVCM and LVCM markers; dot plot showing the top 10 species-conserved RVCM and LVCM markers; corresponding p-values can be found in Appendix C. (6F) Dot plot showing the species-conserved GO terms enriched in the hPSC-derived and mouse RVCMs and LVCMs. (6G-6H) RT-qPCR analyses of the expression levels of RVCM (6G), and LVCM genes (6H) in the day 20 aSHF-pSHF- and FHF-derived cardiomyocytes (n≥5). Statistical analyses were performed using one-way ANOVA with Tukey's multiple comparisons. All error bars represent SEM. * p<0.05, ** p<0.01, *** p<0.001, **** p<0.0001. (6I) Immunostaining of HAND1, IRX1 and TBX5 in the day 20 FHF, aSHF and pSHF cultures.

FIG. 7A-7F show experimental data of integration of hPSC-derived and human fetal cardiomyocytes and pseudotime analyses of the hPSC-derived FHF, aSHF and pSHF lineages. (7A) UMAP plots of the integrated human fetal and hPSC-derived cardiac cells labeled by data source (left), cell identity (middle) and cluster numbers (right). (7B) UMAP plots demonstrating the expression of NKX2-5, TNNT2, MYL2 and NR2F2 in the integrated data. (7C) Bar plots showing the compositions of cluster 0, cluster 1 and cluster 3 of the integrated data. (7D) Correlation matrix showing the correlation between all the human fetal and hPSC-derived cells in the clusters 0, 1 and 3; Pearson correlation; p<0.05. (7E) UMAP plot showing hPSC-derived data from various differentiation stages; UMAP plots showing pseudotime trajectories of FHF, aSHF and pSHF lineages. (7F) Schematic illustration of the development of human and mouse FHF, aSHF, and pSHF lineages. Mouse and human mesoderm populations contain molecularly and temporally distinct FHF, aSHF and pSHF sub-populations. FHF mesoderm develops FHF progenitor, which eventually generates LVCMs and AVCCMs. ASHF mesoderm develops aSHF progenitor. Following the activation of RA and BMP signaling, aSHF progenitors generate RVCMs and OFTCMs. PSHF mesoderm gives rise to ACM and SV-like cells upon the activating of RA signaling.

FIG. 8A-8F are experimental data related to FIG. 1. (8A) Flow cytometric analysis of PDGFRα expression in the day 4 cultures generated with various concentrations of BMP4 and Activin A. (8B) Violin plots showing the expression of Mesp1, early-emerging mesoderm markers (Mixll (p_adj=2.45E-48), Fgf8 (p_adj=8.47E-41), Eomes (p_adj=7.49E-12) and Pitx2 (p_adj=1.71E-43)), and late-emerging mesoderm markers (Foxc1 (p_adj=1.38E-12), Foxc2 (p_adj=8.85E-40), Foxh1 (p_adj=6.91E-17), Hoxb1 (p_adj=3.67E-6), Hoxa1 (p_adj=2.14E-5), Tbx6 (p_adj=2.45E-6), and Cited1 (p_adj=5.81E-6)) in the E6.75 and E7.25 Mesp1⁺ mouse mesoderm populations respectively; analyses were performed using Wilcoxon rank-sum test; Benjamini-Hochberg adjusted. (8C) UMAP plots of E6.75 and E7.25 mouse mesoderm populations labelled by stage and cell type. (8D) Dot plot demonstrating the markers of mouse FHF, aSHF and pSHF mesoderm; corresponding p-values were determined. (8E) Gene ontology analysis based on DEGs of mouse FHF, aSHF and pSHF mesoderm. (8F) Representation of flow cytometric analysis of CTNT expression in the day 20 3B1.5A- and 16B8A-induced cultures.

FIG. 9A-9H are experimental data related to FIG. 2. (9A) Dot plot showing the expression of markers of mesoderm, endoderm, ectoderm, paraxial mesoderm in the day 3 cells. (9B) GSEA analysis showing an enrichment of ARVC in the hPSC-derived aSHF mesoderm. (9C) UMAP plot of the integrated hPSC-derived mesoderm and human gastrulating cells labelled by data source. (9D) Correlation matrix showing the correlation between human gastrulating cells and hPSC-derived mesoderm; Pearson correlation; p<0.05. (9E) Expression of TBXT, MESP1, GATA6, BMP2, IRX1 and HOXA1 along pseudotime. (9F) UMAP plots of day 4 cells labelled by sample name (left) and cell identity (right). (9G) UMAP plots showing the expression of MESP1, FOXA2, GATA6, and PECAM1. (9H) Heatmap showing the top 10 markers of FHF, aSHF and pSHF late mesoderm, as well as those of mesoderm, endoderm and endothelium.

FIG. 10A-10I are experimental data related to FIG. 3. (10A) Violin plots showing the expression of CD1D (p_adj=8.12E-135) and ITGA3 (p_adj=3.93E-52) in the hPSC-derived FHF, aSHF and pSHF mesoderm clusters. (10B) RT-qPCR analyses of the expression levels of CD1D and ITGA3 in the day 3 3B1.5A- and 16B8A-induced cultures (n≥4); analyses were performed using unpaired t test; ** p<0.01, *** p<0.001. All error bars represent SEM. (10C-10D) Representation of flow cytometric analyses of the expression of CXCR4, ALDH, and CD1d (10C) or CD49c (10D) in the day 4 3B1.5A-induced culture. Quantification of the percentages of ALDH⁺ cells in the CD1d or CD49c high and low populations; analyses were performed using unpaired t test: ** p<0.01, *** p<0.001. All error bars represent SEM. (10E) UMAP plot showing the expression of FHF, aSHF and pSHF markers in mouse E7.75 cardiac progenitors. (10F) Immunostaining showing the expression of TBX5 and NR2F2 in day 6 FHF, aSHF, and pSHF progenitors. (10G-10H) RT-qPCR analyses of the expression levels of aSHF progenitor markers (ISL1, FGF10, SIX1 and FGF8) (10G), and pSHF progenitors (TBX5, HOXA1, HOXB1 and NR2F1) (10H) in the day 5 cells derived from CXCR4⁺CD1d^{-/low} and CXCR4⁻CD1d⁺mesoderm (n=4). Statistical analyses were performed using unpaired t test; * p<0.05, ** p<0.01, **** p<0.0001. All error bars represent SEM. (10I) Violin plots showing the expression of Aldh1a2, Rdh10, Rarb and Cyp26a1 in mouse E7.75 FHF, aSHF and pSHF progenitors.

FIG. 11A-11H are experimental data related to FIG. 4. (11A) UMAP plots showing MYH6 positive and negative cells in the hPSC-derived day 6 FHF, aSHF and pSHF cultures. (11B) Heatmap demonstrating the expression of aSHF, pSHF and FHF progenitor markers in the MYH6⁺ aSHF, pSHF and FHF cells. (11C) Violin plots showing the expression of ALDH1A2, RDH10 and RARB in hPSC-derived aSHF, pSHF and FHF progenitor clusters. (11D) UMAP plots showing ALDH1A2 positive and negative cells in the hPSC-derived day 6 aSHF and pSHF cultures. (11E) Dot plot showing the differential expression of aSHF (purple) and pSHF (blue) genes in ALDH1A2-expressing pSHF and aSHF cells. (11F) Dot plot showing the GOs enriched in the ALDH1A2-expressing pSHF and aSHF progenitors respectively. (11G) UMAP plots of the re-clustered aSHF progenitor cells that show the expression of myocyte markers (NKX2-5, ISL1, GATA6 and TNNT2) and aSHF progenitor markers (FGF8, FOXC2, ALDH1A2 and TBX1). (11H) UMAP plot showing the expression of Isl1, Nkx2-5, Gata6, Tnnt2, Fgf8, Foxc2, Aldh1a2 and Thx/in the E7.75 mouse aSHF sub-clusters.

FIG. 12A-12I are experimental data related to FIG. 6. (12A) UMAP plots showing the presence of various myocyte subtypes in the E9.25 mouse heart. (12B) Venn diagram showing the numbers of mouse and hPSC-derived ACM and SVCM markers; dot plot showing the top 10 species-conserved ACM and SVCM markers; corresponding p-values can be found in Appendix C. (12C) Dot plot showing the conserved GO terms enriched in the hPSC-derived and mouse ACMs and SVCMs. (12D) RT-qPCR analyses of the expression levels of TBX18, HOTAIRM1 and NR2F2 in the day 20 aSHF, pSHF and FHF cultures (n≥5). Statistical analyses were performed using one-way ANOVA with Tukey's multiple comparisons. All error bars represent SEM. *** p<0.001, **** p<0.0001. (12E) Venn diagram showing the numbers of mouse and hPSC-derived AVCCM and LVCM markers; dot plot showing the top 10 species-conserved AVCCM and LVCM markers; corresponding p-values can be found in Appendix C. (12F) Dot plot showing the conserved GO terms enriched in the hPSC-derived and mouse AVCCMs and LVCMs. (12G) GSEA analysis showing the enrichment of ARVC genes in the hPSC-derived RVCMs. (12H) Immunostaining of HEY2 and GJA1 in the day 20 FHF, aSHF and pSHF cardiomyocytes. (12I) Quantification of GJA1 and IRX1 expression in the day 20 FHF, aSHF and pSHF cultures. Statistical analyses were performed using one-way ANOVA with Tukey's multiple comparisons. All error bars represent SEM. *** p<0.001.

FIG. 13A-13G are experimental data related to pseudotime analyses of hPSC-derived FHF, aSHF and pSHF lineages. (13A) UMAP plot showing hPSC-derived cells of various lineages and stages. (13B) UMAP plot of pseudotime trajectory of FHF lineage inferred by Monocle. (13C) Heatmap showing gene modules enriched in various FHF populations. (13D) UMAP plot of pseudotime trajectory of aSHF lineage inferred by Monocle. (13E) Heatmap showing gene modules enriched in various aSHF populations. (13F) UMAP plot of pseudotime trajectory of pSHF lineage inferred by Monocle. (13G) Heatmap showing gene modules enriched in various pSHF populations.

FIG. 14A-14E are experimental data related to modeling FHF, aSHF and pSHF lineage development with HES3 line. (14A) RT-qPCR analyses of the expression levels of FHF, aSHF and pSHF mesoderm markers in the day 3 HES3-derived FHF (15B8A) and SHF (3B1A) cultures (n≥5). (14B-14C) Representation of flow cytometric analyses of the expression of CXCR4, PDGFRα and CD235a/b, as well as the ALDH activity in the day 4 cultures generated with 15B8A (14B) and 3B1A (14C) conditions. (14D) RT-qPCR analyses of the expression levels of FHF, aSHF and pSHF progenitor markers in the day 5 cultures derived from aSHF, pSHF and FHF mesoderm (n≥5). (14E) RT-qPCR analyses of the expression levels of LVCM, RVCM and pSHF-derived myocyte markers in the day 20 aSHF, pSHF and FHF cultures (n≥5). Statistical analyses were performed using one-way ANOVA with Tukey's multiple comparisons. All error bars represent SEM. * p<0.05, ** p<0.01, *** p<0.001, **** p<0.0001.

### DETAILED DESCRIPTION

The cardiomyocyte subtypes in the different chambers derive from distinct progenitors known as the first heart field (FHF) and second heart field (SHF) progenitors. These progenitors are specified by E7.5-E8.0 and are distinguished at this stage by gene expression patterns and their position within cardiac crest region of the developing embryo. The FHF progenitors, identified by the expression of Hcn4, Hand1 and Tbx5, give rise predominantly to left ventricular cardiomyocytes (LVCMs), atrioventricular canal cardiomyocytes (AVCCMs) along with some atrial cardiomyocytes (ACMs). SHF progenitors, distinguished by expression of Isl1, Fgf10 and Fgf8, generate the majority of the right ventricular (RVCMs) and ACMs as well as the outflow tract (OFT) and inflow tract (IFT) cells collectively referred to as sinus venosus (SV) structures. Further delineation of anterior-posterior patterning within the SHF population revealed a degree of heterogeneity indicative of distinct progenitors with different fates. Those positioned anteriorly, the anterior SHF progenitors (aSHF) are characterized by expression of Tbx1, Fgf8 and Fgf10 and contribute to RVCMs and OFT lineages. By contrast, the progenitors found in the posterior region (pSHF) are identified by expression of Hoxb1 and Nr2f2 and give rise to ACMs and SV structures.

Studies aimed at identifying the signaling pathways that control FHF and SHF development have largely focused on the mesoderm and progenitor stages of development and have provided evidence that the two populations are regulated differently. BMP signaling plays a pivotal role in initiating the cardiac program of FHF lineage, which differentiates rapidly and forms the first contracting population within the heart tube. The SHF progenitors, by contrast, are exposed to an FGF/Wnt environment that promotes their proliferation, thereby delaying their differentiation. Following this proliferative phase, these cells differentiate and give rise to derivative cell types including ACMs, SV/IFT cells, OFTCMs and RVCMs. Specification of the atrial lineage from the pSHF progenitors is dependent on RA signaling whereas the generation of RVCMs from the aSHF progenitors is regulated by BMP signaling. Regulation of FHF/SHF mesoderm induction is less well understood, but likely will involve the pathways that control gastrulation, including BMP, Nodal, Wnt and FGF.

To map human cardiovascular development from the perspective of FHF and SHFs, comprehensive single-cell RNA sequencing (scRNA-seq) analyses were carried out on mesoderm, progenitor and contracting cardiomyocyte populations induced under different conditions. The findings from these analyses enabled the identification and characterization of distinct FHF, aSHF, and pSHF lineages, beginning at the stage of mesoderm induction and progressing through progenitors to the respective derivative cardiomyocyte subtypes. Thus, this disclosure describes an hPSC-based platform for multi-lineage human cardiogenesis from mesoderm specification to terminal myocyte differentiation. Such a platform can be used to produce different types of human cardiomyocytes.

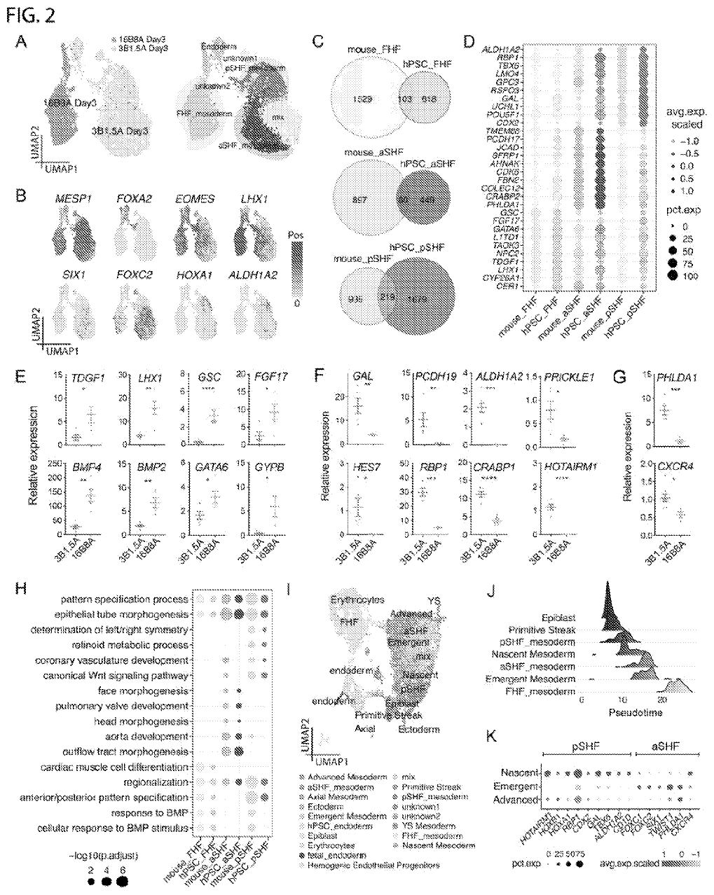

It is shown herein that different levels of Activin/Nodal and BMP signaling play a pivotal role in the generation of the FHF and SHF populations from hPSCs. In addition to the BMP and Activin/Nodal pathways, it is shown herein that human pSHF mesoderm express components of the canonical Wnt pathway (FIG. 2H). In the hPSC differentiation protocol described herein, it is possible that endogenous levels of Wnt signaling in the presence of added BMP and Activin A agonists are sufficient to induce the pSHF lineage. Although we were able to establish different signaling environments in vitro with different concentrations of pathway agonists, the temporal patterns of FHF and SHF mesoderm are difficult to recapitulate in vitro. However, comparison of the hPSC-derived populations to those found in the human gastrulating embryo indicate that they likely represent temporally distinct subsets of mesoderm, with the pSHF mesoderm showing transcriptomic similarity to nascent mesoderm and the aSHF mesoderm to emergent mesoderm. Collectively, these observations indicate that the development of the hPSC-derived cardiac mesoderm subtypes are induced, in part, through different levels of TGF-beta signalling.

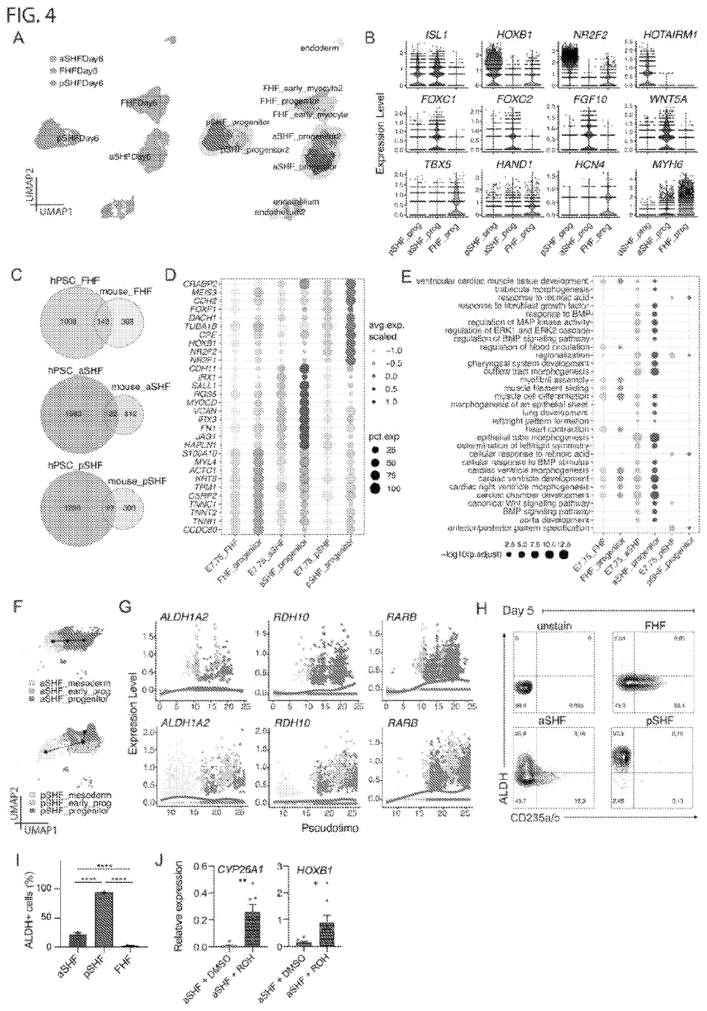

Although lineage tracing and retrospective studies established the lineage relationship between distinct mesoderm and cardiomyocyte subtypes, the transition from mesoderm to cardiovascular progenitors remains largely uncharacterized. Through the ability to isolate the hPSC-derived mesoderm subpopulations using markers identified from the scRNA-seq analyses described herein, we were able to formally establish lineage-specific mesoderm-progenitor relationships. It was found that CD235a/b⁺ mesoderm gives rise to a population that that displays a molecular profile of FHF progenitors, CXCR4⁺ALDH⁻ mesoderm to aSHF progenitors and CXCR4⁻ALDH⁺ (or CXCR4⁻CD1D⁺) mesoderm to pSHF progenitors. The analyses of the progenitor populations described herein identified several new insights into the regulation of derivative cell populations. The first is that the aSHF lineage upregulates ALDH activity (ALDH1A2) at the progenitor stage, approximately 24 hours following its upregulation in the pSHF lineage. These findings strongly suggest that progenitors of both the pSHF and aSHF lineages are ALDHA2⁺, distinguishing them from the FHF progenitors that are ALDH1A2⁻ (FIG. 4H). The second finding is that the aSHF population expresses components of the BMP pathway. Based on this observation, we were able to demonstrate that signaling through this pathway is required for the generation of VCMs from these progenitors.

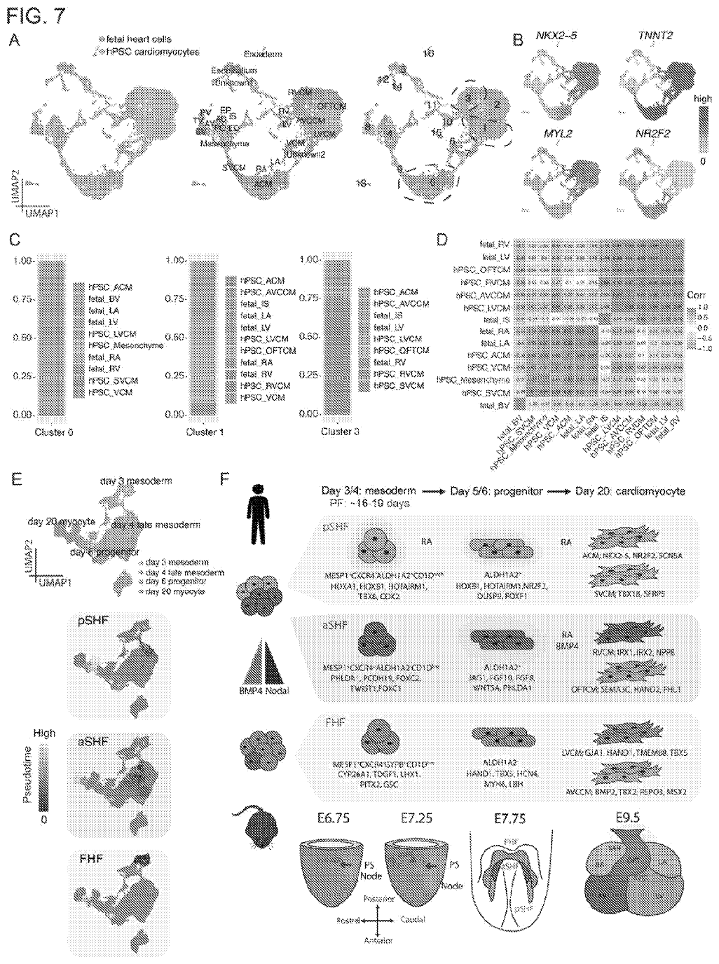

In addition to establishing mesoderm-progenitor relationships, access to isolated populations of mesoderm have enabled us to track the origin of the different human cardiomyocyte subtypes and to establish a developmental map of human heart field lineages (FIG. 7F). These findings show that the human lineages display developmental potential, with the FHF giving rise to LVCMs and AVCCMs, the aSHF giving rise to RVCMs and OFTCMs, and the pSHF giving rise to ACMs and SVCMs (FIG. 7E). Significantly, methods for generating RVCMs, LVCMs, OFTCMs and AVCCMs previously have not been reported.

Distinguishing cardiomyocyte subtypes, in particular those that form left versus right chambers such as LVCMs and RVCMs in the absence of chamber structures can be challenging, as these cells express many ventricular lineage genes in common. Three lines of evidence from the findings herein, however, indicate that we have generated LVCMs and RVCMs. The first is the demonstration that these VCMs develop from different subpopulations of mesoderm; the LVCMs from the FHF mesoderm and the RVCMs from the aSHF mesoderm. The second is through species-conserved, chamber-specific gene expression patterns that distinguish the putative LVCMs and RVCMs. Third, the demonstration that the aSHF mesoderm as well as the RVCMs expressed genes associated with ARVC, a disease that primarily targets the right ventricle.

The ability to generate distinct populations of LVCMs and RVCMs from hPSCs is important for both cell therapy and disease modelling applications. For example, LVCMs are likely the best cell type for transplantation to remuscularize an infarcted region of the left ventricle, whereas RVCMs would be the appropriate population for modeling ARVC. The identification of subpopulations that show gene expression profiles of OFTCMs and AVCCMs is a first step to establishing optimized protocols for the generation of these cardiomyocyte subtypes. Access to these cells will provide a platform for modeling diseases that target these regions of the heart, including atrioventricular canal defect, outflow tract ventricular arrhythmias and persistent truncus arteriosus.

The map of human cardiac lineage development described herein differs from that of the mouse in that ACMs were not detected in the human FHF-derived population, whereas lineage tracing studies in the mouse suggest that the FHF does contribute to atrial formation.

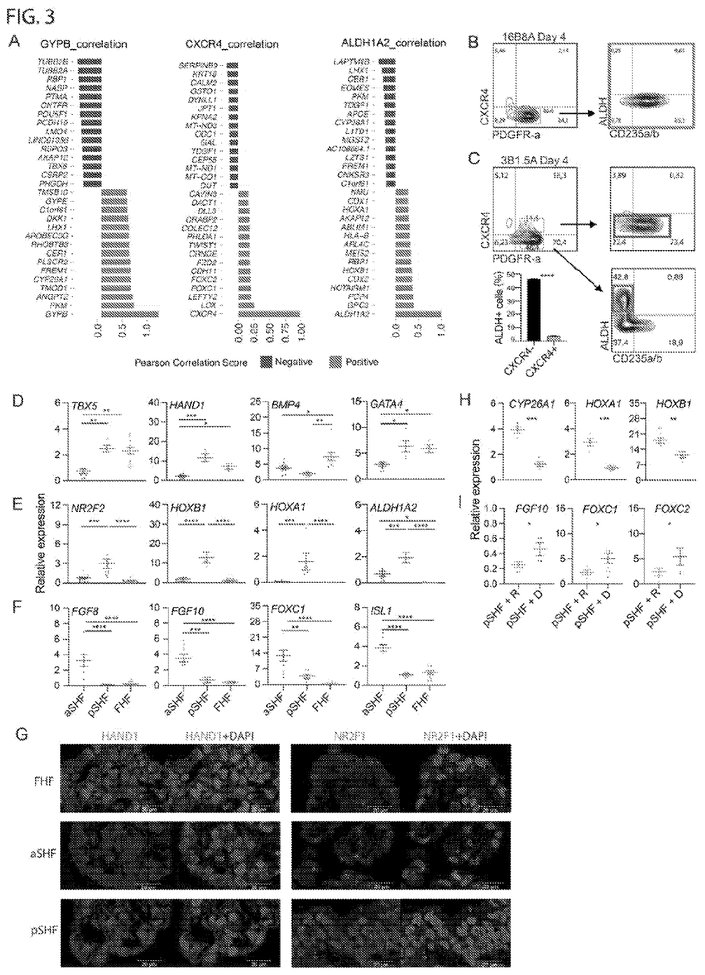

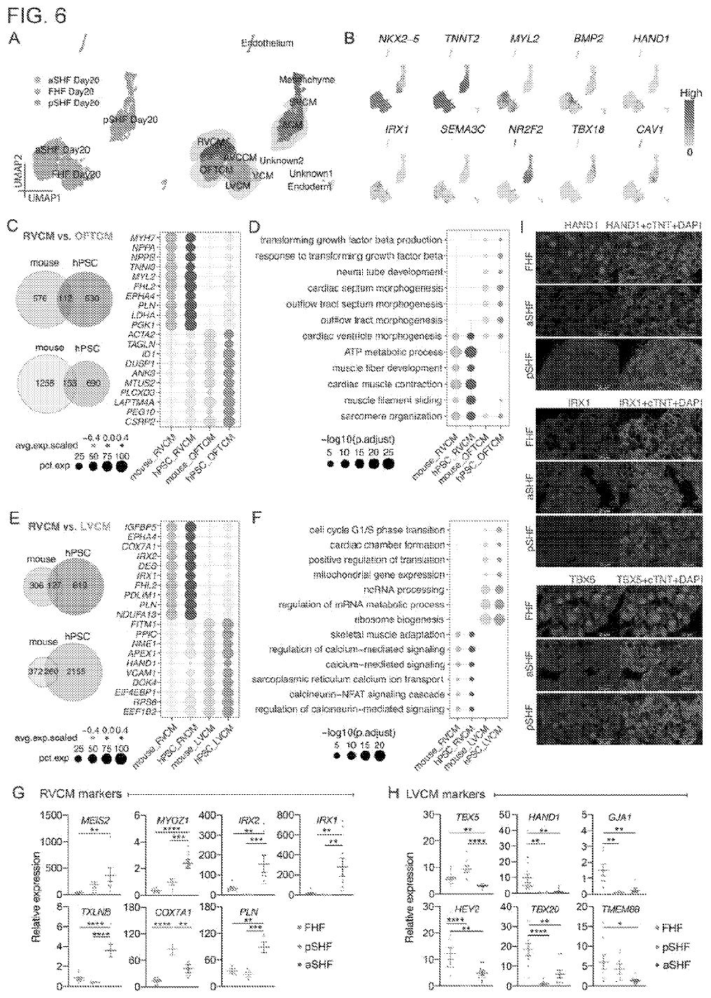

These differences could reflect differences between mouse and human or result from suboptimal conditions for atrial development the FHF cultures. Alternatively, they may be due to the different approaches used to establish the potential of the population. In vivo lineage tracing studies or in vitro studies using reporter hPSC lines define cardiac lineage potential based on the expression of genes such as ISL1 and TBX5, which do show SHF and FHF bias expression patterns, respectively. The findings described herein, however, indicate that these patterns in the hPSC-derived populations are stage specific, as it was found that restriction of ISL1 expression to the SHF occurs beyond the progenitor stage of differentiation (day 5); prior to this, its expression was observed in both the FHF and SHF in day 4 late mesoderm populations. Similarly, it was found that the FHF marker TBX5 is expressed in the pSHF progenitors and derivative cardiomyocytes (FIGS. 3D and 6H). Given these patterns, lineage tracing studies based on TBX5 expression or analyses of NKX2-5⁺TBX5⁺ hPSC-derived populations are likely to assign atrial cells to the FHF lineage. In this study, the human FHF, aSHF and pSHF lineages were defined based on the developmental potential of isolated mesoderm populations and expression patterns of several sets of genes along differentiation, an approach that is not dependent on specific gene expression patterns.

Through the use of precise stage-specific induction strategies and extensive transcriptomic analyses, human FHF, aSHF and pSHF cardiac lineages were identified and characterized that, together, establish a comprehensive map of human cardiovascular development. This map identifies stage-specific molecular signatures for each of the lineages, enabling the characterization and identification of populations generated from different hPSC lines using different induction protocols (Table 1). Access to these different cell types will provide unprecedented opportunities for detailed genetic and epigenetic studies on human cardiac development, for modeling cardiovascular diseases that target specific regions of the heart, and for developing cell-based therapies with appropriate chamber specific populations.

TABLE 1
Summary of Stage and Lineage Markers

| | | |
|---|---|---|
| Mesoderm | | |
| | FHF | GYPB, EOMES, CYP26A1, LHX1, TDGF1, LITD1, BMP2, MIXL1, |
| | | GSC, FGF17 |
| | aSHF | CXCR4, FOXC2, FOXC1, PHLDA1, IRX1, SIX1, ITGA3, IRX3 |
| | pSHF | ALDH1A2, HOTAIRM1, HES7, CDX2, GAL, TBX6, HOXB1, CD1D, |
| | | RDH10 |
| Progenitor | | |
| | FHF | TBX5, TBX20, HAND1, HCN4, LBH, MIF1, TNNT2, MYH6 |
| | aSHF | FOXC2, FOXC1, PHLDA1, IRX1, SIX1, ITGA3, IRX3 |
| | pSHF | NR2F2, NR2F1, TBX18, HOXB1, HOXA1, DUSP9, DACH1, VEGFC, |
| | | FOXF1 |
| Cardiomyocyte | | |
| | LVCM (FHF) | HAND1, GJA1, TBX5, TBX20, TMEM88, VCAM1, EFNB3 |
| | AVCCM (FHF) | BMP2, TMSB4X, TBX3, RSPO3, CPNE5, MSX2, FAM78A |
| | RVCM (aSHF) | PLN, FHL2, IRX1, IRX2, NPPB, NPPA, TXLNM, MYOZ2 |
| | OFTCM (aSHF) | HAND2, MEF2C, VCAN, SEMA3C, CFC1, WNT5A, FHL1, CDH11 |
| | ACM (pSHF) | NPPA, MYL4, KCNJ5, CRYAB, NKX2-5, CAV1 |
| | SVCM (pSHF) | CRABP2, SFRP5, SFRP1, TBX18, RBP1, FOXP2, COL1A2, PLAT |

The term “pluripotent stem cell” as used herein refers to a cell with the capacity to differentiate into cells of the three germ cell layers. Pluripotency is also evidenced by the expression of embryonic stem (ES) cell markers (e.g., POU5F1+, SOX2+, NANOG+, SSEA3+. SSEA4+, and SSEA5+). Suitable pluripotent cells for use herein include embryonic stem cells (ESCs; e.g., human ESCs) such as, for example, mesoderm cells (e.g., human mesoderm cells that express, for example, KDR+CD56+CD34−), induced pluripotent stem (iPS) cells (e g., human iPS cells), or cells from embryoid bodies (e.g., cells from human embryoid bodies).

In accordance with the present invention, there may be employed conventional molecular biology, microbiology, biochemical, and recombinant DNA techniques within the skill of the art. Such techniques are explained fully in the literature. The invention will be further described in the following examples, which do not limit the scope of the methods and compositions of matter described in the claims.

### EXAMPLES

### Example 1—Directed Differentiation of hPSCs

For differentiating cardiomyocytes from hPSCs, published embryoid body (EB)-based protocol was used (Lee et al., 2017, Cell Stem Cell, 21:179-94). Briefly, at day 0 of the protocol, hPSCs at 80% confluency were dissociated into single cells (TrypLE, ThermoFisher) and re-aggregated to generate EBs in the base media (StemPro-34 media (ThermoFisher) containing 1% penicillin/streptomycin (ThermoFisher), 2 mM L-glutamine (ThermoFisher), 150 μg/mL transferrin (ROCHE), 50 μg/mL ascorbic acid (Sigma), and 50 μg/mL monothioglycerol (Sigma)) supplemented with 10 μM ROCK inhibitor Y-27632 (TOCRIS) and 1 ng/ml rhBMP4 (R&D) for 20 hours on an orbital shaker (70 rpm). Cultures were incubated in a low oxygen environment (5% CO₂, 5% O₂, 90% N₂).

At day 1, the EBs were transferred to mesoderm induction media consisting of base media, 5 ng/mL rhbFGF (R&D), and various concentration of rhBMP4 (R&D) and rhActivinA (R&D) as described in the results section. At day 3, the EBs were transferred to the media consisting of base media, 2 μM Wnt inhibitor IWP2 (TOCRIS) and 10 ng/ml rhVEGF (R&D). From day 5 to day 12, the EBs were transferred to base media with 5 ng/mL rhVEGF. From day 12 to day 20, the EBs were transferred to base media and cultured in a normoxic environment (5% CO₂, 20% O₂). For pSHF lineage differentiation, the day 4 sorted pSHF cells were treated with 2 μM Retinol (Sigma) from day 4 to day 8. Similarly, the sorted aSHF cells were treated with 2 μM Retinol and 10 ng/mL rhBMP4 from day 5 to day 8 of differentiation.

### Example 2—Flow Cytometry

Cells of early stages (day 3 to day 6) were dissociated with TrypLE for 3-5 minutes at room temperature to single cells, which were then filtered and transferred to IMDM media. Day 20 EBs were dissociated with 0.5 mg/ml collagenase type 2 (Worthington) in HANKs buffer for 1.5 hours at 37° C. The filtered day 20 cells were then transferred to FACS buffer consisting of PBS with 5% fetal calf serum (Wisent) and 0.02% sodium azide. The following antibodies were used for staining samples obtained from various stages of differentiation: anti-PDGFRa-PE (R&D Systems, 1:20), anti-CD235a/b-APC (BD PharMingen, 1:200), anti-SIRPa-PeCy7 (Biolegend, 1:2000), anti-CXCR4 (Biolegend, 1:100), anti-CDId-PE (Biolegend, 1:100), anti-CD49c-PE (ThermoFisher, 1:100), anti-cardiac isoform of CTNT (ThermoFisher Scientific, 1:2000), or anti-myosin light chain 2 (Abcam, 1:1000). For unconjugated primary antibodies, the following secondary antibodies were used for detection: goat anti-mouse IgG-APC (BD Pharmigen, 1:250), or donkey anti-rabbit IgG-PE (Jackson ImmunoResearch, 1:250). Detailed antibody information is described in Table 2.

TABLE 2
Resources

| | | |
|---|---|---|
| REAGENT or RESOURCE | SOURCE | IDENTIFIER |

Antibodies

| | | |
|---|---|---|
| Mouse monoclonal to SIRP-alpha | Biolegend | Cat.# 323808; RRID: AB_1236443 |
| (clone SE5A5), PcCy7 conjugated | | |
| Mouse monoclonal to CD140b | BD Horizon | Cat.# 564124; RRID: AB_2738609 |
| (PDGFRbeta) (clone 28D4), BV421 | | |
| conjugated | | |
| Mouse monoclonal to PDGFRalpha, | BD Pharmingen | Cat.# 556002; RRID: AB_396286 |
| PE conjugated | | |
| Mouse monoclonal to CD235a/b | BD PharMingen | Cat.# 551336; RRID: AB_398499 |
| (GYPB) (clone HIR2), APC | | |
| conjugated | | |
| Mouse monoclonal to human CD49C | ThermoFisher | Cat.# 17-0494-42; RRID: AB_10670221 |
| (ITGA3), APC conjugated | | |
| Mouse monoclonal to human CDID, | BioLegend | Cat.# 350306; RRID: AB_10641845 |
| PE conjugated | | |
| Mouse monoclonal to CTNT (clone | ThermoFisher | Cat.# MS-295-P; RRID: AB_11000742 |
| 13-11), non-conjugated | | |
| Rabbit polyclonal to MLC2V (clone | Abcam | Cat.# ab79935; RRID: AB_1952220 |
| 13-11), non-conjugated | | |
| Monoclonal mouse ant-Human | BioLegend | Cat.# 306518; RRID: AB_11146018 |
| CD184 (CXCR4) Antibody | | |
| Polyclonal Rabbit anti-Human | LSBio | Cat. # LS-C118745 |
| HAND1 Antibody | | |
| Polyclonal Rabbit anti-TBX5 | ThermoFisher | Cat. # 42-6500; RRID: AB_2533533 |
| Antibody | | |
| Monoclonal Mouse anti-Human | R&D systems; Bio-Techne | Cat. # PP-H7147-00 |
| NR2F2 Antibody | | |
| Monoclonal Mouse anti-Human | R&D systems; Bio-Techne | Cat. # PP-H8132-00 |
| NR2F1 Antibody | | |
| Polyclonal Rabbit anti-Human IRX1 | ThermoFisher | Cat. # PA5-60261; RRID: AB_2642783 |
| Antibody | | |
| Polyclonal Rabbit anti-Human HEY2 | Proteintech | Cat. # 10597-1-AP |
| Antibody | | |
| Polyclonal Rabbit anti-Human CX43 | Abcam | Cat. #AB11370 |
| (GJA1) Antibody | | |
| Goat anti-mouse IgG (H + L), APC | BD Pharmingen | Cat.# 550826; RRID: AB_398465 |
| conjugated | | |
| Donkey anti-rabbit IgG (H + L), PE | Jackson ImmunoResearch | Cat.# 711-116-152; RRID: AB_2340599 |
| conjugated | | |
| Donkey anti-rabbit IgG (H + L), | ThermoFisher | Cat.# A21206; RRID: AB_2535792 |
| AlexaFluor488 | | |
| Donkey anti-mouse IgG (H + L), | ThermoFisher | Cat.# A21202; RRID: AB_141607 |
| AlexaFluor488 | | |
| Donkey anti-rabbit IgG (H + L), | ThermoFisher | Cat.# A31572; RRID: AB_162543 |
| AlexaFluor555 | | |
| Donkey anti-mouse IgG (H + L), | ThermoFisher | Cat.# A31570; RRID: AB_2536180 |
| AlexaFluor555 | | |

Chemicals, Peptides, and Recombinant Proteins

| | | |
|---|---|---|
| Penicillin/streptomycin | ThermoFisher | Cat.# 15070063 |
| L-glutamine | ThermoFisher | Cat.# 25030081 |
| Non-essential amino acids | ThermoFisher | Cat.# 11140-050 |
| Transferrin | ROCHE | Cat.# 10652202001 |
| Ascorbic acid | Sigma | Cat.# A4544-100G |
| Monothioglycerol | Sigma | Cat.# M-6145-25ml |
| beta-Mercaptoethanol | ThermoFisher | Cat.# 21985-023 |
| ROCK inhibitor Y-27632 | Tocris | Cat.# 1254 |
| Recombinant human BMP4 | R&D | Cat.# 314-BP |
| Recombinant human ActivinA | R&D | Cat.# 338-AC |
| Recombinant human bFGF | R&D | Cat.# 233-FB |
| Recombinant human VEGF | R&D | Cat.# 293-VE |
| Retinol | Sigma | Cat.# R7632 |
| IWP2 (Wnt inhibitor) | Tocris | Cat.# 3533 |
| LDN 193189 (BMP inhibitor) | Tocris | Cat.# 6053 |
| Collagenase type2 | Worthington | Cat.# LS004-176 |
| DAPI | Biotium | Cat.# 40043 |
| Fetal calf serum (FCS) | Wisent | Cat.# 080-150 |
| Bovine serum albumin (BSA) | Sigma | Cat.# A1470 |

Critical Commercial Assays

| | | |
|---|---|---|
| RNAqueous-micro kit with RNase- | Ambion | Cat.# AM1931 |
| free Dnase treatment | | |
| Superscript III Reverse Transcriptase | ThermoFisher | Cat.# 18080044 |
| kit | | |
| QuantiFast SYBR Green PCR kit | QIAGEN | Cat.# 204057 |
| Aldefluor assay kit | STEMCELL Technologies | Cat.# 1700 |
| 10x Genomics Single Cell 3′ v3.1 | 10x Genomics | Single Cell 3′ v3.1 |
| Reagent Kit | | |

Deposited Data

| | | |
|---|---|---|
| mouse mesoderm scRNA-seq | (Lescroart F et al., 2018) | GSE100471 |
| human gastrulation scRNA-seq | (Richard C. V. Tyser et al., | http://www.human-gastrula.net/ |
| | 2020) | |
| mouse E7.75 - E9.25 cardiac cell | (T. Yvanka de Soysa et al., | GSE126128 |
| scRNA-seq | 2019) | |
| human fetal heart scRNA-seq | (Cui et al., 2019) | GSE106118 |
| hPSC-derived cardiac mesoderm, | This study | |
| early progenitor, progenitor and | | |
| cardiomyocyte scRNA-seq | | |

Experimental models: Cell Lines

| | | |
|---|---|---|
| Human ESC: HES3-NKX2-5^{eGFP/w} | Gift from Drs. E. Stanley | NA |
| line | and A. Elefanty, Monash | |
| | University, AU (Elliott et | |
| | al., 2011) | |
| Human ESC: HES2 line | WiCell | Cat.# ES02 |

Oligonucleotides

| | | |
|---|---|---|
| PCR primer sequences | This study | Table 3 |

Software and Algorithms

| | | |
|---|---|---|
| FlowJo | Tree Star | https://www.flowjo.com |
| FV10-ASW | Olympus | https://www.olympus-lifescience.com |
| GraphPad Prism | GraphPad Software | http://www.graphpad.com/scientific- |
| | | software/prism |
| 10x Chromium Single Cell Software | | https://support.10xgenomics.com/single-cell- |
| Suite v3.1 | | gene-expression/software/pipelines/ |
| | | latest/what-is-cell-ranger |
| R CRAN, Version 3.6.2 | | https://cran.r-project.org/ |
| Batchelor | (Haghverdi L et al., 2018) | |
| Seurat v3.2.2 | (Tim Stuart et al., 2019) | https://github.com/satijalab/seurat |
| Monocle3 | (Cao et al., 2019; Trapnell et | http://cole-trapnell-lab.github.io/monocle- |
| | al., 2014) | release/ |
| ClusterProfiler v3.18 | (Yu et al., 2012) | https://github.com/YuLab-SMU/clusterProfiler |
| MsigDB v7.2.1 | (Aravind Subramanian et al., | |
| | 2005; Arthur Liberzon et al., | |
| | 2015) | |
| Enrichplot v1.10.2 | (Yu G et al., 2021) | https://yulab-smu.top/biomedical-knowledge- |
| | | mining-book/ |
| Slingshot | (Street K et al., 2018) | https://github.com/kstreet 13/slingshot |
| BiomaRt | (Durinck S et al., 2009) | https://m.ensembl.org/info/data/biomart/ |
| | | biomart_r_package.html |
| Org.Mm.eg.db | (Carlson M, 2019) | |
| Org.Hs.eg.db | (Carlson M, 2019) | |
| SingleCellExperiment | (Amezquita R et al., 2020) | https://github.com/drisso/SingleCellExperiment |
| Ggplot2 | (Wickham H et al., 2016) | https://ggplot2.tidyverse.org/ |
| dplyr | (Hadley Wickham et al., | https://dplyr.tidyverse.org/ |
| | 2018) | |
| Ggcorrplot | (Alboukadel Kassambara, | |
| | 2016) | |

Other

| | | |
|---|---|---|
| StemPro-34 media (kit) | ThermoFisher | Cat.# 10639011 |
| DMEM/F12 | Cellgro | Cat.# 10-092-CV |
| Hanks Buffered Salt solution | GIBCO | Cat.# 14175-079 |
| KnockOut scrum replacement | ThermoFisher | Cat.# 10828028 |
| TrypLE | ThermoFisher | Cat.# 12605010 |
| 96-well clear flat bottom TC-treated | Falcon | Cat.# 353072 |
| culture microplate | | |
| 24-well clear flat bottom TC-treated | Falcon | Cat.# 353047 |
| culture microplate | | |
| 96-well clear flat bottom ultra-low | Corning | Cat.# 3474 |
| attachment microplate | | |
| Petri dishes 60 × 15 mm | VWR | Cat.# 25384-090 |
| Micro cover glasses 12 mm | VWR | Cat.# 89015-725 |
| ProLong diamond antifade mountant | ThermoFisher | Cat.# P36965 |

To stain live cells with cell-surface protein, dissociated single cells were stained for 15 minutes at room temperature in FACS buffer and washed twice before they were subject to further analyses. For intracellular staining, cells were fixed for 15 min at 4° C. in PBS containing 4% PFA followed by permeabilization using 90% methanol for 15 min at 4° C. The permeabilized cells were washed with PBS containing 0.5% BSA (Sigma) twice and stained with unconjugated primary antibodies in FACS buffer for 18 hours at 4° C. Stained cells were washed with PBS with 0.5% BSA and stained with proper secondary antibodies in FACS buffer for 30 mins at 4° C. Following washing steps, stained cells were processed with the Fortessa (BD) and analyzed using FlowJo software (Tree Star). For cell sorting, stained cells were kept in StemPro-34 media and sorted using Influx (BD), FACSAriall (BD), MoFlo-XDP (BD) and FACSAria Fusion (BD). Data were analyzed using FlowJo software (Tree Star).

### Example 3—Aldefluor Assay

The ALDEFLUOR™ assay (STEMCELL Technologies) was used to detect the cellular ALDH function as an indicator of cell autonomous production of retinoic acid. To perform this assay, cells were dissociated using the method described in ‘Flow Cytometry’ and then stained in the aldefluor assay buffer containing 0.1% BSA and BAAA substrate (0.12 mg/ml) for 40 min at 37° C. This step was done in an environment free of light. Aldehyde dehydrogenase inhibitor DEAB (0.75 nM) was added in the negative control. To end the reaction, cells were washed with cold wash media consisting of IMDM and 10% aldefluor assay buffer. During analyses, the cells were kept in cold wash media. For SHF mesoderm sorting, which requires the ALDEFLUOR™ assay, stained cells were maintained in cold StemPro-34 containing 10% aldefluor assay buffer throughout the sorting process. The sorted cells were collected and re-aggregated in base media containing 1 mM IWP2 and 5 ng/mL rhVEGF.

### Example 4—Quantitative Real-Time PCR

RNA extraction was performed using the RNAqueous-micro Kit (Invitrogen). Purified RNA was treated with RNase-free DNase (Invitrogen), and then reverse transcribed into cDNA using oligo (dT) primers and random hexamers and iscript Reverse Transcriptase (ThermoFisher). QRT-PCR was performed on an EP Real-Plex MasterCycler (Eppendorf) using QuantiFast SYBR Green PCR kit (QIAGEN). The copy number of each gene was determined based on a standard curve plotted with human genome DNA with known copy number. The relative expression levels of genes were obtained by normalizing their copy number to that of TBP. The detailed primer sequences are provided in Table 3.

TABLE 3
RT-qPCR primer sequences

| | | SEQ | | SEQ |
|---|---|---|---|---|
| | | ID | | ID |
| Gene | Forward 5′-3′ | NO | Reverse 5′-3′ | NO |
| BRY (T) | TGTCCCAGGTGGCTTACAGATGAA | 1 | GGTGTGCCAAAGTTGCCAATACAC | 2 |
| MESP1 | AGCCCAAGTGACAAGGGACAACT | 3 | AAGGAACCACTTCGAAGGTGCTGA | 4 |
| MIXL1 | CTGTGCTCCTGGAACTGAAA | 5 | TGGGAGCTAGAGTCAGAGATG | 6 |
| FGF8 | CTGCGTCTTCACGGAGATT | 7 | GGTGAAGGCCATGTACCAG | 8 |
| EOMES | CCCACTACAATGTGTTCGTAGA | 9 | TGCATGTTATTGTCGGCTTTG | 10 |
| LHX1 | GTTTGTCTCCGGATTCCCAA | 11 | CAGGTTCTGGTCGTCATTCTC | 12 |
| FOXC2 | CAACATGTTCGAGAACGGCAGCTT | 13 | CGCTCTTGATCACCACCTTCTTCT | 14 |
| GATA4 | CGAATGACGGCATCTGTTTGCCAT | 15 | ATTTGGTATTAGGGATGCAGGGCG | 16 |
| CITED1 | TCTCCAATAGGCTCTCCTACAA | 17 | TAAGTTTCTGCAGGTGCATACT | 18 |
| FOXC1 | ACCCGGACTCCTACAACAT | 19 | TCCTTCTCCTCCTTGTCCTT | 20 |
| HEY2 | GAGTGAGAGAGTCGTGTTTC | 21 | ACTTCTGTCCCTTTCCTTTC | 22 |
| FOXH1 | CCACCTCCTACTTGCCTATCTA | 23 | GCTGGTTGACGGACACTG | 24 |
| ISL1 | GAAGGTGGAGCTGCATTGGTTTGA | 25 | TAAACCAGCTACAGGACAGGCCAA | 26 |
| JAG1 | TGCTACAACCGTGCCAGTGACTAT | 27 | AGTGGTCTTTCAGGTGTGAGCAGT | 28 |
| HOXB1 | AGAAGGAGACGGAGGCTATT | 29 | GGTCTGCTCGTTCCCATAAG | 30 |
| MYL2 | TGTCCCTACCTTGTCTGTTAGCCA | 31 | ATTGGAACATGGCCTCTGGATGGA | 32 |
| HOXA1 | GAAAGTTGGAGAGTACGGCTAC | 33 | TGTTGAAGTGGAACTCCTTCTC | 34 |
| TBX6 | GCCCGCTACTTGTTTCTTCT | 35 | GTGAATGTAGACACGGTCAGG | 36 |
| TBX5 | ACAAAGTGAAGGTGACGGGCCTTA | 37 | ATCTGTGATCGTCGGCAGGTACAA | 38 |
| TBX1 | GTGAAGAAGAACGCGAAGGT | 39 | TGGTGACGATCATCTCGGT | 40 |
| FGF10 | TGTGGAAATGGATACTGACACA | 41 | GACGGAAGACACCAAGAACA | 42 |
| HAND1 | GCTGGAGTTAAACCAGTGA | 43 | GATGCGTAGCACCAGTC | 44 |
| HAND2 | GCGTGAATGTTCCCTCTT | 45 | CTAGTGCCCACTGTCTTTATC | 46 |
| NR2F2 | TGATGTAGCCCATGTGGAAAG | 47 | GCTGCCGGACAGTAACATATC | 48 |
| ALDH1A2 | TTTGCCAAGTTCCATTGTGCCAGG | 49 | TGGTGGAGTCACTGGAAAGCAGAA | 50 |
| CXCR4 | AGGGAACTGAACATTCCAGAGCGT | 51 | TTT GTA CAC CAA GCA CCT ATT T | 52 |
| TDGF1 | AGGAATTTGCTCGTCCATCTC | 53 | AAGACCGAGGCCGAATTG | 54 |
| PITX2 | AGACTGAAAGCAAAGCAGCACTCC | 55 | ATACTGGCAAGCACTCAGGTTGGA | 56 |
| GSC | ACGATGCTACTTTCTTGCACACGC | 57 | ACCCTCCCGGCTCTGTACACTATTTA | 58 |
| FGF17 | TCCGCGAGTACCAACTCTA | 59 | CAAACTTGTTGCCGTCCTC | 60 |
| BMP4 | TCAGGCAGTCCTTGAGGATAGACA | 61 | AAGCAGTCTGTGTAGTGTGTGGGT | 62 |
| GATA6 | GAGAACAGCGAGCTCAAGTAT | 63 | CACGGAGGACGTGACTT | 64 |
| GYPB | CACCTGCTGTTCTCTTGTTTATG | 65 | CAGTAATAGTGAGGCAGGAGAAC | 66 |
| GAL | TGGCAACCACAGGTCATT | 67 | CTGGTTTCATGTCATCTTCGG | 68 |
| PCDH19 | CTGACCTCCTCCCTCAACTATT | 69 | GGGTATTCTGGTTCTCCACATTC | 70 |
| PRICKLE1 | GATCAAGAGACTCCCGAAGAAG | 71 | GTAGTTCAGAGCAGGCGATAAG | 72 |
| HES7 | GCTGCTACTTGTCCGGTTT | 73 | CGCAGATAGCCGTGCAG | 74 |
| RBP1 | CAACTGGCTCCAGTCACTC | 75 | GCAGGTACTCCTCGAAATTCT | 76 |
| CRABP1 | AACGCCATGCTGAGGAAA | 77 | CGTGGTGGATGTCTTGATGT | 78 |
| HOTAIRM1 | GCCAGAAACCAGCCATAGT | 79 | CATAGGTTTGCTCCCTACCTTC | 80 |
| PHLDA1 | TGTACTTCACTGTGGTGATGG | 81 | GATGGCCTGACGATTCTTGTA | 82 |
| CYP26A1 | CCAAGGGCTGGAATGTTATCT | 83 | GGAGATTGTCCACAGGATACAC | 84 |
| CD1D | CCTGGGAACGCCTCAAATAA | 85 | GAATGGCCAAGTTTACCCAAAG | 86 |
| ITGA3 | CAGACCTACCACAACGAGATG | 87 | ACACAGTGTTCTGGGTGAAG | 88 |
| NR2F1 | TACCTGTCCGGCTACATCTC | 89 | CAGATGTTCTCGATGCCCATAA | 90 |
| SIX1 | TGCCGTCGTTTGGCTTTA | 91 | CTTGAGTACGCTCTCGTTCTTG | 92 |
| UNC45B | CGGACATCAAGGCTCTGTATC | 93 | CATCTCCTGGAAGTTCTGGTTC | 94 |
| IRX4 | TTGGACTCCTGGGAACATGGACAA | 95 | ATGCTTCAGGGTATCTGGCCTCTT | 96 |
| PDGFRB | TGGGCTAGACACGGGAGAATACTT | 97 | AAGATGTAGAGCCGTTTCCGCTCA | 98 |
| IRX1 | GACTGTACACCTGCCACATC | 99 | CAGGAAGGAGCGCATGTT | 100 |
| MEIS2 | GGTTCACCACCTGAACCAC | 101 | TGACAGCGGATCCCATACT | 102 |
| MYOZ1 | GCTGGTCAGGGATTCTCATAC | 103 | CTGCTGATCAGAGCCATACTG | 104 |
| IRX2 | GCTAGCCATCATCACCAAGAT | 105 | CCAGGTCATCTTGTTCTCCTTC | 106 |
| TXLNB | GTCCAACCACCAGAGAAAGAG | 107 | GCAGCAGACCCATAAGTGTTA | 108 |
| COX7A1 | GCTGATCCGCTCCTTCAG | 109 | TGGAAGAGCTTCTGTTTCTCG | 110 |
| PLN | ACCATTGAAATGCCTCAACAAG | 111 | ACGATGATACAGATCAGCAAGAG | 112 |
| TBX20 | GCTCCTGGGTATCATCTTCT | 113 | GAGGAATGGGTGTTGCTATG | 114 |
| TMEM88 | CTGTTCTTGTAACAGCCCAGAA | 115 | TAGCAAGATGGTCCCTAGCA | 116 |
| TBX18 | TTAACCTTGTCCGTCTGCCTGAGT | 117 | GTAATGGGCTTTGGCCTTTGCACT | 118 |
| DKK1 | TCTTTGTCGCGATGGTAGC | 119 | GGTTCTTGATAGCGTTGGAATTG | 120 |
| TBP | TGAGTTGCTCATACCGTGCTGCTA | 121 | CCCTCAAACCAACTTGTCAACAGC | 122 |

### Example 5—Immunocytochemistry and Imaging

EBs were fixed by 4% paraformaldehyde, embedded, and sectioned. After the deparaffinization and rehydration, heat-induced epitope retrieval was performed followed by immunostaining. The following antibodies were used for immunostaining: mouse anti-cardiac isoform of cTNT (ThermoFisher Scientific, 1:200), rabbit anti-human cTNT (abcam, 1:200), rabbit anti-CX43 (abcam, 1:800), rabbit anti-TBX5 (ThermoFisher, 1:200), rabbit anti-HAND1 (LSBio, 1:200), rabbit anti-HEY2 (Proteintech, 1:200), mouse anti-NR2F2 (Bio-Techne, 1:200), mouse anti-NR2F1 (Bio-Techne, 1:200), rabbit anti-IRX1 (ThermoFisher Scientific, 1:200). For detecting unconjugated primary antibodies, the following secondary antibodies were used: donkey anti-mouse IgG-Alexa488 (ThermoFisher, 1:500), donkey anti-rabbit IgG-Alexa488 (ThermoFisher, 1:500), donkey anti-mouse IgG-Alexa555 (ThermoFisher, 1:500), or donkey anti-rabbit IgG-Alexa555 (ThermoFisher, 1:500). IRX1 and CX43 expression was measured by counting the number of CX43+staining in one field of view (×40 magnification) in cTNT+cardiomyocytes randomly selected from 1 area in each EB. Data were collected from 3 independent experiments. All imaging was taken by Zeiss LSM700 confocal microscope.

### Example 6—Quantification and Statistical Analysis

Standard statistical analyses were performed using GraphPad Prism 8. The number of replicates, type of statistical test and test result are described in the Detailed Description of the Drawings. All data are represented as mean±standard error of mean (SEM). Statistical significance of two-group comparison was determined by unpaired student's t test and that of three or more groups was determined by one-way ANOVA analysis with Bonferroni post-hoc test in GraphPad Prism 8 software. Results are significant at p<0.05 (*), p<0.01 (**), p<0.001 (***), p<0.0001 (****). Sample size of all the experiments was not pre-determined, and no randomization or investigator blinding approaches were implemented during the experiments and data analyses given the natures of the study.

### Example 7—Sample Preparation, Single-Cell Library Generation and Raw Data Processing

Mesoderm (day 3), late mesoderm (day 4), progenitor (day 6) and myocyte (day 20) populations were generated from HES2 hPSC line. Cells were dissociated to single cells as described above and stained with DAPI. Live cells were then sorted using FACSAria Fusion (BD) at the Sick Kids/UHN flow cytometry facility. Single-cell libraries from these cell suspensions were generated in the 10× Genomics Chromium controller using Chromium Single Cell 3′ Reagent Kit v3. FHF, aSHF and pSHF cells of the same developmental stage/differentiation phase were sequenced together and all libraries were sequenced simultaneously. Chromium Single Cell Software Suite v3 was used for processing the single cell RNA-seq raw data produced in the 10× Chromium Platform, which includes sample demultiplexing, read alignment, barcode processing, and UMI counting. “Cellranger mkfastq” was used to generate FASTQ files from BCL files. Next, “cellranger count” was used to generate single-cell gene counts for a single library. Reads in the FASTQ files were mapped to the human reference genome (NCBI build38/UCSC hg38) with STAR software. Reads were confidently mapped to the exonic loci with MAPQ 255. Chromium cellular barcodes were used to generate gene-barcode matrices. Only reads that were confidently (uniquely) mapped to the transcriptome were used for the UMI count. Filtered gene-barcode matrices containing only cellular barcodes were used for downstream analyses.

### Example 8—Cell Filtering and Cell-Type Clustering Analysis

The transcriptomes of 2672 day 3 FHF cells, 5228 day 3 SHF cells, 4108 day 4 FHF cells, 8606 day 4 SHF cells, 3146 day 6 FHF cells, 3241 day 6 aSHF cells, 3231 day 6 pSHF cells, 2352 day 20 FHF cells, 2909 day 20 aSHF cells and 3535 day 20 pSHF cells were captured and sequenced. Prior to the downstream analyses, raw data of cells of the same stage and various lineages (FHF, aSHF and pSHF) were aggregated as a single object. Further filtering of low-quality cells and doublets, regressing of mitochondrial and cell cycle factors and clustering analyses of these cells were performed with the Seurat v.3.2.2 R package (Macosko et al., 2015, Cell, 161:1202-14; Satija et al., 2015, Nat. Biotechnol., 33:495-502). For the analyses of hPSC datasets, filtered cells were analyzed using the ‘SCTransform’ pipeline before principal component analysis (Hafemeister & Satija, 2019, Genome Biol., 20:296), whereas aggregated mouse datasets were normalized for genes expressed per cell and total expression, then multiplied by a scale factor of 10,000 and log-transformed (NormalizeData and ScaleData functions) using the standard Seurat pipeline. Following data normalization and scaling, significant principal components were calculated (RunPCA function) and the top 25 PCAs were used for downstream graph-based, semi-unsupervised clustering into distinct populations (FindClusters function) and uniform manifold approximation and projection (UMAP) dimensionality reduction was used to project these cells in two dimensions (RunUMAP function). For clustering, the resolution parameter was approximated based on the number of cells and distinct marker expressed in discernible clusters. Specifically, resolutions from 0.4 to 0.8 were used for aggregated datasets in the present study (0.4 for days 3, 4, 6 data and 0.8 for day 20 data). To identify marker genes or upregulated genes, the clusters of interest were subset and compared for differential gene expression using the Wilcoxon rank-sum test (FindAllMarkers function; only.pos=TRUE, min.pct=0.1, logfc.threshold=0.1, p-value cut-off=0.05).

### Example 9—Batch Correction, Integration of Multiple Datasets and Correlation Analysis

Batch effects in hPSC-derived cells of multiple lineages and stages used for downstream pseudotime analyses were corrected by matching mutual nearest neighbors (mnn) with the package batchelor (Haghverdi et al., 2018, Nat. Biotechnol., 36:421-7) and Seurat wrapper. The corrected data were then transformed to appropriate formats for downstream analyses (single cell experiment object for slingshot analysis). The integration of human fetal and hPSC data was achieved by canonical correlation analysis (CCA) provided in the Seurat package (Stuart et al., 2019, Cell, 177:1888-1902). Correlation between human fetal and hPSC-derived cells was estimated with Pearson correlation based on the mean expression values of 3000 variable genes stored in the integrated data after CCA correction. The resulting Pearson correlation scores were visualized with the package ggcorrplot following hierarchical clustering.

### Example 10—Gene Ontology, Gene Set Enrichment Analyses and Human to Mouse Gene Transformation

Gene ontology and gene set enrichment analyses were performed with the ClusterProfiler, MSigDB, org. Hs.eg.db and org.Mm.eg.db packages (Carlson et al., 2016, Genomic Annotation Resources in R/Bioconductor. In Statistical Genomics: Methods and Protocols, Mathé and Davis, eds. (New York, NY: Springer), pp. 67-90; Subramanian et al., 2005, PNAS USA, 102:15545-50; Yu et al., 2012, Omics J. Integr. Biol., 16:284-7). Upregulated genes in the clusters of interest were obtained using the FindAllMarkers function provided in the Seurat package (only.pos=TRUE, min.pct=0.1, logfc.threshold=0.1, p-value cut-off=0.05). The gene symbols were transformed to Entrez Gene IDs using the annotations provided in the org. Hs.eg.db package. Note that org. Mm.eg.db package was used for mouse data analysis. In the cases where transformation between human and mouse genes were required (analysis of human and mouse conserved genes), the useMart and getLDS functions provided in the biomaRt package were used (Durinck et al., 2005, Bioinforma. Oxf. Engl., 21:3439-40; Durinck et al., 2009, Nat. Protoc., 4:1184-91). The genes enriched in these clusters were subsequently analyzed for the enrichment of biological processes (BP) using the compareCluster function (function=enrichGO, ontology=BP, pvalueCutoff=0.05) with the package ClusterProfiler. To generate dot plots, the analyzed data (containing gene number, adjusted p values, cluster identities and others) were retrieved and visualized with the package ggplot2. For GSEA analysis, all the genes in a given cluster were retrieved and the ranked by their expression level (LogFC). With the sorted/ranked data frame, KEGG and GSEA analyses were performed with the packages ClusterProfiler and MSigDB. Finally, the results were visualized with the package enrichplot.

### Example 11—Pseudotime Trajectory Analysis

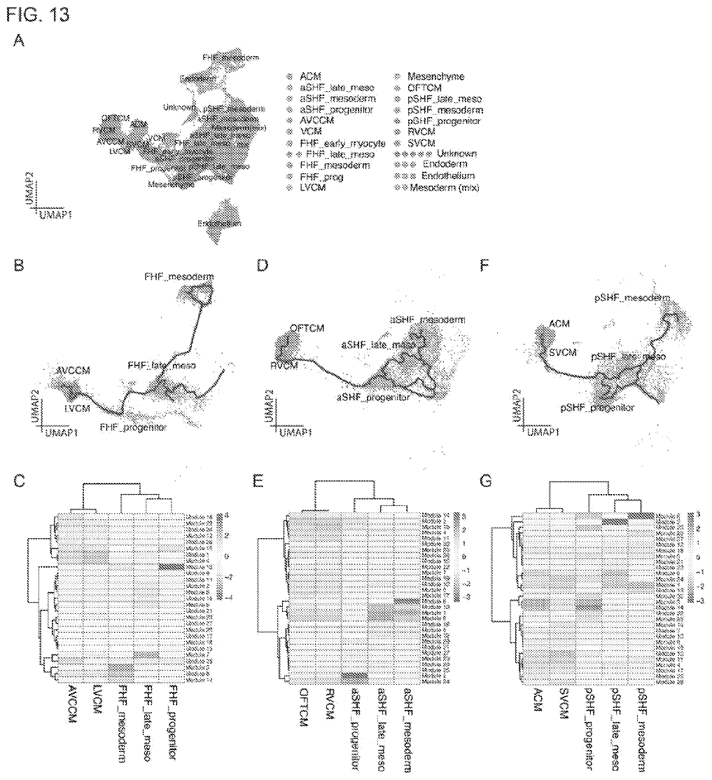

Pseudotime and cell trajectory analyses were performed with the Monocle3 and slingshot packages as described in the papers and tutorials (Qiu et al., 2017, Nat. Methods, 14:979-82; Street et al., 2018, BMC Genomics, 19:477; Trapnell et al., 2014, Nat. Biotechnol., 32:381-6). The analysis of integrated hPSC-derived mesoderm and human gastrulation dataset was performed with slingshot. The processed Seurat object was transformed to a single cell experiment object, which was then analyzed with the slingshot package (slingshot and getLineages functions). Note that the start cluster was pre-determined for the slingshot analysis (epiblast for the integrated mesoderm data). Monocle3 package was used to perform pseudotime inference for the aggregated hPSC data shown in FIG. 13 given that this package provides a function (find_gene_modules function; resolution=1e-3, reduction_method=c (“UMAP”)) that reveals upregulated gene modules as cells transition through different stages. For these analyses, different lineages (FHF, aSHF and pSHF) that encompass all the developmental stages described in this paper were transformed to CellDataSet objects (as.cell_data_set function) and analyzed separately (learn_graph, plot_cells and order_cells functions). The resulting pseudotime values of all the cell types were added to the meta data of their corresponding Seurat objects for downstream visualization.

### Example 12—Generation of FHF and SHF Cardiac Mesoderm Populations from hPSCs

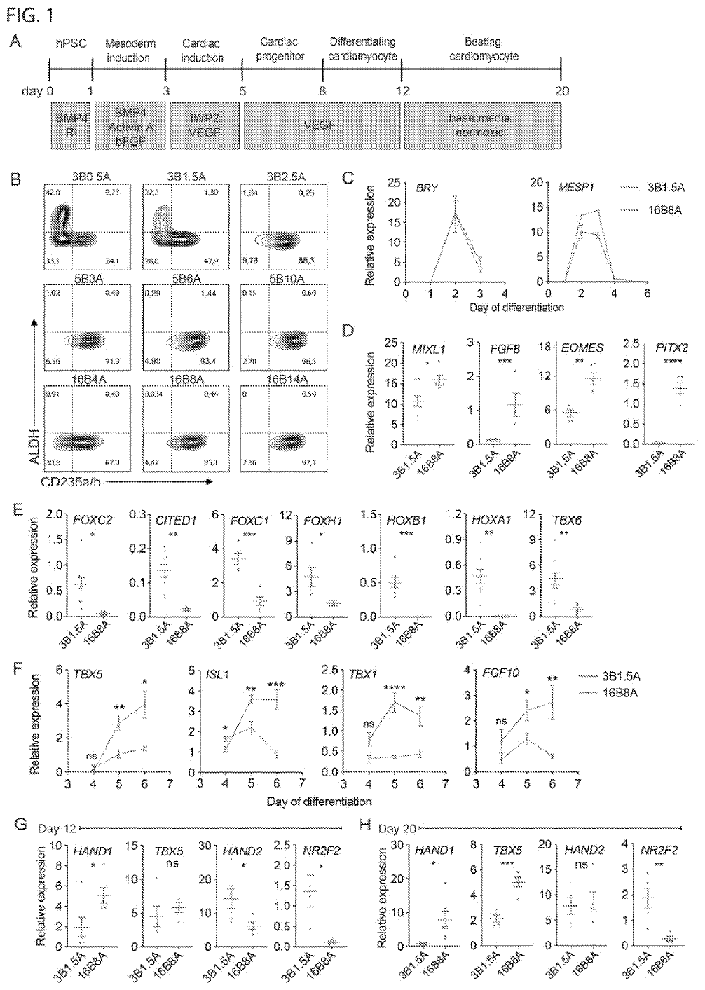

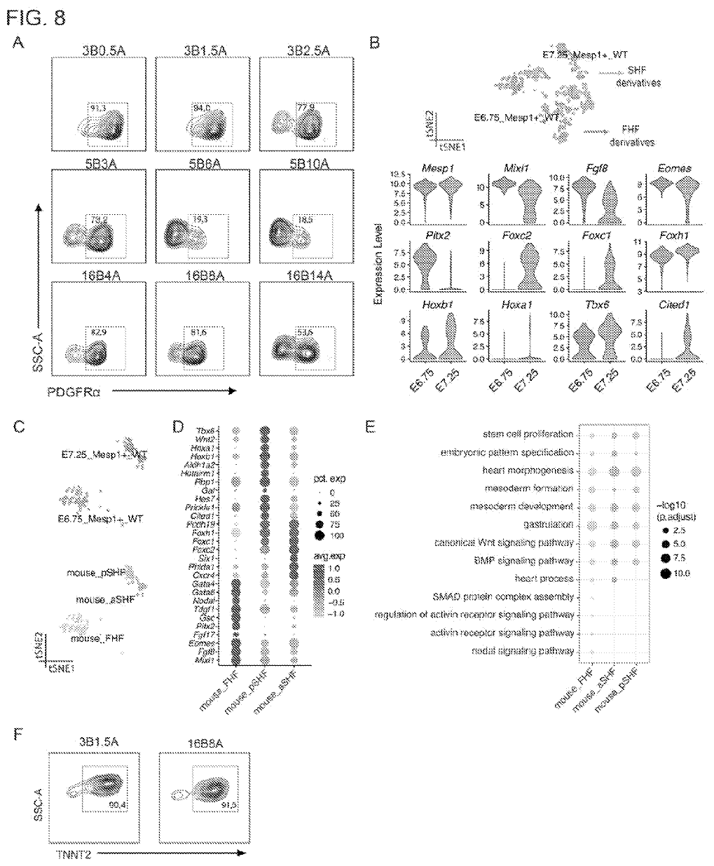

Previous findings that distinct ventricular and atrial mesoderm populations can be induced through the staged manipulation of BMP and Activin A signaling suggested that a similar strategy could be used to segregate the FHF and SHF fates at this early stage of development. To test this, Activin A and BMP4 concentrations were varied between days 1 and 3 of differentiation to induce mesoderm subsets that display the defining features of populations with FHF and SHF potential (FIG. 1A). At day 4, mesoderm induction was monitored by expression of PDGFR-alpha together with either CD235a/b or ALDH1A2, markers that have previously been shown to track with the induction of ventricular and atrial mesoderm, respectively (FIGS. 1B and 8A). For these analyses, the Aldefluor (ALDH) assay was used as a measure of ALDH1A2 activity (Jones et al., 1995, Blood, 85:2742-6).

As observed in previous studies, most of the cells in the mesoderm populations generated with high concentrations of BMP4 and Activin A (over 5 ng/ml of BMP4 and 3 ng/ml of Activin A) expressed CD235a/b and lacked ALDH activity (FIG. 1B). In contrast, mesoderm specified by lower concentrations of BMP4 and Activin A (3 ng/ml BMP4 and 0.5-1.5 ng/mL Activin A) contained significantly more ALDH⁺ cells and fewer CD235a/b⁺ cells than the populations induced with high concentrations of cytokines (FIG. 1B). Notably, small changes in the concentration of Activin A dramatically impacted the proportion of ALDH⁺ cells induced. Comparison between populations generated using 3B1.5A, 5B6A, 16B4A and 16B8A showed that 16B8A-induced mesoderm contained the highest frequency of PDGFR-alpha⁺ CD235a/b⁺ cells, whereas 3B1.5A-induced mesoderm contained ALDH⁺ and CD235a/b⁺ cells (FIGS. 1B and 8A). These findings are consistent with previous studies and show that proper levels and ratios of BMP4 and Activin A are required for inducing different mesoderm populations.

Molecular analyses of the populations induced with 3B1.5A and 16B8A showed similar kinetics of brachyury (T) and MESP1 expression, indicating that the temporal development of the primitive streak-like (brachyury⁺) and mesoderm (MESP1⁺*) populations was not impacted by the different concentrations of cytokines (FIG. 1C). To determine if the two hPSC-derived mesoderm subsets share similarities to the mouse FHF and SHF mesoderm identified previously (Lescroart et al., 2014, Nat. Cell Biol., 16:829-40; Lescroart et al., 2018, Science, 359:1177-81), the two hPSC-derived mesoderm subsets were analyzed for expression of genes that distinguish the mouse populations, identified through re-analyses of the Lescroart data. These analyses showed that the early emerging (E6.75) mesoderm expressed higher levels of Mixl1, Fgf8, Eomes and Pitx2, whereas Foxc1, Foxc2, Foxh1, Hoxb1, Cited1, Hoxa1 and Tbx6 were found at higher levels in the later emerging (E7.25) mesoderm (FIG. 8B). In addition to these differences, more detailed analyses provided further resolution and showed that the early emerging population expressed elevated levels of additional genes associated with the formation of FHF-like mesoderm, whereas the later population contained distinct subpopulations that showed molecular profiles of aSHF and pSHF mesoderm (FIGS. 8C and 8D). Gene ontology (GO) analysis of the genes upregulated in these mesoderm subtypes revealed an enrichment of Nodal/Activin signaling in the FHF mesoderm (FIG. 8E), supporting the requirement of high Nodal concentration for the generation of this mesoderm type in mouse embryo. RT-qPCR analyses of the two hPSC-derived populations identified above showed that the FHF genes (MIXL1, FGF8, EOMES and PITX2) were expressed at higher levels in the 16B8A-induced mesoderm, whereas the levels of genes associated with SHF mesoderm including FOXC2, CITED1, FOXC1, FOXH1, HOXB1, HOXA1 and TBX6 were significantly higher in the 3B1.5A mesoderm (FIGS. 1D and 1E).

To evaluate the potential of the two hPSC-derived mesoderm populations with respect to FHF and SHF potential, the cells were cultured for additional periods of time under cardiogenic conditions and the resulting populations analyzed for expression of genes indicative of the two heart fields. At days 5 and 6 of differentiation, the stage at which cardiovascular progenitors are specified, the cells generated from the 16B8A-induced mesoderm expressed higher levels of the FHF progenitor marker TBX5 and lower levels of the SHF progenitor markers FGF10, TBX1 and ISL1 than the derivatives of the 3B1.5A-induced mesoderm (FIG. 1F). While both populations efficiently generated cardiomyocytes (FIG. 8F), they displayed differential gene expression patterns at days 12 and/or 20 of differentiation. Cardiomyocytes generated from the 16B8A induction expressed higher levels of the left ventricular (FHF) genes HAND1 and TBX5 than those derived from the 3B1.5A induction (FIG. 1H). In contrast, expression of HAND2 (day 12) associated with right ventricular (SHF) development and NR2F2 indicative of atrial (SHF) differentiation were expressed at higher levels in the 3B1.5A-derived cardiomyocytes (FIGS. 1G and 1H). Taken together, these findings support the interpretation that the 16B8A-induced population represents FHF mesoderm, whereas the one induced with 3B1.5A displays molecular profiles and developmental potential of SHF mesoderm.

### Example 13—Characterization of Heterogeneity and Temporal Specification of hPSC-Derived Cardiac Mesoderm Populations

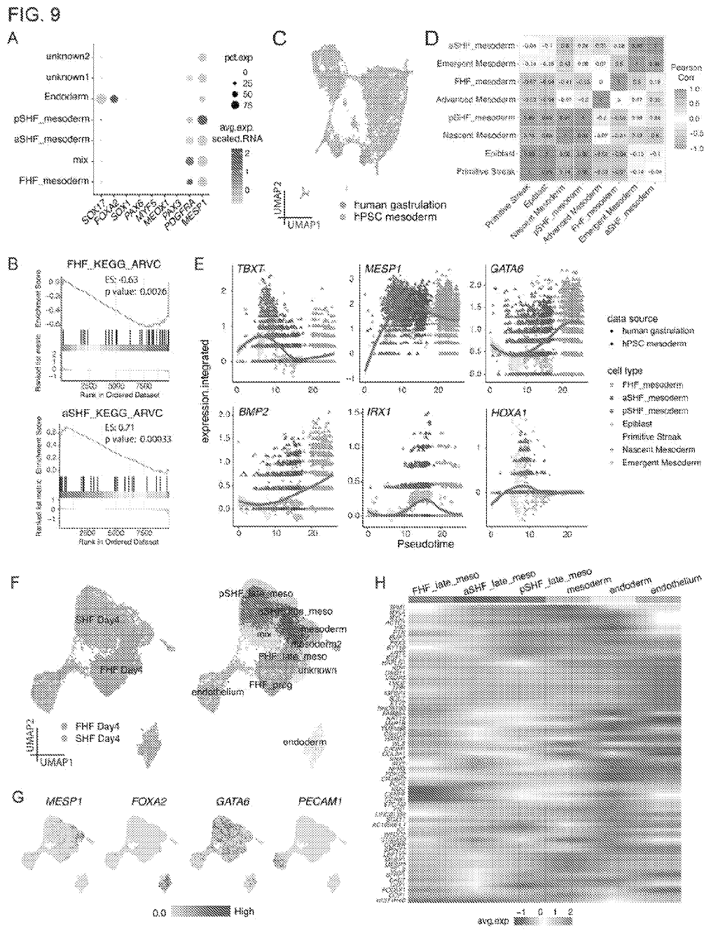

To further investigate the molecular characteristics of the 3B1.5A- and 16B8A-induced mesoderm populations, scRNA-seq was carried out at day 3 of differentiation. Analyses of combined data sets revealed the presence of 7 distinct clusters that included 6 PDGFRA⁺MESP1⁺ mesoderm clusters and an endoderm cluster identified by the expression FOXA2 and SOX17 (FIGS. 2A and 9A). The 16B8A-induced population consisted of a single mesoderm cluster, while that induced with 3B1.5A resolved into 3 main clusters (FIG. 2A). To annotate the different clusters, expression was analyzed for FHF as well as for both aSHF, and pSHF mesoderm markers obtained from the analysis of Mesp1′ E6.75 and E7.5 mouse mesoderm (FIG. 8D). As expected from the initial analyses, the 16B8A-induced population expressed the highest levels of EOMES and LHX1, indicating it represents FHF mesoderm (FIG. 2B and Appendix A). Within the 3B1.5A-induced mesoderm, a putative aSHF cluster was identified that expressed SIX1 and FOXC2, a putative pSHF cluster that expressed HOXA1 and ALDH1A2, as well as an additional cluster that co-expressed aSHF and pSHF mesoderm markers (FIG. 2B and Appendix A). None of the clusters expressed genes indicative of the presence of paraxial mesoderm (PAX3, MEOX1 and MYF5) or neuroectoderm (PAX6 and SOX1), consistent with the interpretation that they represent lateral plate mesoderm (FIG. 9A).

We next sought to identify the molecular features of each mesoderm subtype that are conserved between human and mouse by performing differential expression analyses on the human and mouse FHF, aSHF, and pSHF mesoderm clusters. Overall, 382 differentially expressed genes (DEGs) (219 pSHF genes, 60 aSHF genes and 103 FHF genes) were detected that showed conserved cross-species patterns in the different mesoderm subtypes (Appendix A and FIG. 2C). The list contained some of the known genes that distinguish these mesoderm populations as well as many others that have not been previously described as showing differential expression patterns between them (Appendix A and FIG. 2D). For example, it was found that PITX2 and GSC, conventionally used as primitive streak laterality and polarity markers, are preferentially expressed in the FHF-like mesoderm, whereas HOTAIRM1, a long non-coding RNA located in the HOXA gene cluster between HOXA1 and HOXA2, is expressed at the highest levels in pSHF-like mesoderm. RT-qPCR analyses confirmed some of the differential expression patterns and showed that the 16B8A-induced mesoderm expressed higher levels of the newly identified FHF genes including TDGF1, LHX1, GSC, FGF17, BMP4, BMP2, GATA6 and GYPB (FIG. 2E) than the 3B1.5A-induced mesoderm. By contrast, the pSHF markers GAL, PCDH19, ALDH1A2, PRICKLE1, HES7, RBP1, CRABP1 and HOTAIRM1 (FIG. 2F), as well as the aSHF markers PHLDA1 and CXCR4 (FIG. 2G), identified in these analyses were detected at significantly higher levels in the 3B1.5A-induced mesoderm. The findings from these different molecular analyses further support the interpretation that 16B8A-induced population represents FHF mesoderm whereas the one induced with 3B1.5A contains both aSHF and pSHF mesoderm.

To gain insights into pathways involved in regulating biological dynamics within distinct mesoderm populations, knowledge base-driven enrichment analyses was performed with the pathway annotations from publicly available databases. Interestingly, GO analysis of the species conserved DEGs of each population indicated an enrichment of pattern specification process and regionalization in all the clusters, suggesting that these genes may reflect the spatiotemporal dynamics observed within the primitive streak during gastrulation (FIG. 2H). Terms relevant to BMP signaling and cardiac muscle development were mainly enriched in the FHF mesoderm (FIG. 2H), suggesting that these cells are committed to the cardiomyocyte lineage through early onset of BMP signaling (Lescroart et al., 2018, Science, 359:1177-81). Components of the Wnt signaling pathway and retinoic metabolic processes were enriched in the pSHF clusters (FIG. 2H), an assignment consistent with the known roles of these pathways in pSHF development in the mouse. Similarly, the gene set involved in outflow tract morphogenesis was enriched in the aSHF cluster (FIG. 2H), the origin of outflow tract cardiomyocytes in the mouse. Gene set enrichment analysis (GSEA) with KEGG pathway annotations revealed an enrichment for genes annotated to arrhythmogenic right ventricular cardiomyopathy (ARVC) in the aSHF mesoderm (FIG. 9B). As ARVC primarily affects the right ventricle, this observation supports the interpretation that the aSHF mesoderm is the source of RVCMs in the human.

To determine if the hPSC-derived FHF, aSHF and pSHF populations represent temporally distinct stages of mesoderm development as observed in the mouse, our data was integrated with scRNA-seq data from a human gastrulating embryo that contained temporally distinct mesoderm subpopulations (nascent, emergent and advanced) (Tyser et al., 2020, Cold Spring Harbor Perspect. Biol., 12:a037135). These integration analyses revealed a significant overlap between the hPSC- and human embryo-derived data (FIGS. 2I and 9C) as demonstrated by the co-clustering of the hPSC and embryonic endoderm, as well as that of the hPSC and embryonic mesoderm populations. Correlation and pseudotime analyses of the integrated data suggest that hPSC-derived FHF mesoderm represents the most advanced population whereas the pSHF mesoderm showed the highest correlation with embryonic nascent mesoderm (Pearson correlation score=0.91) that occupied the earliest pseudotime (FIGS. 2J and 9D). The aSHF mesoderm was positioned between the pSHF and FHF populations and correlated with the embryonic emergent mesoderm (Pearson correlation score=0.89) (FIGS. 2J and 9D). An enrichment of pSHF mesoderm markers in the nascent mesoderm and aSHF mesoderm markers in the emergent mesoderm was confirmed (FIG. 2K). As a reflection of the transition from primitive streak to mesoderm, analyses of the stage-specific markers along pseudotime suggested that the primitive streak marker TBXT was upregulated prior to the mesoderm marker MESP1, followed by the upregulation of GATA6 indicative of cardiac differentiation (FIG. 9E). Markers of FHF (BMP2), aSHF (IRX1) and pSHF (HOXA1) were expressed at distinct pseudotimes in a sequential order, supporting the temporal specificity of these mesoderm subtypes (FIG. 9E). Taken together, these findings support the interpretation that the hPSC-derived FHF, aSHF, and pSHF populations indeed represent temporally distinct mesoderm subsets that emerge in a pattern identical to that of their mouse counterparts.

In addition to the day 3 populations, scRNA-seq also was performed on day 4 populations induced with either FHF or SHF conditions. Analyses of these populations showed a marked reduction in the proportion of MESP1⁺ cells compared to day 3 and a high proportion of GATA6′ cells indicative of the specification of cardiac lineage. Populations of FOXA2⁺ endoderm and PECAM1⁺ endothelial cells were also detected (FIGS. 9F and 9G). More detailed analyses revealed the presence of a HAND1^{high} BMP4^{high} FHF cluster, a FOXC2^{high} TBX1^{high} aSHF cluster and a NR2F1^{high} HOXB1^{high} pSHF cluster (FIG. 9H). Based on these expression patterns, the day 4 population was considered to represent late-stage mesoderm, undergoing the initial specification steps to cardiac progenitors.

### Example 14—Generation of Distinct Cardiac Progenitors from Purified Mesoderm Populations

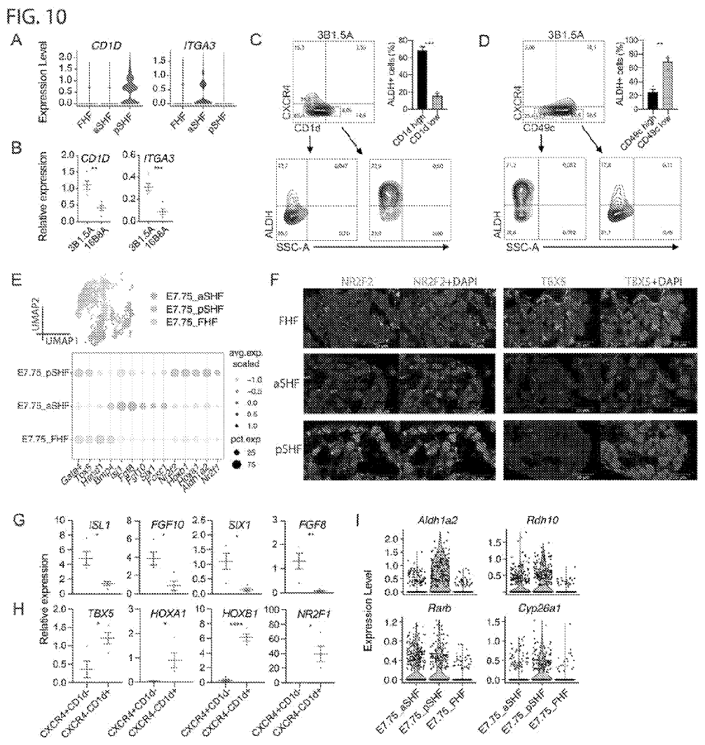

To be able to separate the FHF, aSHF and pSHF mesoderm populations for functional analyses, our data sets were queried to identify FACS-compatible markers that could be used to isolate these populations. Among the DEGs, it was found that CXCR4 and ITGA3 expression was highest in the aSHF cluster, CD1D and ALDH1A2 levels were highest in pSHF cluster and, as expected from our flow cytometric analyses, GYPB was preferentially expressed in the FHF cluster (FIG. 10A and Appendix A). RT-qPCR analyses further supported the differential expression of these markers in the 3B1.5A- and 16B8A-specified mesoderm cultures (FIGS. 2E-G and 10B). To determine if the expression of these FACS-compatible markers correlated with the expression patterns of the lineage-specific markers, we carried out Pearson correlation analyses. As shown in FIG. 3A, expression of GYPB positively correlated with the FHF mesoderm markers including DKK1, CYP26A1 and LHX1, whereas it negatively correlated with pSHF markers, such as RBP1 and TBX6. The expression of CXCR4 positively correlated with the aSHF markers FOXC1, FOXC2, PHLDA1 and TWIST1 and negatively with the FHF mesoderm marker TDGF1. Finally, ALDH1A2 expression positively correlated with the pSHF markers HOXA1, HOXB1, CDX2, and HOTAIRM1 and negatively with FHF markers LHX1, EOMES, CYP26A1 and TDGF1 (FIG. 3A).

Flow cytometric analyses of PDGFR-alpha⁺ mesoderm from day 4 3B1.5A- and 16B8A-induced populations were consistent with the assignment of these markers based on molecular profiling. The 16B8A-induced FHF population predominantly expressed CD235a/b but not CXCR4, and it did not show any ALDH activity (FIG. 3B). In contrast, the 3B1.5A-induced population could be segregated into a CXCR4⁺ fraction that lacked ALDH activity indicative of aSHF identity and a CXCR4⁻ fraction that contained ALDH⁺ cells reflective of pSHF mesoderm (FIG. 3C). Although CD235a/b protein was also expressed in the aSHF mesoderm, its transcript does not correlate with aSHF mesoderm markers. Using similar strategies, CD1D and ITGA3 (CD49c) were validated as novel cell-surface markers of pSHF and aSHF mesoderm respectively (FIGS. 10C and 10D).

To determine the potential of these different fractions, the cells were isolated by FACS at day 4 and cultured overnight as aggregates. The resulting populations were analyzed for expression of cardiac progenitor markers identified from analyses of E7.75 mouse FHF, aSHF and pSHF progenitors (FIG. 10E) as described in de Soysa et al. (2019, Nature, 572:120-4). As shown in FIG. 3D, the FHF mesoderm (ALDH⁻CD235a/b⁺CXCR4^{-/low})-derived population displayed the FHF progenitor signature identified in the mouse that includes expression of TBX5, HAND1, BMP4 and GATA4. The cells generated from the ALDH⁺CXCR4⁻ pSHF mesoderm expressed the pSHF markers NR2F2, HOXB1, HOXA1 and ALDH1A2 (FIG. 3E), whereas those derived from the ALDH CXCR4⁺ aSHF mesoderm expressed the aSHF markers FGF8, FGF10, FOXC1 and ISL1 (FIG. 3F). Given these expression patterns, the day 5 populations were considered as representative of the onset of the progenitor stage of development. In addition to transcriptomic analyses, higher expression levels of HAND1 and TBX5 protein were shown in the FHF progenitors and those of NR2F1 and NR2F2 protein in the pSHF progenitors by immunocytochemistry (FIGS. 3G and 10F).

To determine if CD1d could be used in place of ALDH as a marker for isolating pSHF mesoderm, the CD1d⁺CXCR4⁻ and CD1d⁻CXCR4⁺ fractions were isolated by FACS from day 4 SHF population, cultured for 24 hours and then the resulting populations analyzed by RT-qPCR for expression of pSHF and aSHF progenitor markers (FIGS. 10G and 10H). The CD1d⁺CXCR4⁻ mesoderm-derived population expressed the spectrum of genes indicative of pSHF progenitors, whereas the cells generated from the CD1d⁻CXCR4⁺ population displayed an expression pattern consistent with that of aSHF progenitors. Taken together, the findings from these cell fractionation studies demonstrate that it is possible to isolate day 4 mesoderm populations with FHF, aSHF and pSHF potential based on expression of CXCR4, GYPB, CD1d and the presence ALDH activity.

The expression of Aldh1a2 as well as other components of the RA signaling pathway including Rdh10, Rarb, and Cyp26a1 in the pSHF of the E7.75 mouse embryo is consistent with the known role of this pathway in maintaining the pSHF fate and in generating the derivative lineages in vivo (FIG. 10I). The expression of ALDH1A2 in the day 5 hPSC-derived pSHF population (FIG. 3E) suggests that RA signaling could play a similar role in the establishment and/or maintenance of the pSHF progenitor fate in the human. To test this, isolated day 4 pSHF cells (ALDH CXCR4⁻) were treated with either DMSO or retinol (2 μM) and the expression of cardiac progenitor markers examined 24 hours later. RT-qPCR analyses showed that treatment with ROH led to upregulation of the RA responsive gene CYP26A1, and the pSHF progenitor markers HOXA1 and HOXB1 (FIG. 3H). Interestingly, aSHF progenitor markers FGF10, FOXC1 and FOXC2 were downregulated as a result of ROH treatment (FIG. 3I). These findings indicate that RA signaling at this stage does function to enforce the pSHF molecular signature in the ALDH⁺CXCR4⁻-derived progenitor population.

### Example 15—Single Cell Transcriptomic Analyses of the Day 6 Progenitor Populations

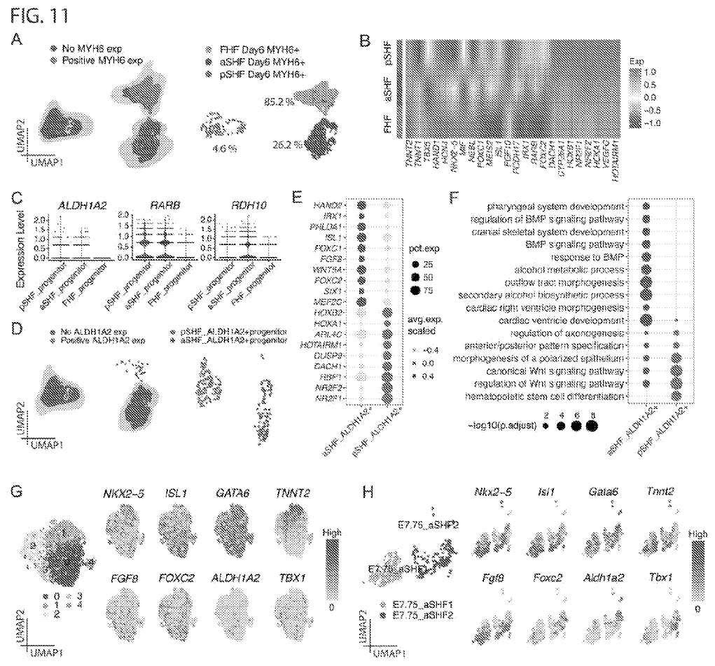

Following the generation of FHF, aSHF and pSHF progenitors, scRNA-seq was performed on these distinct populations (day 6) to investigate their cellular diversity and molecular profiles at the single cell level. Clustering analysis of all the populations identified clusters that represent FHF, aSHF, and pSHF lineages based on the expression of the lineage-specific markers: ISL1, HOXB1, NR2F2 and HOTAIRM1 for pSHF; FOXC1, FOXC2, FGF10 and WNT5A for aSHF; and TBX5, HAND1, HCN4 and MYH6 for FHF (FIGS. 4A and 4B). Expression of the cardiac myosin gene MYH6 in the majority (85%) of the FHF population indicates that these cells are undergoing differentiation towards cardiomyocytes. Significantly fewer MYH6′ cells were detected in the aSHF cluster (26%) and almost none were present the pSHF cluster (4.6%) (FIG. 11A). These differences recapitulate the temporal pattern observed at the onset of cardiogenesis in the mouse where the FHF lineage differentiates to cardiomyocytes prior to those of SHF lineages. Furthermore, analyses of these MYHG⁺ cells suggest that they retain lineage-specific markers expressed by their respective progenitors (FIG. 11B), highlighting the molecular differences between the earliest-emerging myocytes derived from distinct cardiac progenitors.

Having annotated hPSC-derived cardiac progenitors analogous to those in E7.75 mice, we then sought to identify markers of aSHF, pSHF and FHF progenitors that are conserved between humans and mice. Combined analyses of hPSC-derived progenitors and E7.75 mouse progenitors revealed 142 conserved FHF markers, 122 conserved aSHF markers and 97 conserved pSHF markers (FIGS. 4C and 4D; Appendix B). Among these markers, genes known to be differentially expressed in these populations were identified including WNT5A, SIX1, ISL1 and FGF10 in aSHF progenitors, DACH1, FOXF1, HOXB1 and NR22 in the pSHF progenitors and HAND1 in FHF progenitors. Additionally, expression patterns of genes were identified that have not been previously shown to differ between these progenitors, such as S100A10 and CSRP2 in FHF progenitors, RGS5, JAG1 and IRX genes in aSHF progenitors, as well as MEIS3 and CPE in pSHF progenitors (FIG. 4D and Appendix B).

Following the identification of species-conserved transcriptional features of FHF, aSHF and pSHF progenitors, the underlying signaling pathways involved in their development were investigated. GO analyses based on these markers showed that genes associated with cardiac ventricle development are enriched in the aSHF and FHF clusters (FIG. 4E), a finding consistent with the lineage tracing studies showing that LVCMs and RVCMs develop from FHF and aSHF progenitors respectively. Genes implicated in outflow tract morphogenesis and pharyngeal development were preferentially expressed in the aSHF progenitors, the progenitors that give rise to these cell types in the developing mouse heart. The pSHF cluster was enriched in genes involved in retinoic acid (RA) signaling, a pathway required for the specification of atrial cardiomyocytes from these progenitors (FIG. 4E).

While RA signaling is a well-established regulator of the pSHF-derived lineages, detail analyses revealed that the aSHF progenitors also express components of this pathway including RDH10, ALDH1A2 and RARB (FIG. 11C). Pseudotime ordering of the expression patterns of these genes in the day 3 mesoderm, day 4 late mesoderm and day 6 progenitor populations revealed different temporal expression patterns in the aSHF and pSHF lineages (FIGS. 4F and 4G). The pSHF lineage expressed ALDH1A2 and RDH10, from the day 3 mesoderm stage onward, whereas expression of these genes was not detected until day 4 of differentiation in the aSHF lineage (FIG. 4G). These patterns correlate well with ALDH activity, which was detected in more than 90% of the days 4 and 5 pSHF cells. By contrast, the day 4 aSHF cells were ALDH and less than 50% of the day 5 aSHF population showed this activity (FIGS. 4H, 4I and 3C). ALDH was not detected in the day 5 FHF population. Treatment of the day 5 aSHF cells with ROH for 24 hours led to an upregulation of the RA-responsive gene CYP26A1 as well as HOXB1 (FIG. 4J), known to be expressed in the IFT and OFT progenitors. Collectively, these findings show that pSHF and aSHF lineages upregulate the cellular machinery required for RA signaling at different developmental stages.

To further characterize the aSHF and pSHF ALDH1A2⁺ populations, DEG and GO analyses were performed to interrogate their transcriptomic differences. These analyses showed that the ALDH1A2⁺ pSHF cells express higher levels of the pSHF progenitor markers and lower levels of the aSHF progenitor markers than the ALDH1A2⁺ aSHF cells; the opposite pattern was found in the ALDH1A2⁺ aSHF cells (FIG. 11E). GO analysis based on the DEGs revealed that genes associated with outflow tract and right ventricle morphogenesis, pharyngeal system development and BMP signaling pathway are enriched in the ALDH1A2⁺ aSHF population (FIG. 11F). Re-clustering of the aSHF cells based on these observations revealed the presence of a large ALDH1A2′ cluster (cluster 0) that expressed the aSHF progenitor markers ISL1, FGF8, FOXC2 and TBX1. The aSHF cells also contained an ALDH1A2⁻ cluster (cluster 1) that expressed genes indicative of cardiomyocyte differentiation including TNNT2 and NKX-2.5 and lower levels of the progenitor markers (FIG. 11G), indicating the presence of cells undergoing differentiation to cardiomyocytes. Analyses of data from the E7.75 mouse embryo aSHF population identified a comparable Aldh1a2^{high} cluster (E7.75_aSHF2) that expressed aSHF progenitor markers (Tbx1 and Foxc2) as well as an Aldh1a2^{low} cluster (E7.75_aSHF1) that expressed genes associated with cardiomyocyte differentiation (FIG. 11H). Taken together, these findings strongly suggest that ALDH1A2 expression in the aSHF population marks the progenitor stage of lineage development.

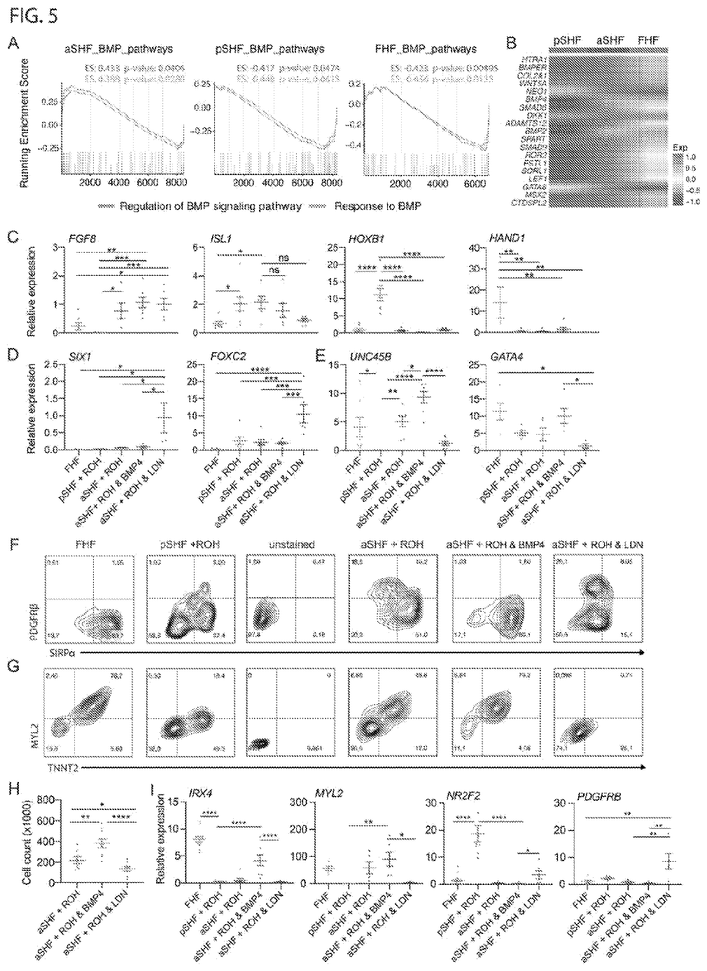

The above observation that the aSHF progenitors express genes involved in Bmp signaling is consistent with the known role of this pathway in the development of right ventricular cardiomyocytes from aSHF progenitors in the mouse. The presence of BMP signaling in the hPSC-derived aSHF lineage is further supported by GSEA analysis that showed an enrichment of ‘response to Bmp’ and ‘regulation of Bmp signaling pathway’ in the aSHF progenitors and a corresponding downregulation in pSHF and FHF progenitors (FIG. 5A). In line with this, it was found that the expression of specific genes involved in different aspects of Bmp signaling were upregulated in aSHF progenitors compare to the pSHF and FHF progenitors (FIG. 5B). The notable exception is the genes for the ligands BMP2 and BMP4, which are also highly expressed in the FHF lineage (FIG. 5B). To determine if BMP plays a role in the differentiation of the aSHF progenitors to derivative lineages, the day 5 cells were treated with either a pathway agonist (10 ng/mL BMP4) or antagonist (0.1 μM LDN) and then the population was analyzed 24 hours after the treatments. FHF and pSHF progenitors were included in the analyses, and ROH was added to the cultures of pSHF and aSHF progenitor, given that both populations display ALDH activity. As shown in FIG. 5C, manipulation of the BMP pathway did not alter the expression of genes indicative of aSHF (FGF8 and ISL1), pSHF (HOXB1) and FHF (HAND1) fates in the aSHF population. Inhibition of the pathway did, however, lead to upregulation of genes associated with pharyngeal progenitors (FOXC2 and SIX1) (FIG. 5D) and downregulation of those indicative of cardiomyocyte development (UNC45B and GATA4) (FIG. 5E). To further investigate the consequences of BMP manipulation, the agonist/antagonist treatment was extended for an additional 48 hours and then the treated cells were cultured for 12 days to promote cardiomyocyte development. FHF and pSHF progenitors also were cultured for 20 days without manipulation of BMP signaling. All 3 progenitor populations generated TNNT2⁺/SIRPa⁺ cardiomyocytes by day 20 of differentiation (FIGS. 5F and 5G). In addition to the cardiomyocytes, the aSHF-derived population also contained PDGFR-beta⁺ mesenchymal cells (FIG. 5F). Treatment with BMP4 increased the proportion and number of cardiomyocytes while reducing the number of the PDGFR-beta⁺ cells in the population, inhibition of the pathway had an opposite effect and increased the size of the PDGFR-beta⁺ fraction and reduced the proportion of cardiomyocytes (FIGS. 5F and 5H). Molecular analyses showed that both FHF- and aSHF-derived cardiomyocytes expressed the ventricular cardiomyocyte markers IRX4 and MYL2, whereas the cells generated from the pSHF progenitors expressed the atrial marker NR2F2 (FIG. 51). As expected from the flow cytometry analyses (FIGS. 5F and 5G), treatment of the aSHF cells with LDN resulted in a decrease in expression of the ventricular cardiomyocyte markers IRX4 and MY1.2 and elevated levels of PDGFRB (FIG. 51). Taken together, these findings show that the 3 different progenitors have cardiogenic potential and that the FHF and aSHF generate ventricular cells, whereas the pSHF gives rise to atrial cells. Additionally, they demonstrate that the generation of cardiomyocytes from the aSHF progenitors is dependent on BMP signaling.

### Example 16—Transcriptional Profiles of Cardiomyocytes Generated from hPSC-Derived FHF, aSHF and pSHF Progenitors

ScRNA-seq analyses of the day 20 cardiomyocyte populations generated from the FHF, aSHF and pSHF progenitors revealed developmental trajectories similar to those described in the mouse. Analyses of the clusters that express TNNT2 showed that the FHF progenitors gave rise to MYL2⁻HAND1^{high}IRX1^{low} LVCMs as well as to a population that displayed a gene signature of AVCCMs (MYL2⁺BMP2^{high}TBX2^{high}MSX2^{high}). The population derived from the aSHF progenitors contained a MYL2⁺HAND1⁻IRX1^{high} RVCM cluster as well as a MYL2^{low}HAND2⁺SEMA3C⁺ OFTCM cluster. The pSHF progenitors gave rise to NR2F2⁺TBX18⁻CAV1⁺NKX2-5⁺ ACMs and a SVCM/IFT cluster identified as a NR2F2⁺TBX18⁺CAV1⁻NKX2-5⁻ population (FIGS. 6A and 6B). In contrast to findings from mouse studies, ACMs were not detected in the hPSC FHF-derived population.

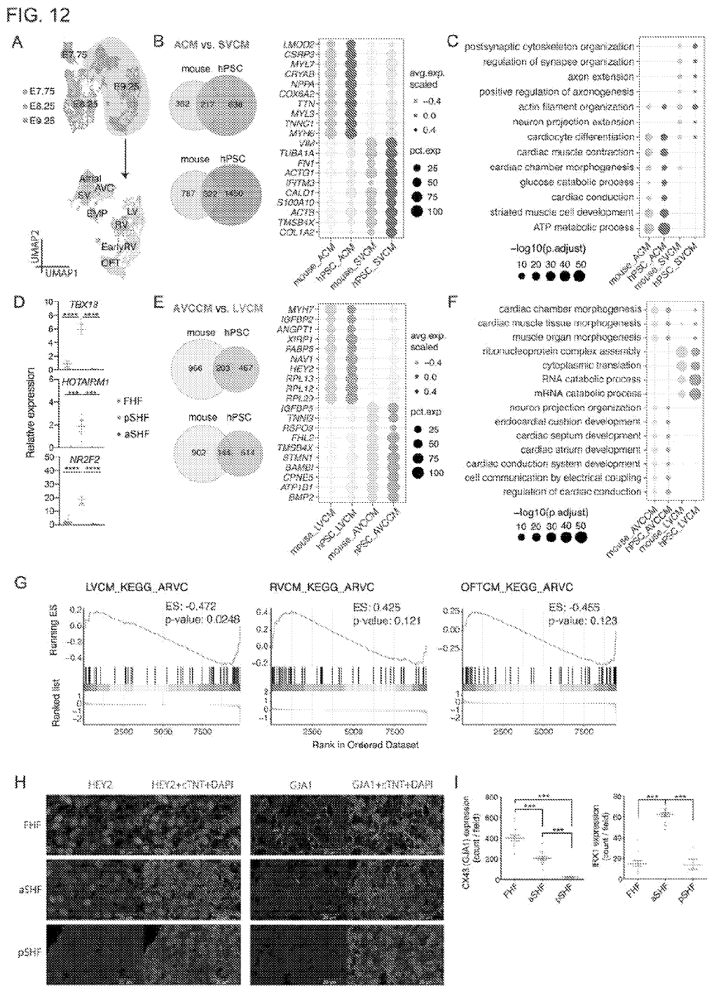

The data from the hPSC-derived cardiomyocytes was compared to published mouse E9.25 heart data (FIG. 12A) to identify conserved lineage-specific gene expression patterns for each of the CM subtypes. For these analyses, myocardium subtypes derived from the same heart field lineage were compared including ACM vs. SVCM (pSHF), RVCM vs. OFTCM (aSHF) and AVCCM vs. LVCM (FHF). Additionally, the putative LVCM and RVCM populations were compared. Analysis of the aSHF derivatives identified 112 RVCM markers (NPPB, MY1.2, PIN, IRX1 and others) and 153 OFTCM markers (CFC1, FHL1, SEMA3C and others) that are shared by humans and mice (FIG. 6C and Appendix C). GO analysis of these RVCM and OFT markers indicated that OFTCMs express elevated levels of genes related to outflow tract morphogenesis as well as those associated with transforming growth factor beta (TGF-β) production and responsiveness to TGF-beta. The link to a TGF-beta signaling is relevant as this pathway plays a pivotal role in endocardial cushion formation in the OFT region (FIG. 6D). As expected, RVCMs are enriched for genes associated with VCM development and functions including ventricle morphogenesis, sarcomere organization and muscle contraction (FIG. 6D). The comparison between mouse and hPSC-derived ACMs and SVCMs revealed 217 conserved ACM and 322 SVCM markers (FIG. 12B and Appendix C). This list includes previously reported mouse ACM (NPPA, KCNJ5 and NKX2-5) and SVCM markers (TBX18 and SFRP5) as well as genes that have not been shown to display differential expression between these populations (FIG. 12B and Appendix C). GO analysis indicated that ACMs express genes involved in cardiac muscle contraction and chamber morphogenesis, whereas SVCMs are enriched for axonogenesis and synapsis related genes (FIG. 12C). Analysis of the FHF derivatives identified 203 AVCCM and 144 LVCM conserved markers (FIG. 12E and Appendix C). GO analysis showed that LVCMs express genes involved in translation and transcription, whereas the AVCCMs express genes associated with endocardial cushion formation, septum development, cardiac conduction and electrical coupling (FIG. 12F), defining characteristics of AVCCMs. Finally, comparison between LVCMs and RVCMs revealed differential expression of known chamber-specific markers including HAND1 and TBX5 in LVCMs and IRX1, PLN and NPPB in RVCMs (FIG. 6E and Appendix C). This analysis also identified other genes that showed differential expression between the LVCMs and RVCMs. GO analysis based on the 260 LVCM and 127 RVCM markers indicated that RNA processing related genes are highly expressed in LVCMs whereas those associated with calcium signaling are preferentially expressed in RVCMs (FIG. 6F). Lastly, KEGG analyses of hPSC-derived RVCMs, OFTCMs and LVCMs demonstrated that the RVCMs are enriched for ARVC-associated genes, an assignment consistent with that of the day 3 aSHF mesoderm and further supports the interpretation that this population represent RVCMs (FIG. 12G).

To further verify the expression patterns of these genes, RT-qPCR and immunostaining analyses were carried out on the different CM subpopulations. As shown in FIGS. 6G and 6H, LV (FIG. 6H) and RV (FIG. 6G) genes identified in the above analyses showed differential expression between the FHF and aSHF-derived ventricular cardiomyocytes, strongly suggesting that they represent LV and RV cardiomyocytes respectively. Additionally, ACM and SV markers including TBX18, HOTAIRM1 and NR2F2 were highly expressed in the pSHF day 20 culture (FIG. 12D). Immunostaining analyses supported this lineage assignment, and showed that the FHF-derived cells expressed higher levels of HAND1, TBX5 and GJA1 protein than those generated from the aSHF progenitors; in contrast, the aSHF derivatives expressed higher levels of IRX1 (FIGS. 6I, 12H and 12I). HEY2, a compact ventricular myocyte marker, is only expressed in the FHF and aSHF derivatives (FIG. 12H).

To determine if these in vitro generated cardiomyocytes have similar molecular profiles to those in the human heart, the data from the day 20 hPSC-derived populations was integrated with a published human fetal heart dataset (Cui et al., 2019, Cell Rep., 26:1934-50) (FIG. 7A). This analysis revealed significant overlap between hPSC-derived VCMs and fetal VCMs, hPSC-derived ACMs and fetal ACMs and hPSC-derived mesenchyme and fetal valvular cells (FIG. 7A). The overlapping VCMs (NKX2-5⁺TNNT2⁺MYL2⁺) and pSHF CMs (TNNT2⁺NR2F2⁺) formed three distinct clusters 0, 1 and 3 (FIGS. 7A and 7B). Cluster 0 consists primarily of fetal and hPSC-derived ACMs, whereas clusters 1 and 3 represent fetal VCMs and hPSC-derived AVCCMs, LVCMs, OFTCMs and RVCMs (FIG. 7C). Correlation analysis of the cells in these three clusters confirmed transcriptional similarities between the hPSC-derived and fetal heart ACMs and VCMs (FIG. 7D).

### Example 17—Pseudotime Analyses of FHF, aSHE, and pSHF Development

Having characterized the transcriptomic profiles of mesoderm, late mesoderm, progenitor, and cardiomyocytes derived from FHF, aSHF, and pSHF lineages, we next sought to characterize the gene expression dynamics as the cells transition through developmental stages by performing pseudotime inference for the annotated clusters that encompass a broad spectrum of cardiac lineages (FIGS. 13A and 7E). As expected, day 3 mesoderm occupies the earliest pseudotime, followed by day 4 late mesoderm and day 6 progenitors, and subsequently day 20 CMs. Notably, the cells from the different heart fields form complimentary trajectories representing FHF, aSHF and pSHF lineage development (FIGS. 7E, 13B, 13D and 13F). It was next investigated whether the in-silico trajectories captured known lineage- and stage-specific gene expression dynamics by analyzing the upregulated gene modules of all the cell types throughout differentiation. These analyses identified distinct gene modules as signatures of cells of different stages and lineages (FIGS. 13C, 13E and 13G). For example, module 6, the aggregate of genes enriched in the aSHF mesoderm, is comprised of MESP1 and aSHF mesoderm markers including FOXC1, CXCR4 and TWIST1; similarly, the modules corresponding to aSHF progenitors and myocytes consist of aSHF progenitor markers (SIX1, FOXC2, TBX1, FGF10 and FGF8) and aSHF-derived myocyte markers (IRX1, MYOZ1, SEMA3C, MYL2 and others) (FIG. 13E). Similar gene expression dynamics was observed in the FHF and pSHF trajectories (FIGS. 13C and 13G).

### Example 18—Development of the FHF, aSHF and pSHF Lineages from the HES3 Line

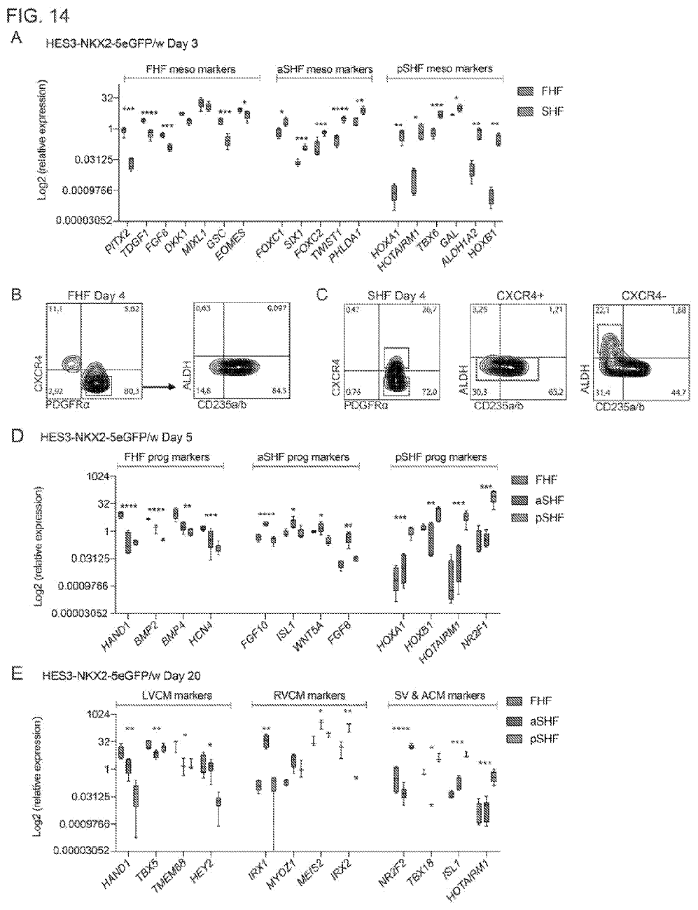

To determine if the approach for modeling FHF, aSHF, and pSHF development is applicable to other hPSC lines, the development of these lineages was investigated in populations generated from HES3-NKX2-5eGFP/w hESCs. Titration of BMP4 and Activin A concentrations between days 1 and 3 of differentiation revealed that 15B8A was optimal to induce a population that expressed FHF mesoderm markers, whereas 3B1A induced a mesoderm that expressed pSHF and aSHF genes (FIG. 14A). Flow cytometric analyses of the day 4 populations showed patterns almost identical to those observed with the HES2 cells. The majority of the FHF PDGFR-alpha⁺ population was CD235a/b⁺ and CXCR4^{-/low} ALDH⁻, whereas the SHF PDGFR-alpha⁺ population contained CXCR4⁺ALDH⁻ and CXCR4⁻ALDH⁺ fractions (FIGS. 14B and 14C). When isolated and cultured for 24 hours, the FHF mesoderm gave rise to cells that expressed markers indicative of FHF progenitors, whereas the CXCR4⁺ ALDH⁻ and CXCR4⁻ALDH⁺ fractions generated progeny that expressed genes indicative of aSHF and pSHF progenitors, respectively (FIG. 14D). Further culture of the FHF, aSHF and pSHF progenitors resulted in the development of cardiomyocytes from each population. Molecular analyses of the day 20 cardiomyocyte populations showed that those generated from the FHF progenitors expressed genes indicative of LVCM development, those from the aSHF progenitors expressed RVCM markers and those from the pSHF progenitors showed a profile indicative of ACM and SVCM (FIG. 14E). Collectively, these findings indicate that the strategy developed to model the development of FHF, aSHF and pSHF lineages can be translated to other hPSC lines.

It is to be understood that, while the methods and compositions of matter have been described herein in conjunction with a number of different aspects, the foregoing description of the various aspects is intended to illustrate and not limit the scope of the methods and compositions of matter. Other aspects, advantages, and modifications are within the scope of the following claims.

Disclosed are methods and compositions that can be used for, can be used in conjunction with, can be used in preparation for, or are products of the disclosed methods and compositions. These and other materials are disclosed herein, and it is understood that combinations, subsets, interactions, groups, etc. of these methods and compositions are disclosed. That is, while specific reference to each various individual and collective combinations and permutations of these compositions and methods may not be explicitly disclosed, each is specifically contemplated and described herein. For example, if a particular composition of matter or a particular method is disclosed and discussed and a number of compositions or methods are discussed, each and every combination and permutation of the compositions and the methods are specifically contemplated unless specifically indicated to the contrary. Likewise, any subset or combination of these is also specifically contemplated and disclosed.

| gene | cell type | p_val_adj | gene | cell type | p_val_adj | gene | cell type | p_val_adj |
|---|---|---|---|---|---|---|---|---|
| CER1 | FHF_mesoderm | 0 | PHLDA1 | aSHF_mesoderm | 1.07E−299 | CDX2 | pSHF_mesoderm | 0 |
| CYP26A1 | FHF_mesoderm | 0 | CRABP2 | aSHF_mesoderm | 6.29E−257 | POU5F1 | pSHF_mesoderm | 0 |
| LHX1 | FHF_mesoderm | 0 | COLEC12 | aSHF_mesoderm | 1.47E−173 | UCHL1 | pSHF_mesoderm | 1.72E−297 |
| TDGF1 | FHF_mesoderm | 0 | FBN2 | aSHF_mesoderm | 4.86E−132 | GAL | pSHF_mesoderm | 1.23E−279 |
| NPC2 | FHF_mesoderm | 0 | CDK6 | aSHF_mesoderm | 1.28E−116 | RSPO3 | pSHF_mesoderm | 1.06E−266 |
| TAOK3 | FHF_mesoderm | 0 | AHNAK | aSHF_mesoderm | 6.13E−116 | CSRP2 | pSHF_mesoderm | 1.36E−243 |
| L1TD1 | FHF_mesoderm | 0 | SFRP1 | aSHF_mesoderm | 3.88E−96 | TBXT | pSHF_mesoderm | 1.39E−241 |
| GYPB | FHF_mesoderm | 2.75E−301 | IRX1 | aSHF_mesoderm | 5.01E−84 | GPC3 | pSHF_mesoderm | 1.60E−235 |
| GYPE | FHF_mesoderm | 5.04E−288 | MN1 | aSHF_mesoderm | 1.67E−63 | HES7 | pSHF_mesoderm | 4.16E−235 |
| LRRC2 | FHF_mesoderm | 1.80E−284 | JCAD | aSHF_mesoderm | 3.57E−63 | LMO4 | pSHF_mesoderm | 6.33E−222 |
| GATA6 | FHF_mesoderm | 1.78E−260 | CXCR4 | aSHF_mesoderm | 4.18E−60 | PRICKLE1 | pSHF_mesoderm | 4.95E−214 |
| FGF17 | FHF_mesoderm | 1.91E−238 | GREB1L | aSHF_mesoderm | 5.39E−56 | WNT3A | pSHF_mesoderm | 1.06E−199 |
| COBLL1 | FHF_mesoderm | 5.79E−229 | JAG1 | aSHF_mesoderm | 1.37E−54 | TBX6 | pSHF_mesoderm | 1.71E−195 |
| GSC | FHF_mesoderm | 4.19E−211 | SIX1 | aSHF_mesoderm | 7.69E−49 | RBP11 | pSHF_mesoderm | 1.80E−195 |
| ATP6V1B2 | FHF_mesoderm | 6.27E−210 | SH3KBP1 | aSHF_mesoderm | 7.12E−47 | WNT8A | pSHF_mesoderm | 2.39E−184 |
| LZTS1 | FHF_mesoderm | 1.64E−209 | PCDH171 | aSHF_mesoderm | 8.48E−47 | HOXA1 | pSHF_mesoderm | 6.67E−183 |
| BMP2 | FHF_mesoderm | 3.22E−199 | RUNX1T1 | aSHF_mesoderm | 3.79E−45 | DCLK1 | pSHF_mesoderm | 2.89E−177 |
| DAAM1 | FHF_mesoderm | 1.70E−198 | ANPEP | aSHF_mesoderm | 7.04E−41 | IFITM3 | pSHF_mesoderm | 1.51E−165 |
| APOC1 | FHF_mesoderm | 8.16E−195 | PDGFRA | aSHF_mesoderm | 3.15E−40 | CITED2 | pSHF_mesoderm | 9.33E−165 |
| EOMES | FHF_mesoderm | 3.60E−189 | PGF | aSHF_mesoderm | 5.52E−38 | FOXH1 | pSHF_mesoderm | 2.64E−160 |
| HLX | FHF_mesoderm | 1.06E−173 | IGSF3 | aSHF_mesoderm | 8.01E−36 | FGF4 | pSHF_mesoderm | 2.52E−153 |
| OTX2 | FHF_mesoderm | 4.74E−160 | PAPPA | aSHF_mesoderm | 3.93E−33 | ALDH1A2 | pSHF_mesoderm | 1.08E−151 |
| UPP1 | FHF_mesoderm | 6.23E−156 | ADD3 | aSHF_mesoderm | 3.23E−32 | IFITM2 | pSHF_mesoderm | 1.86E−147 |
| FAM89A | FHF_mesoderm | 9.25E−149 | RCN1 | aSHF_mesoderm | 8.69E−30 | CTBP21 | pSHF_mesoderm | 1.24E−141 |
| APOA2 | FHF_mesoderm | 3.43E−148 | PITX1 | aSHF_mesoderm | 3.61E−29 | CCND2 | pSHF_mesoderm | 3.06E−137 |
| PDHA1 | FHF_mesoderm | 4.99E−139 | ITGA9 | aSHF_mesoderm | 6.36E−29 | CNTFR1 | pSHF_mesoderm | 1.88E−126 |
| PLCXD3 | FHF_mesoderm | 1.74E−128 | CHRNA3 | aSHF_mesoderm | 3.28E−27 | EFEMP1 | pSHF_mesoderm | 9.45E−113 |
| ABCD4 | FHF_mesoderm | 4.11E−126 | ANTXR2 | aSHF_mesoderm | 8.16E−26 | DLL11 | pSHF_mesoderm | 2.15E−104 |
| NPM3 | FHF_mesoderm | 2.52E−109 | IRX3 | aSHF_mesoderm | 1.69E−25 | DDX58 | pSHF_mesoderm | 4.68E−103 |
| AATF | FHF_mesoderm | 6.88E−100 | TMEM88 | aSHF_mesoderm | 2.89E−25 | MEST | pSHF_mesoderm | 1.02E−102 |
| KAZN | FHF_mesoderm | 1.11E−98 | CREG1 | aSHF_mesoderm | 1.90E−24 | ARL4C1 | pSHF_mesoderm | 1.87E−101 |
| VRTN | FHF_mesoderm | 5.98E−94 | FLNC | aSHF_mesoderm | 1.35E−23 | STMN1 | pSHF_mesoderm | 3.57E−101 |
| MYC | FHF_mesoderm | 2.26E−91 | TMEM51 | aSHF_mesoderm | 2.31E−23 | HMGB3 | pSHF_mesoderm | 5.96E−96 |
| PMEPA1 | FHF_mesoderm | 8.52E−82 | RND3 | aSHF_mesoderm | 3.25E−23 | GRM4 | pSHF_mesoderm | 5.81E−94 |
| CKB | FHF_mesoderm | 2.98E−79 | EFNB1 | aSHF_mesoderm | 5.63E−23 | SEPHS1 | pSHF_mesoderm | 3.68E−93 |
| YBX3 | FHF_mesoderm | 1.39E−70 | SQLE | aSHF_mesoderm | 2.30E−22 | LIMCH1 | pSHF_mesoderm | 1.01E−87 |
| LDB2 | FHF_mesoderm | 6.07E−70 | SOX9 | aSHF_mesoderm | 2.41E−22 | AP3B2 | pSHF_mesoderm | 1.90E−82 |
| ELMO1 | FHF_mesoderm | 1.86E−68 | CALU | aSHF_mesoderm | 3.86E−22 | PCLAF | pSHF_mesoderm | 3.59E−82 |
| TUBB4B | FHF_mesoderm | 3.21E−66 | RPP25 | aSHF_mesoderm | 2.71E−17 | LARP7 | pSHF_mesoderm | 3.43E−79 |
| VDAC2 | FHF_mesoderm | 2.12E−65 | KDELR1 | aSHF_mesoderm | 9.82E−16 | HMGA1 | pSHF_mesoderm | 1.10E−73 |
| TGFB1 | FHF_mesoderm | 1.60E−53 | CAP2 | aSHF_mesoderm | 2.22E−15 | SHROOM3 | pSHF_mesoderm | 4.32E−73 |
| GATA4 | FHF_mesoderm | 8.94E−52 | COTL1 | aSHF_mesoderm | 2.74E−15 | HAPLN3 | pSHF_mesoderm | 7.61E−71 |
| MIXL1 | FHF_mesoderm | 9.48E−52 | BRI3 | aSHF_mesoderm | 3.87E−15 | LEF11 | pSHF_mesoderm | 1.09E−68 |
| PIK3R1 | FHF_mesoderm | 1.63E−51 | NEO1 | aSHF_mesoderm | 4.00E−15 | TMEM59L | pSHF_mesoderm | 2.70E−68 |
| DIAPH2 | FHF_mesoderm | 8.48E−51 | BNIP3 | aSHF_mesoderm | 5.43E−15 | MTCL1 | pSHF_mesoderm | 1.71E−66 |
| RRBP1 | FHF_mesoderm | 1.24E−48 | ROBO1 | aSHF_mesoderm | 2.09E−13 | KIF5C1 | pSHF_mesoderm | 9.58E−64 |
| OST4 | FHF_mesoderm | 1.85E−45 | CLU | aSHF_mesoderm | 2.29E−13 | MFGE8 | pSHF_mesoderm | 2.46E−61 |
| ELL2 | FHF_mesoderm | 5.40E−43 | CASP6 | aSHF_mesoderm | 4.05E−10 | FN1 | pSHF_mesoderm | 3.35E−60 |
| PLEKHA6 | FHF_mesoderm | 6.23E−42 | JUN | aSHF_mesoderm | 4.09E−09 | SEMA6A | pSHF_mesoderm | 2.04E−57 |
| SLC35A3 | FHF_mesoderm | 2.21E−39 | SNX24 | aSHF_mesoderm | 7.89E−09 | TCF7 | pSHF_mesoderm | 2.78E−57 |
| PA2G4 | FHF_mesoderm | 2.09E−38 | SERINC3 | aSHF_mesoderm | 1.16E−08 | TPM1 | pSHF_mesoderm | 3.52E−57 |
| LIFR | FHF_mesoderm | 2.65E−38 | SERINC5 | aSHF_mesoderm | 1.51E−08 | PSAT1 | pSHF_mesoderm | 7.48E−57 |
| BCAT2 | FHF_mesoderm | 5.54E−37 | SLC16A1 | aSHF_mesoderm | 2.31E−08 | CENPW | pSHF_mesoderm | 9.96E−57 |
| BBX | FHF_mesoderm | 1.96E−34 | CNN3 | aSHF_mesoderm | 4.88E−07 | PHC2 | pSHF_mesoderm | 4.96E−55 |
| HAS2 | FHF_mesoderm | 1.38E−32 | PLIN3 | aSHF_mesoderm | 1.25E−06 | JAKMIP2 | pSHF_mesoderm | 1.29E−51 |
| STK26 | FHF_mesoderm | 1.65E−30 | ZNF462 | aSHF_mesoderm | 4.03E−06 | GALNT1 | pSHF_mesoderm | 8.67E−48 |
| CYCS | FHF_mesoderm | 1.34E−29 | SLC16A3 | aSHF_mesoderm | 1.77E−05 | CCDC160 | pSHF_mesoderm | 2.21E−47 |
| CHCHD10 | FHF_mesoderm | 1.32E−28 | ASPH | aSHF_mesoderm | 0.0003907 | ACTC1 | pSHF_mesoderm | 2.72E−47 |
| MDN1 | FHF_mesoderm | 4.82E−26 | CECR2 | aSHF_mesoderm | 0.0043529 | IFITM1 | pSHF_mesoderm | 5.68E−47 |
| EIF4A1 | FHF_mesoderm | 1.16E−25 | CTNNB1 | aSHF_mesoderm | 0.005793 | NSG1 | pSHF_mesoderm | 2.09E−44 |
| KIF16B | FHF_mesoderm | 1.47E−25 | | | | DPYSL21 | pSHF_mesoderm | 3.74E−44 |
| ZC3HAV1 | FHF_mesoderm | 1.62E−25 | | | | PIMREG | pSHF_mesoderm | 9.39E−44 |
| TRMT10C | FHF_mesoderm | 4.21E−25 | | | | STT3B | pSHF_mesoderm | 2.05E−42 |
| RUNX1 | FHF_mesoderm | 7.08E−25 | | | | FABP7 | pSHF_mesoderm | 1.84E−41 |
| APC | FHF_mesoderm | 6.36E−24 | | | | HOXB1 | pSHF_mesoderm | 3.27E−41 |
| LGR5 | FHF_mesoderm | 1.09E−23 | | | | FSTL1 | pSHF_mesoderm | 2.48E−39 |
| UQCRQ | FHF_mesoderm | 1.28E−23 | | | | MAGI1 | pSHF_mesoderm | 4.20E−39 |
| MT-ND5 | FHF_mesoderm | 9.15E−23 | | | | PTK7 | pSHF_mesoderm | 7.46E−39 |
| CXCL12 | FHF_mesoderm | 2.28E−21 | | | | ODC1 | pSHF_mesoderm | 9.00E−39 |
| ATF7IP2 | FHF_mesoderm | 3.02E−21 | | | | PBX1 | pSHF_mesoderm | 3.62E−38 |
| ZCCHC18 | FHF_mesoderm | 3.89E−19 | | | | EPHA1 | pSHF_mesoderm | 5.58E−38 |
| SOCS2 | FHF_mesoderm | 7.58E−19 | | | | GABBR1 | pSHF_mesoderm | 3.11E−37 |
| EIF3J | FHF_mesoderm | 1.29E−18 | | | | STXBP6 | pSHF_mesoderm | 3.20E−37 |
| ABHD4 | FHF_mesoderm | 1.87E−18 | | | | ITGB5 | pSHF_mesoderm | 1.11E−36 |
| DLC1 | FHF_mesoderm | 2.70E−18 | | | | ZNF423 | pSHF_mesoderm | 7.12E−36 |
| L1CAM | FHF_mesoderm | 1.74E−17 | | | | CHD71 | pSHF_mesoderm | 1.01E−35 |
| SPINT2 | FHF_mesoderm | 5.94E−17 | | | | CITED1 | pSHF_mesoderm | 2.56E−34 |
| TMCO1 | FHF_mesoderm | 2.76E−16 | | | | WIPF3 | pSHF_mesoderm | 2.71E−34 |
| JAKMIP1 | FHF_mesoderm | 2.88E−16 | | | | PCDH191 | pSHF_mesoderm | 4.03E−34 |
| TIPARP | FHF_mesoderm | 1.76E−15 | | | | PTPRK | pSHF_mesoderm | 6.10E−34 |
| ALDH2 | FHF_mesoderm | 5.30E−15 | | | | PHLDA2 | pSHF_mesoderm | 1.40E−33 |
| CHSY1 | FHF_mesoderm | 7.91E−15 | | | | HVCN11 | pSHF_mesoderm | 3.24E−33 |
| TRAPPC4 | FHF_mesoderm | 4.63E−14 | | | | TCEAL3 | pSHF_mesoderm | 3.63E−33 |
| TRIP10 | FHF_mesoderm | 2.91E−12 | | | | ALDH3A2 | pSHF_mesoderm | 4.48E−33 |
| SEC11C | FHF_mesoderm | 5.55E−12 | | | | MEIS21 | pSHF_mesoderm | 7.67E−33 |
| IMPDH2 | FHF_mesoderm | 5.72E−12 | | | | NAV2 | pSHF_mesoderm | 3.64E−32 |
| PAIP2 | FHF_mesoderm | 6.50E−11 | | | | TCEAL8 | pSHF_mesoderm | 1.04E−31 |
| AKAP13 | FHF_mesoderm | 6.68E−11 | | | | TSPAN18 | pSHF_mesoderm | 2.22E−31 |
| AGPAT2 | FHF_mesoderm | 1.55E−10 | | | | FGFR11 | pSHF_mesoderm | 1.08E−30 |
| ZNHIT3 | FHF_mesoderm | 6.48E−10 | | | | RAP2B | pSHF_mesoderm | 2.65E−30 |
| CCNDBP1 | FHF_mesoderm | 2.33E−09 | | | | C12orf75 | pSHF_mesoderm | 1.38E−29 |
| CERS4 | FHF_mesoderm | 2.39E−09 | | | | CRMP1 | pSHF_mesoderm | 1.93E−29 |
| GTPBP4 | FHF_mesoderm | 7.55E−09 | | | | GNAS | pSHF_mesoderm | 2.70E−29 |
| ABHD14A | FHF_mesoderm | 3.21E−07 | | | | HMGN5 | pSHF_mesoderm | 4.10E−29 |
| BRIX1 | FHF_mesoderm | 5.68E−07 | | | | MAPRE2 | pSHF_mesoderm | 4.43E−29 |
| CDC40 | FHF_mesoderm | 9.18E−07 | | | | RDH10 | pSHF_mesoderm | 4.48E−29 |
| GPATCH4 | FHF_mesoderm | 2.38E−06 | | | | IVNS1ABP | pSHF_mesoderm | 7.68E−29 |
| RNMT | FHF_mesoderm | 3.41E−06 | | | | AMOTL2 | pSHF_mesoderm | 8.52E−29 |
| RREB1 | FHF_mesoderm | 4.73E−06 | | | | NUDT11 | pSHF_mesoderm | 1.02E−28 |
| CBR1 | FHF_mesoderm | 7.38E−06 | | | | VASH2 | pSHF_mesoderm | 2.33E−28 |
| UCHL3 | FHF_mesoderm | 1.15E−05 | | | | YWHAQ | pSHF_mesoderm | 6.41E−28 |
| MYO10 | FHF_mesoderm | 1.90E−05 | | | | SDC1 | pSHF_mesoderm | 1.27E−27 |
| IFRD2 | FHF_mesoderm | 3.13E−05 | | | | MELK | pSHF_mesoderm | 1.33E−27 |
| ARID4B | FHF_mesoderm | 0.0004734 | | | | FHL1 | pSHF_mesoderm | 3.29E−26 |
| PDCD11 | FHF_mesoderm | 0.0006255 | | | | GORASP21 | pSHF_mesoderm | 4.10E−26 |
| | | | | | | FOXM1 | pSHF_mesoderm | 4.26E−26 |
| | | | | | | IGFBP4 | pSHF_mesoderm | 4.56E−26 |
| | | | | | | CRABP11 | pSHF_mesoderm | 7.76E−26 |
| | | | | | | IRAK1BP1 | pSHF_mesoderm | 1.65E−25 |
| | | | | | | PRMT6 | pSHF_mesoderm | 2.04E−24 |
| | | | | | | DDR1 | pSHF_mesoderm | 8.19E−24 |
| | | | | | | LHFPL6 | pSHF_mesoderm | 1.27E−23 |
| | | | | | | LRP1 | pSHF_mesoderm | 2.75E−23 |
| | | | | | | ING2 | pSHF_mesoderm | 5.46E−23 |
| | | | | | | RCN21 | pSHF_mesoderm | 6.15E−23 |
| | | | | | | GABARAPL | pSHF_mesoderm | 7.98E−23 |
| | | | | | | PSMC5 | pSHF_mesoderm | 3.03E−22 |
| | | | | | | PARP1 | pSHF_mesoderm | 3.15E−22 |
| | | | | | | DMKN1 | pSHF_mesoderm | 4.83E−22 |
| | | | | | | ZCRB11 | pSHF_mesoderm | 5.09E−22 |
| | | | | | | LSM6 | pSHF_mesoderm | 5.53E−22 |
| | | | | | | ETV4 | pSHF_mesoderm | 1.07E−21 |
| | | | | | | NOVA1 | pSHF_mesoderm | 1.44E−21 |
| | | | | | | POLA2 | pSHF_mesoderm | 3.61E−21 |
| | | | | | | LFNG | pSHF_mesoderm | 3.93E−21 |
| | | | | | | TCEAL5 | pSHF_mesoderm | 6.84E−21 |
| | | | | | | CRISPLD1 | pSHF_mesoderm | 1.08E−20 |
| | | | | | | USP18 | pSHF_mesoderm | 2.50E−20 |
| | | | | | | FGF31 | pSHF_mesoderm | 6.81E−20 |
| | | | | | | YWHAB | pSHF_mesoderm | 8.04E−20 |
| | | | | | | SPRED2 | pSHF_mesoderm | 8.73E−20 |
| | | | | | | TRMT11 | pSHF_mesoderm | 1.32E−19 |
| | | | | | | MMP21 | pSHF_mesoderm | 1.63E−19 |
| | | | | | | PDLIM5 | pSHF_mesoderm | 4.72E−19 |
| | | | | | | CHCHD7 | pSHF_mesoderm | 6.57E−19 |
| | | | | | | OAT | pSHF_mesoderm | 1.27E−18 |
| | | | | | | GINS4 | pSHF_mesoderm | 2.08E−18 |
| | | | | | | LIG1 | pSHF_mesoderm | 8.89E−18 |
| | | | | | | TACC21 | pSHF_mesoderm | 1.96E−17 |
| | | | | | | RAI14 | pSHF_mesoderm | 2.30E−17 |
| | | | | | | RGS10 | pSHF_mesoderm | 2.49E−17 |
| | | | | | | CASP3 | pSHF_mesoderm | 3.93E−17 |
| | | | | | | SS18L2 | pSHF_mesoderm | 7.43E−17 |
| | | | | | | FEN1 | pSHF_mesoderm | 9.27E−17 |
| | | | | | | MCM7 | pSHF_mesoderm | 1.16E−16 |
| | | | | | | UHRF1 | pSHF_mesoderm | 1.32E−16 |
| | | | | | | LHPP | pSHF_mesoderm | 1.56E−16 |
| | | | | | | KLF131 | pSHF_mesoderm | 1.77E−16 |
| | | | | | | METTL91 | pSHF_mesoderm | 1.97E−16 |
| | | | | | | SMARCD2 | pSHF_mesoderm | 3.47E−16 |
| | | | | | | HDGFL3 | pSHF_mesoderm | 3.60E−16 |
| | | | | | | NDRG3 | pSHF_mesoderm | 3.95E−16 |
| | | | | | | CLIC4 | pSHF_mesoderm | 7.97E−16 |
| | | | | | | DYNC1LI1 | pSHF_mesoderm | 8.54E−16 |
| | | | | | | PAFAH1B3 | pSHF_mesoderm | 1.04E−15 |
| | | | | | | ABCD3 | pSHF_mesoderm | 2.01E−15 |
| | | | | | | PREP | pSHF_mesoderm | 2.12E−15 |
| | | | | | | CHAF1B | pSHF_mesoderm | 2.64E−15 |
| | | | | | | FUBP11 | pSHF_mesoderm | 4.88E−15 |
| | | | | | | C8orf76 | pSHF_mesoderm | 5.03E−15 |
| | | | | | | GPX8 | pSHF_mesoderm | 7.57E−15 |
| | | | | | | SCAP | pSHF_mesoderm | 9.34E−15 |
| | | | | | | LPP | pSHF_mesoderm | 1.15E−14 |
| | | | | | | DIXDC1 | pSHF_mesoderm | 1.65E−14 |
| | | | | | | NSD2 | pSHF_mesoderm | 1.18E−13 |
| | | | | | | ADGRA3 | pSHF_mesoderm | 1.87E−13 |
| | | | | | | DUSP12 | pSHF_mesoderm | 2.15E−13 |
| | | | | | | PSMG4 | pSHF_mesoderm | 2.94E−13 |
| | | | | | | TM7SF3 | pSHF_mesoderm | 4.16E−13 |
| | | | | | | CAPN2 | pSHF_mesoderm | 8.21E−13 |
| | | | | | | ALDH18A1 | pSHF_mesoderm | 1.06E−12 |
| | | | | | | BCAT1 | pSHF_mesoderm | 1.27E−12 |
| | | | | | | FST1 | pSHF_mesoderm | 1.28E−12 |
| | | | | | | DPH6 | pSHF_mesoderm | 1.76E−12 |
| | | | | | | HEBP2 | pSHF_mesoderm | 3.05E−12 |
| | | | | | | MMD | pSHF_mesoderm | 3.41E−12 |
| | | | | | | PPP2R5E | pSHF_mesoderm | 4.07E−12 |
| | | | | | | KLHL42 | pSHF_mesoderm | 6.51E−12 |
| | | | | | | DLL31 | pSHF_mesoderm | 7.26E−12 |
| | | | | | | ARMCX2 | pSHF_mesoderm | 1.50E−11 |
| | | | | | | GPX3 | pSHF_mesoderm | 2.03E−11 |
| | | | | | | FOXN3 | pSHF_mesoderm | 2.44E−11 |
| | | | | | | DPP3 | pSHF_mesoderm | 2.55E−11 |
| | | | | | | TM9SF4 | pSHF_mesoderm | 2.56E−11 |
| | | | | | | SBK1 | pSHF_mesoderm | 4.09E−11 |
| | | | | | | AP3M2 | pSHF_mesoderm | 5.26E−11 |
| | | | | | | B3GAT3 | pSHF_mesoderm | 7.71E−11 |
| | | | | | | RECQL | pSHF_mesoderm | 8.36E−11 |
| | | | | | | SUV39H2 | pSHF_mesoderm | 9.04E−11 |
| | | | | | | FLRT3 | pSHF_mesoderm | 9.47E−11 |
| | | | | | | DCTPP1 | pSHF_mesoderm | 9.69E−11 |
| | | | | | | CDCA3 | pSHF_mesoderm | 1.34E−10 |
| | | | | | | HPF1 | pSHF_mesoderm | 1.39E−10 |
| | | | | | | RAB8B | pSHF_mesoderm | 2.89E−10 |
| | | | | | | ZDHHC6 | pSHF_mesoderm | 1.41E−09 |
| | | | | | | ARL2BP | pSHF_mesoderm | 2.36E−09 |
| | | | | | | COIL | pSHF_mesoderm | 2.61E−09 |
| | | | | | | C18orf21 | pSHF_mesoderm | 3.29E−09 |
| | | | | | | ZYX | pSHF_mesoderm | 3.67E−09 |
| | | | | | | AKR1B1 | pSHF_mesoderm | 4.31E−09 |
| | | | | | | SALL2 | pSHF_mesoderm | 6.19E−09 |
| | | | | | | CBX11 | pSHF_mesoderm | 6.25E−09 |
| | | | | | | ANXA11 | pSHF_mesoderm | 1.38E−08 |
| | | | | | | GOLPH3 | pSHF_mesoderm | 1.47E−08 |
| | | | | | | NDUFAF4 | pSHF_mesoderm | 1.48E−08 |
| | | | | | | RNF167 | pSHF_mesoderm | 3.00E−08 |
| | | | | | | FXN | pSHF_mesoderm | 3.02E−08 |
| | | | | | | RAB10 | pSHF_mesoderm | 3.78E−08 |
| | | | | | | RNF26 | pSHF_mesoderm | 7.49E−08 |
| | | | | | | TP53111 | pSHF_mesoderm | 1.06E−07 |
| | | | | | | MRPS61 | pSHF_mesoderm | 1.18E−07 |
| | | | | | | COL13A11 | pSHF_mesoderm | 1.69E−07 |
| | | | | | | SUMO3 | pSHF_mesoderm | 3.87E−07 |
| | | | | | | FBXO21 | pSHF_mesoderm | 6.06E−07 |
| | | | | | | UBE2D1 | pSHF_mesoderm | 7.16E−07 |
| | | | | | | PNP1 | pSHF_mesoderm | 7.54E−07 |
| | | | | | | B2M | pSHF_mesoderm | 8.16E−05 |
| | | | | | | BEX2 | pSHF_mesoderm | 0.000123 |
| | | | | | | SP51 | pSHF_mesoderm | 0.001251 |
| gene | cell type | p_val_adj | gene | cell type | p_val_adj | gene | cell type | p_val_adj |
| CCDC80 | FHF_progenitor | 0 | HAPLN1 | aSHF_progenitor | 0 | NR2F1 | pSHF_progenitor | 0 |
| TNNI1 | FHF_progenitor | 0 | JAG1 | aSHF_progenitor | 0 | NR2F2 | pSHF_progenitor | 0 |
| TNNT2 | FHF_progenitor | 0 | FN1 | aSHF_progenitor | 0 | HOXB1 | pSHF_progenitor | 0 |
| TNNC1 | FHF_progenitor | 0 | IRX3 | aSHF_progenitor | 0 | CPE | pSHF_progenitor | 0 |
| CSRP2 | FHF_progenitor | 0 | VCAN | aSHF_progenitor | 0 | TUBA1B | pSHF_progenitor | 0 |
| TPM1 | FHF_progenitor | 0 | MYOCD | aSHF_progenitor | 0 | DACH1 | pSHF_progenitor | 0 |
| KRT8 | FHF_progenitor | 1.69E−297 | RGS5 | aSHF_progenitor | 0 | TUBB | pSHF_progenitor | 0 |
| EMP2 | FHF_progenitor | 6.43E−296 | SALL1 | aSHF_progenitor | 0 | CDH2 | pSHF_progenitor | 0 |
| ACTC1 | FHF_progenitor | 9.08E−277 | TP53111 | aSHF_progenitor | 0 | MEIS3 | pSHF_progenitor | 0 |
| MYL41 | FHF_progenitor | 4.14E−271 | CDH11 | aSHF_progenitor | 0 | DUSP9 | pSHF_progenitor | 0 |
| CRIP2 | FHF_progenitor | 6.02E−248 | RPL36A | aSHF_progenitor | 0 | METRN | pSHF_progenitor | 0 |
| S100A10 | FHF_progenitor | 4.94E−222 | RPS21 | aSHF_progenitor | 0 | CYP26A1 | pSHF_progenitor | 0 |
| FRMD4B | FHF_progenitor | 4.08E−220 | LAPTM4B | aSHF_progenitor | 0 | RBMX | pSHF_progenitor | 0 |
| BAMBI1 | FHF_progenitor | 1.85E−202 | RPL38 | aSHF_progenitor | 0 | STMN1 | pSHF_progenitor | 0 |
| CALD11 | FHF_progenitor | 3.44E−201 | CITED2 | aSHF_progenitor | 0 | MARCKSL1 | pSHF_progenitor | 0 |
| VIM1 | FHF_progenitor | 1.22E−196 | MT-ND3 | aSHF_progenitor | 0 | PBX1 | pSHF_progenitor | 5.70E−277 |
| SLC1A3 | FHF_progenitor | 8.62E−150 | PDLIM1 | aSHF_progenitor | 0 | CRABP2 | pSHF_progenitor | 9.31E−262 |
| TMEM881 | FHF_progenitor | 2.50E−149 | FBN2 | aSHF_progenitor | 0 | VEGFC | pSHF_progenitor | 1.67E−245 |
| SIPA1L2 | FHF_progenitor | 2.31E−140 | RBM24 | aSHF_progenitor | 0 | ODC1 | pSHF_progenitor | 3.85E−234 |
| CRYAB | FHF_progenitor | 2.25E−126 | RPL37 | aSHF_progenitor | 0 | YWHAQ | pSHF_progenitor | 1.24E−194 |
| MAB21L2 | FHF_progenitor | 1.28E−123 | MEIS1 | aSHF_progenitor | 0 | HOXA2 | pSHF_progenitor | 1.27E−179 |
| SOX4 | FHF_progenitor | 4.60E−116 | TOMM7 | aSHF_progenitor | 0 | FZD7 | pSHF_progenitor | 3.76E−174 |
| UNC45B | FHF_progenitor | 5.23E−116 | CA2 | aSHF_progenitor | 0 | PPP1R14B | pSHF_progenitor | 4.40E−172 |
| CSRP3 | FHF_progenitor | 2.70E−114 | WNT5A | aSHF_progenitor | 0 | HOXA1 | pSHF_progenitor | 6.83E−171 |
| NPC2 | FHF_progenitor | 9.28E−108 | ELMO1 | aSHF_progenitor | 0 | PSIP1 | pSHF_progenitor | 2.37E−157 |
| NID21 | FHF_progenitor | 5.00E−107 | IRX5 | aSHF_progenitor | 0 | HOMER3 | pSHF_progenitor | 2.27E−152 |
| LBH1 | FHF_progenitor | 2.47E−106 | JARID2 | aSHF_progenitor | 0 | TLE1 | pSHF_progenitor | 5.01E−150 |
| HAND1 | FHF_progenitor | 1.22E−102 | SEC61G | aSHF_progenitor | 0 | MAD2L2 | pSHF_progenitor | 3.71E−149 |
| HCN4 | FHF_progenitor | 2.38E−101 | RPL37A | aSHF_progenitor | 0 | SKAP2 | pSHF_progenitor | 6.45E−139 |
| EZR1 | FHF_progenitor | 4.30E−98 | FGF10 | aSHF_progenitor | 0 | COX5A | pSHF_progenitor | 2.23E−134 |
| MYL61 | FHF_progenitor | 5.01E−94 | RPL34 | aSHF_progenitor | 0 | HMGB1 | pSHF_progenitor | 5.95E−108 |
| PPIC1 | FHF_progenitor | 4.23E−89 | IL17RD | aSHF_progenitor | 0 | UBE2E3 | pSHF_progenitor | 1.95E−101 |
| AHNAK1 | FHF_progenitor | 1.17E−88 | RPL27A | aSHF_progenitor | 0 | HMGN1 | pSHF_progenitor | 5.56E−95 |
| PRDM6 | FHF_progenitor | 1.39E−82 | RPS28 | aSHF_progenitor | 0 | NASP | pSHF_progenitor | 7.04E−95 |
| HSPB2 | FHF_progenitor | 3.07E−81 | RPL23 | aSHF_progenitor | 0 | CBX1 | pSHF_progenitor | 9.03E−91 |
| EIF4EBP1 | FHF_progenitor | 1.81E−80 | RPS15A | aSHF_progenitor | 0 | SLC25A5 | pSHF_progenitor | 9.29E−87 |
| SH3BGR | FHF_progenitor | 2.28E−80 | RPS23 | aSHF_progenitor | 0 | ANP32B | pSHF_progenitor | 7.57E−83 |
| TLN2 | FHF_progenitor | 1.17E−76 | RPS26 | aSHF_progenitor | 0 | SSRP1 | pSHF_progenitor | 1.35E−82 |
| MGARP | FHF_progenitor | 5.74E−76 | RPL36 | aSHF_progenitor | 0 | TOX3 | pSHF_progenitor | 2.80E−76 |
| COBLL1 | FHF_progenitor | 1.12E−75 | RPL22 | aSHF_progenitor | 0 | MYEF2 | pSHF_progenitor | 3.29E−71 |
| TBX5 | FHF_progenitor | 1.36E−75 | RPL30 | aSHF_progenitor | 0 | FUNDC2 | pSHF_progenitor | 1.49E−66 |
| VCAM1 | FHF_progenitor | 2.28E−75 | RPL35A | aSHF_progenitor | 0 | BAZ1A | pSHF_progenitor | 3.14E−62 |
| ACTA2 | FHF_progenitor | 1.32E−67 | RPS7 | aSHF_progenitor | 0 | ENY2 | pSHF_progenitor | 5.18E−57 |
| BVES | FHF_progenitor | 4.64E−67 | NPM3 | aSHF_progenitor | 2.49E−303 | NRIP1 | pSHF_progenitor | 1.20E−56 |
| BEX11 | FHF_progenitor | 5.20E−63 | ATP5F1E | aSHF_progenitor | 2.23E−300 | NAA38 | pSHF_progenitor | 1.84E−54 |
| ATP2B1 | FHF_progenitor | 5.44E−61 | PRDM1 | aSHF_progenitor | 1.89E−295 | NAP1L1 | pSHF_progenitor | 7.02E−54 |
| GPRC5C1 | FHF_progenitor | 5.72E−59 | APOE | aSHF_progenitor | 3.29E−261 | CYC1 | pSHF_progenitor | 1.51E−53 |
| POPDC3 | FHF_progenitor | 9.45E−59 | RPL22L1 | aSHF_progenitor | 5.79E−259 | PHF6 | pSHF_progenitor | 3.47E−49 |
| IDH21 | FHF_progenitor | 9.59E−58 | SH3KBP1 | aSHF_progenitor | 1.12E−257 | SMARCA5 | pSHF_progenitor | 3.02E−43 |
| FSCN11 | FHF_progenitor | 2.91E−57 | WLS | aSHF_progenitor | 1.62E−256 | TEAD2 | pSHF_progenitor | 2.30E−42 |
| TTN1 | FHF_progenitor | 3.42E−54 | CAP2 | aSHF_progenitor | 1.18E−253 | FOXF1 | pSHF_progenitor | 1.03E−41 |
| CDKN1C1 | FHF_progenitor | 7.22E−51 | RPS25 | aSHF_progenitor | 8.90E−253 | CCAR1 | pSHF_progenitor | 8.94E−41 |
| MAGED2 | FHF_progenitor | 4.47E−49 | RPS12 | aSHF_progenitor | 1.59E−248 | TSPAN13 | pSHF_progenitor | 3.26E−40 |
| SH3BGRL3 | FHF_progenitor | 4.18E−47 | IRX2 | aSHF_progenitor | 3.26E−247 | HNRNPAO | pSHF_progenitor | 1.42E−39 |
| CALM21 | FHF_progenitor | 8.23E−47 | RPS27A | aSHF_progenitor | 3.42E−247 | NDUFS8 | pSHF_progenitor | 5.27E−30 |
| PLS31 | FHF_progenitor | 5.16E−46 | RPL35 | aSHF_progenitor | 8.66E−243 | CDC20 | pSHF_progenitor | 5.64E−28 |
| TAGLN1 | FHF_progenitor | 1.42E−45 | NAV1 | aSHF_progenitor | 1.44E−235 | RANBP1 | pSHF_progenitor | 6.01E−25 |
| NEXN1 | FHF_progenitor | 4.86E−43 | SPRY2 | aSHF_progenitor | 1.10E−225 | SRRM2 | pSHF_progenitor | 3.96E−24 |
| POPDC2 | FHF_progenitor | 1.40E−42 | DLC1 | aSHF_progenitor | 3.46E−219 | RAB34 | pSHF_progenitor | 7.37E−24 |
| MDH21 | FHF_progenitor | 3.81E−42 | IFITM3 | aSHF_progenitor | 1.97E−218 | SNRPB | pSHF_progenitor | 2.39E−23 |
| PODXL | FHF_progenitor | 2.20E−40 | RPS13 | aSHF_progenitor | 3.77E−215 | PSMD7 | pSHF_progenitor | 7.88E−23 |
| FBXO32 | FHF_progenitor | 7.51E−39 | MID1 | aSHF_progenitor | 2.31E−212 | SRSF11 | pSHF_progenitor | 2.85E−22 |
| P4HA1 | FHF_progenitor | 2.16E−37 | LIFR | aSHF_progenitor | 2.72E−211 | THYN1 | pSHF_progenitor | 7.96E−22 |
| SPEG | FHF_progenitor | 1.38E−36 | DKK1.00 | aSHF_progenitor | 6.08E−202 | HEY1 | pSHF_progenitor | 5.73E−21 |
| LGALS1 | FHF_progenitor | 4.09E−36 | MEF2C | aSHF_progenitor | 2.96E−190 | WNT2 | pSHF_progenitor | 1.55E−19 |
| RHOC1 | FHF_progenitor | 1.20E−34 | NKX2-5 | aSHF_progenitor | 1.00E−173 | ACIN1 | pSHF_progenitor | 4.14E−19 |
| DPF3 | FHF_progenitor | 4.57E−32 | DAB2 | aSHF_progenitor | 6.14E−155 | NME1 | pSHF_progenitor | 8.92E−19 |
| CD81 | FHF_progenitor | 4.05E−31 | RPS17 | aSHF_progenitor | 3.58E−150 | CTCF | pSHF_progenitor | 1.84E−18 |
| ITM2A | FHF_progenitor | 6.41E−31 | RPL32 | aSHF_progenitor | 8.20E−147 | PRPF40A | pSHF_progenitor | 2.13E−18 |
| PCOLCE1 | FHF_progenitor | 1.73E−30 | FLNA | aSHF_progenitor | 2.07E−146 | RRM2 | pSHF_progenitor | 3.01E−17 |
| GLIPR21 | FHF_progenitor | 1.53E−28 | MT-ND2 | aSHF_progenitor | 2.50E−144 | RBM8A | pSHF_progenitor | 4.62E−17 |
| BMP21 | FHF_progenitor | 5.36E−27 | FGF8 | aSHF_progenitor | 1.71E−143 | MTF2 | pSHF_progenitor | 9.73E−17 |
| HOXB41 | FHF_progenitor | 2.88E−24 | UBL5 | aSHF_progenitor | 5.88E−123 | JAK1 | pSHF_progenitor | 9.88E−17 |
| TBX20 | FHF_progenitor | 6.56E−23 | RPL17 | aSHF_progenitor | 3.22E−110 | HOXB2 | pSHF_progenitor | 1.51E−16 |
| TMED9 | FHF_progenitor | 1.53E−21 | RPLP2 | aSHF_progenitor | 7.38E−104 | MTCH2 | pSHF_progenitor | 6.57E−16 |
| DNPEP | FHF_progenitor | 1.76E−21 | DUSP6 | aSHF_progenitor | 4.90E−101 | TBL1X | pSHF_progenitor | 1.58E−15 |
| GNB4 | FHF_progenitor | 1.41E−20 | MYL9 | aSHF_progenitor | 8.94E−101 | C1orf35 | pSHF_progenitor | 1.60E−15 |
| BCAM1 | FHF_progenitor | 1.87E−19 | RPL11 | aSHF_progenitor | 4.55E−100 | TMPO | pSHF_progenitor | 3.72E−14 |
| SMARCD3 | FHF_progenitor | 2.52E−19 | ROMO1 | aSHF_progenitor | 1.38E−98 | CEP57 | pSHF_progenitor | 1.54E−13 |
| MRPL12 | FHF_progenitor | 1.19E−18 | SALL4 | aSHF_progenitor | 1.02E−97 | HDAC2 | pSHF_progenitor | 2.85E−13 |
| ANKRD13 | FHF_progenitor | 1.69E−18 | EPHA4 | aSHF_progenitor | 1.13E−95 | ANP32E | pSHF_progenitor | 7.10E−13 |
| NRP11 | FHF_progenitor | 1.36E−17 | IRX1 | aSHF_progenitor | 9.57E−87 | MCM7 | pSHF_progenitor | 7.72E−13 |
| FDPS1 | FHF_progenitor | 1.74E−17 | HAND2 | aSHF_progenitor | 2.22E−83 | SNN | pSHF_progenitor | 2.34E−12 |
| BAG3 | FHF_progenitor | 1.43E−16 | MSI2 | aSHF_progenitor | 5.77E−83 | ZNF706 | pSHF_progenitor | 8.80E−12 |
| ATP1A11 | FHF_progenitor | 1.93E−16 | ACSL3 | aSHF_progenitor | 8.76E−83 | SPSB4 | pSHF_progenitor | 5.54E−11 |
| ATP5F1B1 | FHF_progenitor | 2.58E−16 | SPRY1 | aSHF_progenitor | 6.12E−76 | NHP2 | pSHF_progenitor | 3.81E−10 |
| TRAPPC4 | FHF_progenitor | 1.67E−15 | SPRED1 | aSHF_progenitor | 1.54E−74 | CFL2 | pSHF_progenitor | 2.82E−09 |
| PKP21 | FHF_progenitor | 1.85E−15 | PRTG | aSHF_progenitor | 1.17E−73 | RBCK1 | pSHF_progenitor | 5.73E−08 |
| CCND2 | FHF_progenitor | 3.91E−15 | MGST1 | aSHF_progenitor | 4.38E−72 | KIF20A | pSHF_progenitor | 1.22E−07 |
| CREB3L1 | FHF_progenitor | 1.43E−14 | ISL1 | aSHF_progenitor | 1.24E−71 | TRIM27 | pSHF_progenitor | 1.24E−07 |
| CGNL11 | FHF_progenitor | 1.75E−13 | PDLIM7 | aSHF_progenitor | 5.03E−68 | NSMCE2 | pSHF_progenitor | 5.15E−07 |
| AKR1A11 | FHF_progenitor | 1.97E−13 | BMP7 | aSHF_progenitor | 1.50E−67 | PABPC4 | pSHF_progenitor | 1.94E−06 |
| CIAPIN11 | FHF_progenitor | 2.01E−13 | COX7B | aSHF_progenitor | 1.95E−64 | SHISA2 | pSHF_progenitor | 1.17E−05 |
| ACAA21 | FHF_progenitor | 2.54E−13 | B3GALNT1 | aSHF_progenitor | 6.29E−64 | E2F5 | pSHF_progenitor | 1.36E−05 |
| COX171 | FHF_progenitor | 5.53E−13 | SIX1 | aSHF_progenitor | 1.67E−60 | C1orf43 | pSHF_progenitor | 1.39E−05 |
| CMTM71 | FHF_progenitor | 6.09E−13 | FSTL1 | aSHF_progenitor | 2.82E−59 | HSPH1 | pSHF_progenitor | 1.84E−05 |
| PTH1R1 | FHF_progenitor | 7.65E−13 | LOXL2 | aSHF_progenitor | 1.14E−58 | VEZF1 | pSHF_progenitor | 1.98E−05 |
| EMILIN21 | FHF_progenitor | 7.70E−13 | PAM | aSHF_progenitor | 3.72E−58 | | | |
| PPIB | FHF_progenitor | 1.04E−12 | RCSD1 | aSHF_progenitor | 8.81E−54 | | | |
| PIM31 | FHF_progenitor | 1.34E−12 | CTDSPL2 | aSHF_progenitor | 6.02E−53 | | | |
| LRRFIP11 | FHF_progenitor | 7.95E−12 | MPPED2 | aSHF_progenitor | 7.07E−53 | | | |
| EIF2B3 | FHF_progenitor | 1.35E−11 | PFN1 | aSHF_progenitor | 1.36E−52 | | | |
| ANXA61 | FHF_progenitor | 5.51E−11 | TPM2 | aSHF_progenitor | 1.44E−50 | | | |
| UBC1 | FHF_progenitor | 7.13E−11 | DNMT3B | aSHF_progenitor | 1.58E−50 | | | |
| TBCB1 | FHF_progenitor | 2.25E−10 | OGT | aSHF_progenitor | 4.78E−47 | | | |
| CALU | FHF_progenitor | 3.07E−10 | FLNC | aSHF_progenitor | 4.22E−44 | | | |
| BTG3 | FHF_progenitor | 3.64E−10 | GNG11 | aSHF_progenitor | 4.52E−43 | | | |
| ATP2A21 | FHF_progenitor | 3.93E−10 | YWHAZ | aSHF_progenitor | 3.58E−40 | | | |
| ADK | FHF_progenitor | 6.76E−10 | ATP5ME | aSHF_progenitor | 8.80E−40 | | | |
| PRR5 | FHF_progenitor | 7.48E−10 | EML4 | aSHF_progenitor | 2.78E−38 | | | |
| SPARC1 | FHF_progenitor | 9.88E−10 | MT-CO1 | aSHF_progenitor | 6.52E−38 | | | |
| KLF13 | FHF_progenitor | 1.13E−09 | ACTN1 | aSHF_progenitor | 1.30E−36 | | | |
| RNH1 | FHF_progenitor | 2.02E−09 | RHOU | aSHF_progenitor | 7.72E−30 | | | |
| MSX1 | FHF_progenitor | 2.31E−09 | WDR76 | aSHF_progenitor | 3.02E−25 | | | |
| ARPC2 | FHF_progenitor | 2.58E−09 | BNIP3 | aSHF_progenitor | 5.04E−22 | | | |
| PDIA3 | FHF_progenitor | 2.90E−09 | SMS | aSHF_progenitor | 1.55E−21 | | | |
| ID3 | FHF_progenitor | 4.12E−09 | TGFB1 | aSHF_progenitor | 4.89E−18 | | | |
| EIF2S21 | FHF_progenitor | 7.14E−09 | DHX9 | aSHF_progenitor | 2.11E−17 | | | |
| APBB1 | FHF_progenitor | 8.63E−09 | HIGD1A | aSHF_progenitor | 6.81E−17 | | | |
| ERCC11 | FHF_progenitor | 1.24E−08 | VASP | aSHF_progenitor | 3.00E−15 | | | |
| IRF2BPL | FHF_progenitor | 2.55E−08 | PTBP1 | aSHF_progenitor | 1.51E−10 | | | |
| SH3GLB11 | FHF_progenitor | 6.26E−08 | IFITM2 | aSHF_progenitor | 4.28E−09 | | | |
| CST31 | FHF_progenitor | 7.40E−08 | | | | | | |
| CYCS1 | FHF_progenitor | 8.23E−08 | | | | | | |
| SNRPN1 | FHF_progenitor | 1.92E−07 | | | | | | |
| P4HB | FHF_progenitor | 2.00E−07 | | | | | | |
| POLR2G1 | FHF_progenitor | 4.95E−07 | | | | | | |
| ATP6VOB | FHF_progenitor | 5.51E−07 | | | | | | |
| UBA3 | FHF_progenitor | 1.16E−06 | | | | | | |
| ARF1 | FHF_progenitor | 1.80E−06 | | | | | | |
| YIF1B | FHF_progenitor | 5.18E−06 | | | | | | |
| DAD1 | FHF_progenitor | 5.73E−06 | | | | | | |
| ATP1B1 | FHF_progenitor | 9.49E−06 | | | | | | |
| DYNC1LI1 | FHF_progenitor | 2.21E−05 | | | | | | |
| PSMD61 | FHF_progenitor | 2.57E−05 | | | | | | |
| TSPAN4 | FHF_progenitor | 2.69E−05 | | | | | | |
| MRPS18B | FHF_progenitor | 7.02E−05 | | | | | | |
| RBPMS2 | FHF_progenitor | 7.47E−05 | | | | | | |
| BMP41 | FHF_progenitor | 9.87E−05 | | | | | | |
| TUBB4B1 | FHF_progenitor | 0.0001183 | | | | | | |
| RHEB | FHF_progenitor | 0.0001978 | | | | | | |
| EFNB3 | FHF_progenitor | 0.0014556 | | | | | | |
| AK3 | FHF_progenitor | 0.0038194 | | | | | | |
| HMMR1 | FHF_progenitor | 0.0361776 | | | | | | |

| gene | cell type | p_val_adj | gene | cell type | p_val_adj |
|---|---|---|---|---|---|
| BMP2 | AVCCM | 2.61E−110 | RPL29 | LVCM | 5.68E−131 |
| ATP1B1 | AVCCM | 4.01E−96 | RPL12 | LVCM | 3.02E−129 |
| CPNE5 | AVCCM | 5.80E−85 | RPL13 | LVCM | 3.34E−125 |
| ATP1A1 | AVCCM | 1.20E−73 | RPS14 | LVCM | 1.32E−110 |
| TNNT2 | AVCCM | 1.76E−59 | RPL18 | LVCM | 1.35E−105 |
| BAMBI | AVCCM | 1.40E−48 | RPL18A | LVCM | 5.08E−97 |
| STMN1 | AVCCM | 2.31E−43 | RPS8 | LVCM | 1.48E−96 |
| NDUFA4 | AVCCM | 3.25E−42 | RPL32 | LVCM | 2.30E−94 |
| TMSB4X | AVCCM | 3.97E−40 | RPS3 | LVCM | 8.40E−94 |
| BTG1 | AVCCM | 5.54E−40 | RPS7 | LVCM | 9.00E−94 |
| FHL2 | AVCCM | 6.71E−40 | RPL15 | LVCM | 7.05E−91 |
| CRIP2 | AVCCM | 4.29E−38 | RPL11 | LVCM | 2.10E−90 |
| LTBP1 | AVCCM | 1.41E−37 | RPL3 | LVCM | 3.83E−90 |
| CHCHD10 | AVCCM | 1.06E−36 | RPL5 | LVCM | 7.28E−89 |
| RSPO3 | AVCCM | 6.28E−35 | RPL8 | LVCM | 5.00E−86 |
| SERF2 | AVCCM | 6.77E−35 | RPS12 | LVCM | 1.39E−84 |
| ASAH1 | AVCCM | 2.42E−33 | RPS6 | LVCM | 6.84E−84 |
| ATP5F1E | AVCCM | 3.68E−32 | RPL19 | LVCM | 1.63E−82 |
| TBX2 | AVCCM | 6.05E−32 | RPS5 | LVCM | 6.46E−82 |
| PRDX2 | AVCCM | 3.53E−30 | RPS23 | LVCM | 8.33E−81 |
| ATP5PF | AVCCM | 1.35E−27 | RPL7 | LVCM | 4.06E−78 |
| FAM78A | AVCCM | 1.91E−26 | RPS19 | LVCM | 3.75E−76 |
| COX6B1 | AVCCM | 1.03E−25 | EEF1B2 | LVCM | 3.57E−70 |
| DSTN | AVCCM | 1.57E−25 | RPL6 | LVCM | 6.22E−70 |
| MTCH1 | AVCCM | 7.34E−25 | HEY2 | LVCM | 3.65E−69 |
| CACNA1D | AVCCM | 1.15E−24 | HSP90AB1 | LVCM | 3.12E−66 |
| MYL9 | AVCCM | 2.56E−21 | NACA | LVCM | 3.23E−65 |
| MAGED2 | AVCCM | 6.53E−21 | RPL30 | LVCM | 4.51E−65 |
| ID4 | AVCCM | 1.45E−20 | RPL13A | LVCM | 1.07E−64 |
| TBX3 | AVCCM | 2.28E−20 | RPS9 | LVCM | 7.77E−64 |
| COX6C | AVCCM | 1.75E−18 | NPM1 | LVCM | 1.11E−59 |
| COX7A1 | AVCCM | 4.11E−18 | RPS16 | LVCM | 3.18E−56 |
| GOLIM4 | AVCCM | 6.37E−18 | RPS15A | LVCM | 1.45E−49 |
| FBXO32 | AVCCM | 9.34E−18 | RPL24 | LVCM | 1.57E−48 |
| BEX4 | AVCCM | 1.33E−17 | RPL35A | LVCM | 1.60E−48 |
| LMOD2 | AVCCM | 1.52E−17 | RPS27A | LVCM | 1.62E−45 |
| ITM2C | AVCCM | 8.81E−17 | RPL28 | LVCM | 5.19E−42 |
| NDUFA11 | AVCCM | 1.55E−16 | BTF3 | LVCM | 6.29E−42 |
| ATP1B3 | AVCCM | 1.68E−16 | RPSA | LVCM | 2.58E−39 |
| B2M | AVCCM | 1.94E−16 | NAV1 | LVCM | 1.59E−38 |
| NKX2-5 | AVCCM | 2.58E−16 | RPS15 | LVCM | 3.78E−37 |
| MGST3 | AVCCM | 6.72E−16 | FABP5 | LVCM | 6.61E−37 |
| MAP1LC3A | AVCCM | 7.02E−16 | RPL37 | LVCM | 2.70E−33 |
| TRDN | AVCCM | 1.47E−15 | RPL35 | LVCM | 5.22E−33 |
| WNT2 | AVCCM | 3.06E−15 | RPL37A | LVCM | 5.58E−33 |
| CFL1 | AVCCM | 4.19E−15 | RPS11 | LVCM | 7.63E−33 |
| TLN1 | AVCCM | 5.52E−15 | RPL22 | LVCM | 1.70E−30 |
| NDUFA1 | AVCCM | 7.81E−15 | RPL34 | LVCM | 1.65E−29 |
| UBL5 | AVCCM | 1.40E−14 | HMGA1 | LVCM | 9.10E−29 |
| MRPS6 | AVCCM | 3.18E−14 | RPL21 | LVCM | 2.36E−28 |
| NDUFA13 | AVCCM | 1.08E−13 | MTUS2 | LVCM | 2.26E−27 |
| TNNI3 | AVCCM | 1.35E−13 | RPS13 | LVCM | 6.45E−27 |
| MORF4L1 | AVCCM | 7.57E−13 | RPL23 | LVCM | 2.39E−26 |
| GSN | AVCCM | 2.59E−12 | FITM1 | LVCM | 2.43E−26 |
| ATP5PO | AVCCM | 3.57E−12 | RPL27A | LVCM | 2.17E−23 |
| TMEM50A | AVCCM | 3.88E−12 | RPL36 | LVCM | 1.78E−22 |
| TRIM8 | AVCCM | 4.38E−12 | ARHGAP29 | LVCM | 3.25E−22 |
| NDUFC1 | AVCCM | 1.10E−11 | SERBP1 | LVCM | 3.74E−22 |
| SLC25A4 | AVCCM | 2.87E−11 | IRX4 | LVCM | 1.42E−21 |
| TUBA1A | AVCCM | 3.01E−11 | HAND1 | LVCM | 8.81E−21 |
| MYL4 | AVCCM | 3.73E−11 | EFNB3 | LVCM | 3.24E−20 |
| FBLIM1 | AVCCM | 4.52E−11 | XIRP1 | LVCM | 4.38E−20 |
| ETFB | AVCCM | 4.72E−11 | RBM3 | LVCM | 2.04E−18 |
| MYOZ1 | AVCCM | 5.90E−11 | TOMM20 | LVCM | 6.99E−18 |
| ATP5IF1 | AVCCM | 9.03E−11 | LAMA4 | LVCM | 2.05E−17 |
| HMOX2 | AVCCM | 1.05E−10 | ANP32B | LVCM | 4.71E−17 |
| NDUFA6 | AVCCM | 1.28E−10 | EIF3M | LVCM | 5.38E−17 |
| ARMCX3 | AVCCM | 1.33E−10 | RSL1D1 | LVCM | 9.98E−17 |
| TPGS1 | AVCCM | 1.41E−10 | IMPDH2 | LVCM | 1.02E−15 |
| PDLIM7 | AVCCM | 2.34E−10 | NAP1L1 | LVCM | 1.39E−15 |
| RTN4 | AVCCM | 3.22E−10 | FBXL22 | LVCM | 1.72E−15 |
| MPC2 | AVCCM | 3.31E−10 | EIF3D | LVCM | 2.66E−15 |
| FBN2 | AVCCM | 1.12E−09 | EIF4EBP1 | LVCM | 5.15E−15 |
| ITM2B | AVCCM | 1.45E−09 | RPS27 | LVCM | 5.84E−15 |
| CAMTA1 | AVCCM | 1.69E−09 | PCDH7 | LVCM | 1.20E−14 |
| NDRG2 | AVCCM | 2.94E−09 | APEX1 | LVCM | 1.73E−14 |
| EID1 | AVCCM | 3.58E−09 | TLN2 | LVCM | 2.98E−14 |
| NDUFB3 | AVCCM | 3.64E−09 | RPS25 | LVCM | 1.53E−12 |
| CAPNS1 | AVCCM | 4.16E−09 | ANGPT1 | LVCN | 1.69E−12 |
| FERMT2 | AVCCM | 5.39E−09 | RPLP2 | LVCM | 3.08E−12 |
| ANXA6 | AVCCM | 6.58E−09 | SMYD2 | LVCM | 3.50E−12 |
| GABARAPL2 | AVCCM | 6.66E−09 | EIF3A | LVCM | 1.10E−11 |
| MSX2 | AVCCM | 8.92E−09 | TYMS | LVCM | 2.99E−10 |
| IGFBP5 | AVCCM | 9.43E−09 | POLR1D | LVCM | 4.32E−10 |
| RCAN1 | AVCCM | 9.90E−09 | AHCY | LVCM | 5.55E−10 |
| PBXIP1 | AVCCM | 2.22E−08 | CCND2 | LVCM | 8.06E−10 |
| DBN1 | AVCCM | 4.50E−08 | PHGDH | LVCM | 1.38E−09 |
| CLIC1 | AVCCM | 5.68E−08 | EIF4A1 | LVCM | 1.69E−09 |
| UBB | AVCCM | 9.70E−08 | IGFBP2 | LVCM | 6.01E−09 |
| ACAA2 | AVCCM | 1.14E−07 | HSPD1 | LVCM | 8.68E−09 |
| PDLIM5 | AVCCM | 1.16E−07 | NOB1 | LVCM | 3.10E−08 |
| MICAL2 | AVCCM | 1.17E−07 | SNRPB | LVCM | 4.50E−08 |
| ATP5ME | AVCCM | 2.08E−07 | NRP1 | LVCM | 6.56E−08 |
| SPARC | AVCCM | 2.31E−07 | CCT7 | LVCM | 2.50E−07 |
| TCEAL8 | AVCCM | 2.58E−07 | TPM4 | LVCM | 8.28E−07 |
| HOXB2 | AVCCM | 2.78E−07 | PABPC4 | LVCM | 1.58E−06 |
| SLC39A8 | AVCCM | 2.96E−07 | RAN | LVCM | 2.18E−06 |
| CCDC141 | AVCCM | 3.54E−07 | TAF1D | LVCM | 2.42E−06 |
| ATP5PD | AVCCM | 4.01E−07 | CDV3 | LVCM | 5.13E−06 |
| PTP4A2 | AVCCM | 8.65E−07 | MYH7 | LVCM | 7.24E−06 |
| CAPZA2 | AVCCM | 1.03E−06 | MIF | LVCM | 8.65E−06 |
| DYNLRB1 | AVCCM | 1.93E−06 | CCT4 | LVCM | 8.88E−06 |
| NFE2L2 | AVCCM | 2.34E−06 | CCT2 | LVCM | 2.97E−05 |
| CCDC28B | AVCCM | 2.41E−06 | SMS | LVCM | 5.25E−05 |
| MAST4 | AVCCM | 2.57E−06 | TAGLN | LVCM | 0.00015247 |
| RABAC1 | AVCCM | 2.75E−06 | C1QBP | LVCM | 0.00025991 |
| S100A13 | AVCCM | 3.00E−06 | RBMX | LVCM | 0.00028054 |
| ECH1 | AVCCM | 3.02E−06 | MTHFD2 | LVCM | 0.00029777 |
| RAB14 | AVCCM | 3.20E−06 | BOP1 | LVCM | 0.00034669 |
| HK1 | AVCCM | 3.42E−06 | EIF3B | LVCM | 0.00042155 |
| SQLE | AVCCM | 3.52E−06 | NOP58 | LVCM | 0.00051688 |
| PPP1R12A | AVCCM | 3.77E−06 | TKT | LVCM | 0.00063685 |
| DPYSL3 | AVCCM | 6.37E−06 | CCT3 | LVCM | 0.00069585 |
| GLRX | AVCCM | 8.77E−06 | GADD45G | LVCM | 0.00082898 |
| VDAC3 | AVCCM | 9.13E−06 | LRPPRC | LVCM | 0.00134791 |
| ITGB1 | AVCCM | 1.01E−05 | FBL | LVCM | 0.00308971 |
| ATP5MF | AVCCM | 1.07E−05 | WNT5A | LVCM | 0.00323285 |
| SLC25A11 | AVCCM | 1.14E−05 | OLA1 | LVCM | 0.00369838 |
| TBX5 | AVCCM | 1.31E−05 | CHKA | LVCM | 0.00505275 |
| BEX2 | AVCCM | 1.32E−05 | EIF2S3 | LVCM | 0.00511693 |
| COMMD6 | AVCCM | 1.48E−05 | CKS2 | LVCM | 0.00528231 |
| ATP6VOB | AVCCM | 1.71E−05 | SMCHD1 | LVCM | 0.00729422 |
| THRA | AVCCM | 1.85E−05 | C4orf3 | LVCM | 0.01194317 |
| CALM2 | AVCCM | 2.09E−05 | SUCLG2 | LVCM | 0.01242707 |
| PEA15 | AVCCM | 2.28E−05 | C12orf45 | LVCM | 0.01417031 |
| PGAM2 | AVCCM | 6.49E−05 | CYBA | LVCM | 0.01502716 |
| PPP1R3A | AVCCM | 7.80E−05 | ST13 | LVCM | 0.01912399 |
| TMA7 | AVCCM | 7.96E−05 | SNRPD1 | LVCM | 0.0231748 |
| MYH6 | AVCCM | 8.88E−05 | DDX21 | LVCM | 0.04961646 |
| HADHA | AVCCM | 0.000104523 | | | |
| HIPK2 | AVCCM | 0.000111496 | | | |
| BEX1 | AVCCM | 0.000174876 | | | |
| PPARGC1A | AVCCM | 0.000184801 | | | |
| DUSP6 | AVCCM | 0.000255731 | | | |
| TRAPPC1 | AVCCM | 0.000336336 | | | |
| DACT1 | AVCCM | 0.000349512 | | | |
| ARHGAP1 | AVCCM | 0.000701187 | | | |
| TGFB2 | AVCCM | 0.00079094 | | | |
| DRAP1 | AVCCM | 0.001023401 | | | |
| YWHAB | AVCCM | 0.001186618 | | | |
| TCEA3 | AVCCM | 0.001234083 | | | |
| HSD17B12 | AVCCM | 0.001361998 | | | |
| REEP5 | AVCCM | 0.001551709 | | | |
| ATP6AP2 | AVCCM | 0.001711567 | | | |
| TUBB2A | AVCCM | 0.002135619 | | | |
| EPHA7 | AVCCM | 0.002310982 | | | |
| ASPH | AVCCM | 0.002557134 | | | |
| TXLNB | AVCCM | 0.005313325 | | | |
| VAMP2 | AVCCM | 0.007595862 | | | |
| ARL3 | AVCCM | 0.008788277 | | | |
| NDN | AVCCM | 0.009218734 | | | |
| ATOX1 | AVCCM | 0.010241494 | | | |
| TBCB | AVCCM | 0.010435354 | | | |
| TSPAN13 | AVCCM | 0.01077701 | | | |
| NCOR1 | AVCCM | 0.012296565 | | | |
| SGCB | AVCCM | 0.014526151 | | | |
| TMBIM6 | AVCCM | 0.014723328 | | | |
| RBFOX2 | AVCCM | 0.01477042 | | | |
| PJA2 | AVCCM | 0.014843082 | | | |
| LAMTOR2 | AVCCM | 0.018881237 | | | |
| TCTA | AVCCM | 0.021976205 | | | |
| APOE | AVCCM | 0.026199094 | | | |
| BTBD1 | AVCCM | 0.026347112 | | | |
| RAP2C | AVCCM | 0.032323025 | | | |
| ANAPC13 | AVCCM | 0.0329497 | | | |
| AKR1A1 | AVCCM | 0.034387841 | | | |
| CTSB | AVCCM | 0.036808459 | | | |
| FDPS | AVCCM | 0.036990836 | | | |
| GINM1 | AVCCM | 0.037327226 | | | |
| UBE2L6 | AVCCM | 0.040574651 | | | |
| TMEM59 | AVCCM | 0.044081957 | | | |
| SYPL1 | AVCCM | 0.045149844 | | | |

| gene | cell type | p_val_adj | gene | cell type | p_val_adj |
|---|---|---|---|---|---|
| CSRP2 | OFTCM | 1.04E−166 | PGK1 | RVCM | 4.62E−154 |
| PEG10 | OFTCM | 2.13E−90 | LDHA | RVCM | 3.52E−147 |
| LAPTM4A | OFTCM | 7.96E−82 | GAPDH | RVCM | 1.78E−133 |
| PLCXD3 | OFTCM | 4.70E−70 | TPI1 | RVCM | 6.98E−122 |
| MTUS2 | OFTCM | 3.55E−65 | CSRP3 | RVCM | 5.56E−120 |
| ANK3 | OFTCM | 1.60E−64 | PLN | RVCM | 6.23E−119 |
| MYL6 | OFTCM | 4.57E−63 | EPHA4 | RVCM | 9.30E−115 |
| VEGFC | OFTCM | 2.36E−61 | ATP1B1 | RVCM | 4.36E−107 |
| HAND2 | OFTCM | 1.84E−56 | GPI | RVCM | 1.02E−101 |
| VCAN | OFTCM | 6.57E−54 | FHL2 | RVCM | 2.00E−99 |
| LAPTM4B | OFTCM | 2.63E−52 | C1QTNF4 | RVCM | 4.14E−96 |
| CFC1 | OFTCM | 1.14E−47 | MYL2 | RVCM | 2.55E−91 |
| DUSP1 | OFTCM | 2.37E−44 | MIF | RVCM | 8.25E−91 |
| IFITM3 | OFTCM | 2.69E−43 | LMOD2 | RVCM | 2.73E−78 |
| SEMA3C | OFTCM | 1.85E−39 | CRIP2 | RVCM | 2.80E−77 |
| TGFB111 | OFTCM | 2.22E−38 | TRDN | RVCM | 3.12E−73 |
| ID1 | OFTCM | 5.09E−37 | IRX1 | RVCM | 4.21E−72 |
| PCDH7 | OFTCM | 7.63E−35 | COX6A2 | RVCM | 4.35E−69 |
| SSR3 | OFTCM | 2.87E−34 | LRRC10 | RVCM | 7.41E−65 |
| TXN | OFTCM | 6.53E−33 | TNNI3 | RVCM | 1.59E−62 |
| CSRP1 | OFTCM | 1.63E−32 | CHCHD10 | RVCM | 2.69E−61 |
| TPM2 | OFTCM | 1.17E−30 | TCAP | RVCM | 1.60E−59 |
| NFIL3 | OFTCM | 4.66E−29 | NPPB | RVCM | 1.71E−57 |
| SGCE | OFTCM | 8.22E−26 | PFKP | RVCM | 1.02E−55 |
| MEIS2 | OFTCM | 8.51E−25 | HSPB8 | RVCM | 7.52E−55 |
| SRSF3 | OFTCM | 2.11E−24 | NPPA | RVCM | 1.43E−51 |
| CPE | OFTCM | 7.12E−24 | ASPH | RVCM | 5.36E−50 |
| ID3 | OFTCM | 6.86E−23 | IRX2 | RVCM | 1.22E−49 |
| RHOA | OFTCM | 2.28E−21 | ADPRHL1 | RVCM | 2.83E−41 |
| OST4 | OFTCM | 2.56E−21 | PDK1 | RVCM | 3.65E−41 |
| PRNP | OFTCM | 3.91E−21 | TFRC | RVCM | 1.43E−39 |
| HMGN1 | OFTCM | 1.84E−20 | DSP | RVCM | 7.27E−38 |
| IFITM2 | OFTCM | 3.62E−20 | MYL3 | RVCM | 8.58E−37 |
| OCIAD2 | OFTCM | 5.84E−20 | MT-ND6 | RVCM | 2.30E−35 |
| GNG12 | OFTCM | 4.23E−19 | VDAC1 | RVCM | 3.79E−35 |
| PIM1 | OFTCM | 1.70E−18 | ALCAM | RVCM | 2.14E−34 |
| IGSF3 | OFTCM | 1.81E−18 | DKK3.00 | RVCM | 4.90E−32 |
| TGIF1 | OFTCM | 9.21E−18 | MYH6 | RVCM | 5.48E−31 |
| CNN2 | OFTCM | 2.45E−17 | FLNC | RVCM | 2.90E−30 |
| PFN1 | OFTCM | 1.16E−16 | LRRC39 | RVCM | 3.82E−30 |
| FZD1 | OFTCM | 3.61E−16 | VDAC2 | RVCM | 1.44E−29 |
| TIPARP | OFTCM | 5.54E−16 | ALDOA | RVCM | 3.71E−29 |
| TAGLN | OFTCM | 7.40E−16 | HK1 | RVCM | 4.69E−29 |
| CDK6 | OFTCM | 1.37E−15 | MYL4 | RVCM | 1.88E−27 |
| NRP2 | OFTCM | 1.61E−15 | VEGFB | RVCM | 1.31E−25 |
| DOK4 | OFTCM | 2.32E−15 | COX5B | RVCM | 2.44E−24 |
| NUAK1 | OFTCM | 2.79E−15 | NDUFA4 | RVCM | 4.39E−23 |
| B3GALNT1 | OFTCM | 4.55E−15 | PDE4DIP | RVCM | 2.25E−22 |
| TMEM65 | OFTCM | 4.98E−15 | MYBPC3 | RVCM | 7.66E−21 |
| ARL2BP | OFTCM | 5.86E−15 | PDLIM5 | RVCM | 2.48E−20 |
| DYNLL1 | OFTCM | 8.31E−15 | ATP5PO | RVCM | 3.88E−19 |
| OSTC | OFTCM | 1.17E−14 | BSG | RVCM | 6.22E−19 |
| SRSF7 | OFTCM | 2.39E−14 | SYNPO2L | RVCM | 6.41E−19 |
| WNT5A | OFTCM | 1.44E−13 | MYH7 | RVCM | 3.24E−18 |
| SFPQ | OFTCM | 2.59E−13 | ATP5PF | RVCM | 1.14E−16 |
| TENM3 | OFTCM | 3.11E−13 | DUSP10 | RVCM | 3.95E−16 |
| HAND1 | OFTCM | 4.67E−13 | GAMT | RVCM | 3.66E−15 |
| RCN1 | OFTCM | 5.64E−13 | NDUFB4 | RVCM | 4.39E−15 |
| MPHOSPH6 | OFTCM | 6.73E−13 | TTN | RVCM | 1.61E−14 |
| ACTA2 | OFTCM | 1.02E−12 | NDUFA8 | RVCM | 7.30E−14 |
| KLF6 | OFTCM | 2.29E−12 | MYOZ1 | RVCM | 7.71E−14 |
| KLF3 | OFTCM | 1.51E−11 | ECI1 | RVCM | 8.72E−14 |
| PARD6B | OFTCM | 3.17E−11 | MYOZ2 | RVCM | 2.96E−13 |
| MFAP4 | OFTCM | 6.57E−11 | TRIM54 | RVCM | 3.03E−13 |
| SDCBP | OFTCM | 7.50E−11 | ACTN2 | RVCM | 4.39E−12 |
| CKAP4 | OFTCM | 1.05E−10 | SOD2 | RVCM | 6.19E−12 |
| CALR | OFTCM | 1.66E−10 | COX7B | RVCM | 6.51E−12 |
| CALD1 | OFTCM | 1.94E−10 | SLC39A8 | RVCM | 6.99E−12 |
| SRSF2 | OFTCM | 2.98E−10 | NDUFA6 | RVCM | 1.10E−11 |
| LMOD1 | OFTCM | 7.61E−10 | CPNE5 | RVCM | 1.21E−11 |
| TBX20 | OFTCM | 7.87E−10 | COX7A2 | RVCM | 1.57E−11 |
| PLS3 | OFTCM | 1.59E−09 | COX8A | RVCM | 6.27E−11 |
| CDH3 | OFTCM | 2.60E−09 | MLIP | RVCM | 6.47E−11 |
| UBE2E2 | OFTCM | 3.67E−09 | MGST3 | RVCM | 2.75E−10 |
| ATP2B1 | OFTCM | 4.17E−09 | LBH | RVCM | 4.42E−10 |
| PARVA | OFTCM | 8.76E−09 | TNNC1 | RVCM | 5.22E−10 |
| PFN2 | OFTCM | 1.34E−08 | ATP5F1B | RVCM | 5.93E−10 |
| CFL1 | OFTCM | 1.76E−08 | MYZAP | RVCM | 1.70E−09 |
| DERL1 | OFTCM | 2.03E−08 | TCEA3 | RVCM | 1.94E−09 |
| TMEM237 | OFTCM | 2.48E−08 | FABP3 | RVCM | 6.51E−09 |
| BLMH | OFTCM | 2.93E−08 | CYCS | RVCM | 8.31E−09 |
| PPIB | OFTCM | 7.36E−08 | COX6C | RVCM | 4.21E−08 |
| COL1A1 | OFTCM | 7.45E−08 | SNTA1 | RVCM | 1.23E−07 |
| RBP1 | OFTCM | 7.51E−08 | MRPL41 | RVCM | 1.31E−07 |
| HMGB3 | OFTCM | 7.72E−08 | ATP2A2 | RVCM | 2.15E−07 |
| DDOST | OFTCM | 2.41E−07 | CISD1 | RVCM | 2.89E−07 |
| SOX4 | OFTCM | 2.49E−07 | TMEM38A | RVCM | 7.55E−07 |
| PAWR | OFTCM | 2.73E−07 | SERPINE2 | RVCM | 1.08E−06 |
| ACTB | OFTCM | 2.94E−07 | PPP1R12B | RVCM | 2.05E−06 |
| FHL1 | OFTCM | 5.80E−07 | WVNK1 | RVCM | 4.70E−06 |
| LPP | OFTCM | 7.91E−07 | SLC8A1 | RVCM | 4.75E−06 |
| RSU1 | OFTCM | 8.39E−07 | OBSCN | RVCM | 4.76E−06 |
| ARF4 | OFTCM | 1.18E−06 | ACO2 | RVCM | 4.78E−06 |
| HMBOX1 | OFTCM | 2.03E−06 | ZBTB20 | RVCM | 4.79E−06 |
| ASAP1 | OFTCM | 2.21E−06 | COX6A1 | RVCM | 2.24E−05 |
| NPNT | OFTCM | 2.46E−06 | CRYAB | RVCM | 3.38E−05 |
| CNN3 | OFTCM | 2.49E−06 | IDH3A | RVCM | 0.00013972 |
| GATA6 | OFTCM | 3.16E−06 | TXLNB | RVCM | 0.00015406 |
| ZC3H15 | OFTCM | 3.43E−06 | ATP5PD | RVCM | 0.00015519 |
| CDH11 | OFTCM | 3.77E−06 | MPC2 | RVCM | 0.00023456 |
| WAC | OFTCM | 4.25E−06 | USP13 | RVCM | 0.00061919 |
| CEP290 | OFTCM | 5.13E−06 | GOT1 | RVCM | 0.00462426 |
| SRSF10 | OFTCM | 7.04E−06 | SDHA | RVCM | 0.00472314 |
| CCT2 | OFTCM | 1.39E−05 | FUNDC2 | RVCM | 0.00668935 |
| SGMS1 | OFTCM | 1.56E−05 | HOMER1 | RVCM | 0.01013301 |
| LIMD2 | OFTCM | 1.64E−05 | TRIB2 | RVCM | 0.01176992 |
| SNX6 | OFTCM | 1.98E−05 | MYO18B | RVCM | 0.05092774 |
| HNRNPM | OFTCM | 3.15E−05 | | | |
| EML4 | OFTCM | 3.65E−05 | | | |
| PACSIN3 | OFTCM | 4.66E−05 | | | |
| KRT8 | OFTCM | 6.52E−05 | | | |
| SH3GLB1 | OFTCM | 9.21E−05 | | | |
| ACTN1 | OFTCM | 9.67E−05 | | | |
| TP53111 | OFTCM | 0.00010784 | | | |
| GLYR1 | OFTCM | 0.00011513 | | | |
| ACTG1 | OFTCM | 0.00011687 | | | |
| RBM25 | OFTCM | 0.00013318 | | | |
| CLINT1 | OFTCM | 0.00017126 | | | |
| IMPACT | OFTCM | 0.00019227 | | | |
| FRG1 | OFTCM | 0.00022095 | | | |
| IGF2BP3 | OFTCM | 0.00024547 | | | |
| FAM177A1 | OFTCM | 0.00024596 | | | |
| PSMD14 | OFTCM | 0.00024704 | | | |
| GADD45G | OFTCM | 0.00029438 | | | |
| CCT3 | OFTCM | 0.0004271 | | | |
| COPZ1 | OFTCM | 0.00045236 | | | |
| GTF3C6 | OFTCM | 0.00048091 | | | |
| TRA2A | OFTCM | 0.00077822 | | | |
| BAZ2B | OFTCM | 0.00095342 | | | |
| SUMO3 | OFTCM | 0.000969 | | | |
| KCTD10 | OFTCM | 0.00126661 | | | |
| RBBP4 | OFTCM | 0.00169212 | | | |
| TRMT112 | OFTCM | 0.00250653 | | | |
| LMO4 | OFTCM | 0.00337401 | | | |
| ACTN4 | OFTCM | 0.0038488 | | | |
| PUF60 | OFTCM | 0.00398678 | | | |
| DNAJB11 | OFTCM | 0.00421731 | | | |
| MLLT11 | OFTCM | 0.00476554 | | | |
| TPM4 | OFTCM | 0.00537186 | | | |
| KRT18 | OFTCM | 0.00619594 | | | |
| CTBP1 | OFTCM | 0.00701469 | | | |
| BCLAF1 | OFTCM | 0.00762463 | | | |
| SNX2 | OFTCM | 0.00829119 | | | |
| SERP1 | OFTCM | 0.00928003 | | | |
| HNRNPD | OFTCM | 0.00972623 | | | |
| FRMD4B | OFTCM | 0.00981603 | | | |
| PCGF5 | OFTCM | 0.01196855 | | | |
| LIMS1 | OFTCM | 0.01227844 | | | |
| MEF2C | OFTCM | 0.01420594 | | | |
| GCC2 | OFTCM | 0.01507451 | | | |
| SEC13 | OFTCM | 0.04376187 | | | |
| SMYD1 | OFTCM | 0.09180249 | | | |
| ARPC5 | OFTCM | 0.15146355 | | | |
| PGK1 | OFTCM | 4.62E−154 | | | |
| LDHA | OFTCM | 3.52E−147 | | | |
| GAPDH | OFTCM | 1.78E−133 | | | |
| TPI1 | OFTCM | 6.98E−122 | | | |
| CSRP3 | OFTCM | 5.56E−120 | | | |
| PLN | OFTCM | 6.23E−119 | | | |
| EPHA4 | OFTCM | 9.30E−115 | | | |
| ATP1B1 | OFTCM | 4.36E−107 | | | |
| GPI | OFTCM | 1.02E−101 | | | |
| FHL2 | OFTCM | 2.00E−99 | | | |
| C1QTNF4 | OFTCM | 4.14E−96 | | | |
| MYL2 | OFTCM | 2.55E−91 | | | |
| MIF | OFTCM | 8.25E−91 | | | |
| LMOD2 | OFTCM | 2.73E−78 | | | |
| CRIP2 | OFTCM | 2.80E−77 | | | |
| TRDN | OFTCM | 3.12E−73 | | | |
| IRX1 | OFTCM | 4.21E−72 | | | |
| COX6A2 | OFTCM | 4.35E−69 | | | |
| LRRC10 | OFTCM | 7.41E−65 | | | |
| LRRC39 | OFTCM | 3.82E−30 | | | |
| VDAC2 | OFTCM | 1.44E−29 | | | |
| ALDOA | OFTCM | 3.71E−29 | | | |
| HK1 | OFTCM | 4.69E−29 | | | |
| MYL4 | OFTCM | 1.88E−27 | | | |
| VEGFB | OFTCM | 1.31E−25 | | | |
| COX5B | OFTCM | 2.44E−24 | | | |
| NDUFA4 | OFTCM | 4.39E−23 | | | |
| PDE4DIP | OFTCM | 2.25E−22 | | | |
| MYBPC3 | OFTCM | 7.66E−21 | | | |
| PDLIM5 | OFTCM | 2.48E−20 | | | |
| ATP5PO | OFTCM | 3.88E−19 | | | |
| BSG | OFTCM | 6.22E−19 | | | |
| SYNPO2L | OFTCM | 6.41E−19 | | | |
| MYH7 | OFTCM | 3.24E−18 | | | |
| ATP5PF | OFTCM | 1.14E−16 | | | |
| DUSP10 | OFTCM | 3.95E−16 | | | |
| GAMT | OFTCM | 3.66E−15 | | | |
| NDUFB4 | OFTCM | 4.39E−15 | | | |
| TTN | OFTCM | 1.61E−14 | | | |
| NDUFA8 | OFTCM | 7.30E−14 | | | |
| MYOZ1 | OFTCM | 7.71E−14 | | | |
| ECI1 | OFTCM | 8.72E−14 | | | |
| MYOZ2 | OFTCM | 2.96E−13 | | | |
| TRIM54 | OFTCM | 3.03E−13 | | | |
| ACTN2 | OFTCM | 4.39E−12 | | | |
| SOD2 | OFTCM | 6.19E−12 | | | |
| COX7B | OFTCM | 6.51E−12 | | | |
| SLC39A8 | OFTCM | 6.99E−12 | | | |
| NDUFA6 | OFTCM | 1.10E−11 | | | |
| CPNE5 | OFTCM | 1.21E−11 | | | |
| COX7A2 | OFTCM | 1.57E−11 | | | |
| COX8A | OFTCM | 6.27E−11 | | | |
| MLIP | OFTCM | 6.47E−11 | | | |
| MGST3 | OFTCM | 2.75E−10 | | | |
| LBH | OFTCM | 4.42E−10 | | | |
| TNNC1 | OFTCM | 5.22E−10 | | | |
| ATP5F1B | OFTCM | 5.93E−10 | | | |
| MYZAP | OFTCM | 1.70E−09 | | | |
| TCEA3 | OFTCM | 1.94E−09 | | | |
| FABP3 | OFTCM | 6.51E−09 | | | |
| CYCS | OFTCM | 8.31E−09 | | | |
| COX6C | OFTCM | 4.21E−08 | | | |
| SNTA1 | OFTCM | 1.23E−07 | | | |
| MRPL41 | OFTCM | 1.31E−07 | | | |
| ATP2A2 | OFTCM | 2.15E−07 | | | |
| CISD1 | OFTCM | 2.89E−07 | | | |
| TMEM38A | OFTCM | 7.55E−07 | | | |
| SERPINE2 | OFTCM | 1.08E−06 | | | |
| PPP1R12B | OFTCM | 2.05E−06 | | | |
| WNK1 | OFTCM | 4.70E−06 | | | |
| SLC8A1 | OFTCM | 4.75E−06 | | | |
| OBSCN | OFTCM | 4.76E−06 | | | |
| ACO2 | OFTCM | 4.78E−06 | | | |
| ZBTB20 | OFTCM | 4.79E−06 | | | |
| COX6A1 | OFTCM | 2.24E−05 | | | |
| CRYAB | OFTCM | 3.38E−05 | | | |
| IDH3A | OFTCM | 0.00013972 | | | |
| TXLNB | OFTCM | 0.00015406 | | | |
| ATP5PD | OFTCM | 0.00015519 | | | |
| MPC2 | OFTCM | 0.00023456 | | | |
| USP13 | OFTCM | 0.00061919 | | | |
| GOT1 | OFTCM | 0.00462426 | | | |
| SDHA | OFTCM | 0.00472314 | | | |
| FUNDC2 | OFTCM | 0.00668935 | | | |
| HOMER1 | OFTCM | 0.01013301 | | | |
| TRIB2 | OFTCM | 0.01176992 | | | |
| MYO18B | OFTCM | 0.05092774 | | | |

| gene | cell type | p_val_adj | gene | cell type | p_val_adj |
|---|---|---|---|---|---|
| COL1A2 | SVCM | 1.14E−210 | MYH6 | ACM | 3.17E−115 |
| TMSB4X | SVCM | 1.06E−120 | TNNC1 | ACM | 5.37E−115 |
| FLNA | SVCM | 4.29E−106 | MYL3 | ACM | 8.80E−108 |
| PLAT | SVCM | 4.17E−103 | TTN | ACM | 3.22E−93 |
| SPARC | SVCM | 3.34E−102 | TNNT2 | ACM | 1.33E−88 |
| ACTB | SVCM | 6.16E−97 | COX6A2 | ACM | 1.40E−88 |
| S100A10 | SVCM | 2.83E−96 | MYBPC3 | ACM | 5.20E−79 |
| CALD1 | SVCM | 3.51E−92 | ENO3 | ACM | 1.03E−78 |
| IFITM3 | SVCM | 7.10E−91 | ACTN2 | ACM | 1.59E−78 |
| CRABP2 | SVCM | 9.33E−89 | NPPA | ACM | 1.65E−68 |
| ACTG1 | SVCM | 8.78E−88 | FABP3 | ACM | 1.43E−66 |
| CNN3 | SVCM | 1.31E−85 | CRYAB | ACM | 1.65E−66 |
| C4orf48 | SVCM | 2.29E−85 | PGAM2 | ACM | 6.00E−66 |
| FN1 | SVCM | 1.33E−84 | MYL7 | ACM | 1.13E−65 |
| MARCKS | SVCM | 4.82E−80 | BMP7 | ACM | 2.07E−65 |
| LIMA1 | SVCM | 1.54E−76 | CSRP3 | ACM | 4.40E−64 |
| SH3BGRL3 | SVCM | 1.29E−67 | SLC25A4 | ACM | 3.08E−62 |
| TUBA1A | SVCM | 1.57E−67 | TCAP | ACM | 1.55E−60 |
| TMSB10 | SVCM | 4.03E−67 | CRIP2 | ACM | 3.21E−60 |
| MARCKSL1 | SVCM | 3.20E−66 | ATP2A2 | ACM | 6.68E−60 |
| FLRT2 | SVCM | 8.92E−65 | LDB3 | ACM | 1.77E−54 |
| MFAP4 | SVCM | 3.30E−64 | SMPX | ACM | 4.25E−52 |
| VIM | SVCM | 2.39E−63 | CITED4 | ACM | 1.54E−47 |
| SFRP1 | SVCM | 8.29E−57 | LMOD2 | ACM | 1.55E−41 |
| TGFB2 | SVCM | 4.72E−56 | NDUFA4 | ACM | 3.94E−39 |
| HMGN1 | SVCM | 5.19E−55 | TCEA3 | ACM | 4.24E−39 |
| CADM1 | SVCM | 8.55E−55 | COX5A | ACM | 3.92E−37 |
| COTL1 | SVCM | 4.78E−52 | TMOD1 | ACM | 1.70E−36 |
| TPM4 | SVCM | 2.42E−49 | MYLK3 | ACM | 3.34E−36 |
| SLC9A3R1 | SVCM | 1.48E−46 | MYL4 | ACM | 4.16E−36 |
| S100A11 | SVCM | 4.87E−42 | PAM | ACM | 2.62E−34 |
| ATP2B1 | SVCM | 4.85E−41 | RYR2 | ACM | 9.39E−32 |
| FBLN1 | SVCM | 6.84E−41 | TNNI3 | ACM | 1.24E−31 |
| GNAS | SVCM | 7.28E−41 | NKX2-5 | ACM | 2.11E−31 |
| SESN3 | SVCM | 1.33E−39 | ATP1B1 | ACM | 4.57E−30 |
| PTN | SVCM | 1.38E−39 | QKI | ACM | 9.77E−28 |
| CXCL12 | SVCM | 3.21E−38 | TRIM54 | ACM | 3.40E−27 |
| PPP1R14A | SVCM | 1.26E−37 | EIF1B | ACM | 5.26E−27 |
| TAGLN2 | SVCM | 1.76E−37 | FSD2 | ACM | 5.32E−27 |
| IGFBP4 | SVCM | 1.24E−35 | HSPB2 | ACM | 4.38E−26 |
| PRDX6 | SVCM | 4.38E−35 | ATP5F1D | ACM | 5.88E−26 |
| LBH | SVCM | 1.50E−33 | GOT1 | ACM | 1.06E−25 |
| SFRP5 | SVCM | 3.43E−33 | COX8A | ACM | 6.20E−25 |
| RRBP1 | SVCM | 3.74E−33 | HSPB7 | ACM | 7.18E−24 |
| MEST | SVCM | 7.43E−33 | NDUFA11 | ACM | 9.28E−24 |
| FTL | SVCM | 1.02E−32 | SYNPO2L | ACM | 1.21E−23 |
| RBP1 | SVCM | 4.82E−32 | SLCO3A1 | ACM | 1.84E−23 |
| CD63 | SVCM | 5.94E−31 | ATP5F1C | ACM | 3.14E−23 |
| FOXP2 | SVCM | 9.26E−31 | NDUFB10 | ACM | 3.58E−23 |
| CFL1 | SVCM | 1.47E−30 | GAPDH | ACM | 2.71E−22 |
| GPC3 | SVCM | 1.98E−29 | APOBEC2 | ACM | 5.37E−22 |
| TENM3 | SVCM | 4.59E−28 | KLHL31 | ACM | 1.49E−21 |
| IFITM2 | SVCM | 7.51E−28 | VDAC2 | ACM | 8.98E−21 |
| SOX4 | SVCM | 1.19E−27 | CAV1 | ACM | 1.11E−20 |
| PPIB | SVCM | 2.55E−27 | RBM38 | ACM | 5.06E−20 |
| NME4 | SVCM | 2.65E−27 | VDAC1 | ACM | 1.71E−19 |
| TPM3 | SVCM | 2.82E−27 | LMO7 | ACM | 3.47E−19 |
| SRSF9 | SVCM | 1.35E−26 | KCNJ5 | ACM | 8.36E−19 |
| RCN2 | SVCM | 1.64E−26 | MYO18B | ACM | 9.23E−19 |
| IQGAP1 | SVCM | 1.24E−25 | MT-ND4 | ACM | 1.62E−18 |
| TBX18 | SVCM | 1.33E−25 | FITM1 | ACM | 2.06E−18 |
| BEX1 | SVCM | 1.25E−24 | MYBPHL | ACM | 6.42E−18 |
| FABP5 | SVCM | 2.41E−24 | TRIM55 | ACM | 9.69E−18 |
| DLK1 | SVCM | 3.31E−24 | SLC25A3 | ACM | 1.98E−17 |
| CTHRC1 | SVCM | 4.28E−24 | HSPB3 | ACM | 3.71E−17 |
| TMOD3 | SVCM | 7.16E−24 | TMEM163 | ACM | 7.45E−17 |
| PGRMC1 | SVCM | 8.53E−23 | SH3KBP1 | ACM | 3.76E−16 |
| SH3BGRL | SVCM | 1.09E−22 | ITGB1BP2 | ACM | 2.12E−15 |
| SEMA3C | SVCM | 5.99E−22 | IDH2 | ACM | 3.88E−15 |
| SEC61B | SVCM | 6.15E−22 | COX411 | ACM | 6.37E−15 |
| DDX5 | SVCM | 8.54E−22 | COX5B | ACM | 1.50E−14 |
| LYAR | SVCM | 3.26E−21 | PFKP | ACM | 1.73E−14 |
| CALR | SVCM | 4.65E−21 | SORBS2 | ACM | 1.92E−14 |
| PBX3 | SVCM | 7.34E−21 | SLC25A5 | ACM | 4.41E−14 |
| PDIA3 | SVCM | 5.68E−20 | BNIP3 | ACM | 5.88E−14 |
| MAGED2 | SVCM | 1.56E−19 | HADHB | ACM | 4.97E−13 |
| DPYSL3 | SVCM | 1.75E−19 | DSP | ACM | 5.91E−13 |
| CTXN1 | SVCM | 4.14E−19 | HINT1 | ACM | 2.08E−12 |
| SEC11A | SVCM | 1.56E−18 | POPDC2 | ACM | 2.65E−12 |
| BZW1 | SVCM | 7.14E−18 | NDUFA6 | ACM | 4.97E−12 |
| WFDC2 | SVCM | 7.48E−18 | GAMT | ACM | 8.28E−12 |
| CYFIP1 | SVCM | 3.68E−17 | MT-CYB | ACM | 2.12E−11 |
| NPM1 | SVCM | 6.97E−17 | DKK3.00 | ACM | 2.34E−11 |
| MFAP2 | SVCM | 8.50E−17 | NDUFV3 | ACM | 2.47E−11 |
| MYH10 | SVCM | 1.36E−16 | COX7C | ACM | 2.79E−11 |
| LITAF | SVCM | 1.75E−16 | FHOD3 | ACM | 5.20E−11 |
| CORO1C | SVCM | 1.78E−16 | RRAD | ACM | 7.45E−11 |
| ZFP36L1 | SVCM | 4.05E−16 | CYCS | ACM | 7.86E−11 |
| TENM4 | SVCM | 5.01E−16 | ITGA6 | ACM | 1.12E−10 |
| PLK2 | SVCM | 5.75E−16 | ATP5F1B | ACM | 1.23E−10 |
| TUBB | SVCM | 2.18E−15 | GALK1 | ACM | 1.99E−10 |
| PPP1R14B | SVCM | 2.73E−15 | UQCR10 | ACM | 3.45E−10 |
| RHOC | SVCM | 3.62E−15 | CORO6 | ACM | 6.32E−10 |
| ECE1 | SVCM | 4.03E−15 | GHITM | ACM | 1.17E−09 |
| F2R | SVCM | 4.29E−15 | COX7B | ACM | 1.25E−09 |
| STMN1 | SVCM | 4.58E−15 | ATP5PO | ACM | 1.28E−09 |
| RSPO3 | SVCM | 5.42E−15 | COX6C | ACM | 1.39E−09 |
| RAB5C | SVCM | 3.52E−14 | ATP5PF | ACM | 1.64E−09 |
| TCF4 | SVCM | 5.12E−14 | CHCHD10 | ACM | 2.91E−09 |
| RPL12 | SVCM | 5.33E−14 | FBXL22 | ACM | 4.08E−09 |
| HNRNPAO | SVCM | 6.61E−14 | VDAC3 | ACM | 5.33E−09 |
| CMTM3 | SVCM | 7.22E−14 | RBPMS2 | ACM | 8.13E−09 |
| DUSP5 | SVCM | 7.91E−14 | USP13 | ACM | 1.73E−08 |
| AKAP5 | SVCM | 8.97E−14 | PDK1 | ACM | 1.87E−08 |
| TOP1 | SVCM | 9.77E−14 | UQCRFS1 | ACM | 2.30E−08 |
| RPS9 | SVCM | 9.78E−14 | SDHA | ACM | 4.15E−08 |
| PDCD4 | SVCM | 1.32E−13 | ATP5F1A | ACM | 4.46E−08 |
| RCN1 | SVCM | 1.46E−13 | NDUFA5 | ACM | 4.77E−08 |
| RPL3 | SVCM | 1.69E−13 | PTGES3L | ACM | 4.93E−08 |
| SMOC2 | SVCM | 2.05E−13 | ATP5PD | ACM | 9.95E−08 |
| TGIF1 | SVCM | 3.25E−13 | PPP1R14C | ACM | 1.50E−07 |
| TUBA1B | SVCM | 9.57E−13 | BCAM | ACM | 2.05E−07 |
| BIN1 | SVCM | 9.81E−13 | PTP4A3 | ACM | 2.31E−07 |
| HOMER3 | SVCM | 2.22E−12 | COX6B1 | ACM | 2.46E−07 |
| ADAMTS1 | SVCM | 4.76E−12 | TPM1 | ACM | 2.66E−07 |
| MGARP | SVCM | 5.07E−12 | ATP1A1 | ACM | 4.61E−07 |
| GADD45G | SVCM | 7.27E−12 | FLNC | ACM | 6.26E−07 |
| HSP90B1 | SVCM | 3.32E−11 | ACO2 | ACM | 6.65E−07 |
| RPS6 | SVCM | 5.06E−11 | ANKRD1 | ACM | 9.58E−07 |
| ARL6IP1 | SVCM | 5.93E−11 | MDH1 | ACM | 1.11E−06 |
| RPS7 | SVCM | 6.15E−11 | NDUFA8 | ACM | 1.74E−06 |
| ARF4 | SVCM | 6.67E−11 | LPGAT1 | ACM | 1.94E−06 |
| RANBP1 | SVCM | 7.14E−11 | NDRG2 | ACM | 4.11E−06 |
| PNRC2 | SVCM | 1.03E−10 | LRRC39 | ACM | 8.05E−06 |
| EIF4G2 | SVCM | 1.81E−10 | SMARCD3 | ACM | 9.43E−06 |
| NOS1AP | SVCM | 6.65E−10 | EMP2 | ACM | 1.40E−05 |
| CNTFR | SVCM | 9.75E−10 | IDH3A | ACM | 1.48E−05 |
| DDOST | SVCM | 1.22E−09 | ALPK3 | ACM | 1.57E−05 |
| UNC5C | SVCM | 2.38E−09 | OBSCN | ACM | 2.68E−05 |
| SLIT3 | SVCM | 3.66E−09 | SOD2 | ACM | 2.84E−05 |
| FNDC3A | SVCM | 1.67E−08 | NDUFB11 | ACM | 3.77E−05 |
| HSPA8 | SVCM | 1.73E−08 | CPNE5 | ACM | 4.07E−05 |
| GNAI3 | SVCM | 1.89E−08 | PGK1 | ACM | 4.32E−05 |
| PLAGL1 | SVCM | 2.27E−08 | HRC | ACM | 8.28E−05 |
| FUS | SVCM | 2.35E−08 | HADHA | ACM | 9.19E−05 |
| PEG10 | SVCM | 4.11E−08 | FAM162A | ACM | 0.000119208 |
| AKR1A1 | SVCM | 5.77E−08 | MDH2 | ACM | 0.000130433 |
| TMEM98 | SVCM | 6.90E−08 | ATP5MC3 | ACM | 0.000138457 |
| RPS12 | SVCM | 1.53E−07 | ASPH | ACM | 0.000201845 |
| CALM2 | SVCM | 1.77E−07 | ATP5ME | ACM | 0.000262567 |
| CLTA | SVCM | 1.83E−07 | SUCLG1 | ACM | 0.000364436 |
| RPL6 | SVCM | 2.72E−07 | FBLIM1 | ACM | 0.000519504 |
| ENY2 | SVCM | 2.97E−07 | GPI | ACM | 0.000666012 |
| MXD4 | SVCM | 3.19E−07 | NDUFS6 | ACM | 0.000730636 |
| RPL28 | SVCM | 3.20E−07 | NDUFS7 | ACM | 0.001497415 |
| RPL36 | SVCM | 3.24E−07 | XIRP1 | ACM | 0.001606398 |
| NEDD4 | SVCM | 3.27E−07 | UNC45B | ACM | 0.001917287 |
| JAM3 | SVCM | 3.77E−07 | RASSF5 | ACM | 0.001955651 |
| PDIA6 | SVCM | 7.39E−07 | BVES | ACM | 0.002639301 |
| RAI14 | SVCM | 8.51E−07 | NEXN | ACM | 0.003558541 |
| DDAH2 | SVCM | 1.43E−06 | NDUFB7 | ACM | 0.003806481 |
| GSN | SVCM | 1.53E−06 | SUCLA2 | ACM | 0.004286155 |
| RPL15 | SVCM | 1.63E−06 | NDUFB5 | ACM | 0.004799068 |
| SPTSSA | SVCM | 1.80E−06 | UQCR11 | ACM | 0.006452374 |
| PDIA4 | SVCM | 1.95E−06 | ABRA | ACM | 0.006860025 |
| CCT2 | SVCM | 2.04E−06 | NPPB | ACM | 0.010490844 |
| DBN1 | SVCM | 3.00E−06 | RAD23A | ACM | 0.01288501 |
| EFNB1 | SVCM | 3.20E−06 | HAGH | ACM | 0.020593587 |
| DUT | SVCM | 3.57E−06 | TRDN | ACM | 0.020960299 |
| CNPY2 | SVCM | 6.78E−06 | PDLIM5 | ACM | 0.021924528 |
| TSPAN3 | SVCM | 7.14E−06 | ALKBH7 | ACM | 0.027207251 |
| NME1 | SVCM | 9.05E−06 | GPX3 | ACM | 0.037085974 |
| NDN | SVCM | 1.02E−05 | | | |
| NPC2 | SVCM | 1.17E−05 | | | |
| HMGB1 | SVCM | 1.61E−05 | | | |
| SRSF11 | SVCM | 2.33E−05 | | | |
| HP1BP3 | SVCM | 3.68E−05 | | | |
| PHF14 | SVCM | 5.56E−05 | | | |
| MYL12B | SVCM | 7.85E−05 | | | |
| NACA | SVCM | 9.36E−05 | | | |
| PTBP2 | SVCM | 9.70E−05 | | | |
| TSPAN6 | SVCM | 0.000108403 | | | |
| MIDN | SVCM | 0.000142218 | | | |
| LMAN2 | SVCM | 0.000198106 | | | |
| COL18A1 | SVCM | 0.000286358 | | | |
| RAB34 | SVCM | 0.000288482 | | | |
| SSR2 | SVCM | 0.000449014 | | | |
| MEX3C | SVCM | 0.00055014 | | | |
| CCND3 | SVCM | 0.000551267 | | | |
| RPN2 | SVCM | 0.000553553 | | | |
| ALYREF | SVCM | 0.000652392 | | | |
| LIMD2 | SVCM | 0.000693598 | | | |
| BCL7C | SVCM | 0.000718712 | | | |
| YWHAQ | SVCM | 0.000978751 | | | |
| PPFIBP1 | SVCM | 0.001091885 | | | |
| RPL37 | SVCM | 0.001156565 | | | |
| ARL2BP | SVCM | 0.001266944 | | | |
| EIF4A1 | SVCM | 0.001323651 | | | |
| PFN2 | SVCM | 0.001341568 | | | |
| SMARCB1 | SVCM | 0.001375417 | | | |
| SFPQ | SVCM | 0.001586955 | | | |
| APEX1 | SVCM | 0.001989924 | | | |
| PRRC2C | SVCM | 0.002232754 | | | |
| SRP72 | SVCM | 0.002389675 | | | |
| RBM3 | SVCM | 0.002457787 | | | |
| NCBP2 | SVCM | 0.0025136 | | | |
| RBM39 | SVCM | 0.002686733 | | | |
| PPDPF | SVCM | 0.002883048 | | | |
| PALM | SVCM | 0.002958699 | | | |
| DYNLT1 | SVCM | 0.003167772 | | | |
| UBB | SVCM | 0.003450451 | | | |
| IGSF3 | SVCM | 0.004335328 | | | |
| RPS8 | SVCM | 0.004376366 | | | |
| ERH | SVCM | 0.004451114 | | | |
| CDK4 | SVCM | 0.004703628 | | | |
| UBE2V2 | SVCM | 0.004863735 | | | |
| FRMD4A | SVCM | 0.005365243 | | | |
| NHP2 | SVCM | 0.00561124 | | | |
| PLIN2 | SVCM | 0.006631859 | | | |
| SSR3 | SVCM | 0.007658092 | | | |
| SNRPB | SVCM | 0.007905315 | | | |
| DDX3X | SVCM | 0.008374448 | | | |
| MEX3A | SVCM | 0.010832848 | | | |
| SLC39A6 | SVCM | 0.013711442 | | | |
| SRSF10 | SVCM | 0.014963938 | | | |
| CCNL1 | SVCM | 0.016317268 | | | |
| HDAC1 | SVCM | 0.016649521 | | | |
| SERP1 | SVCM | 0.018149373 | | | |
| HSPA5 | SVCM | 0.018786636 | | | |
| TLN2 | SVCM | 0.020357464 | | | |
| MEF2C | SVCM | 0.021911837 | | | |
| RPL19 | SVCM | 0.022952859 | | | |
| RPS19 | SVCM | 0.025584362 | | | |
| NCL | SVCM | 0.029496025 | | | |
| TSEN34 | SVCM | 0.03200975 | | | |
| DYNC112 | SVCM | 0.03979637 | | | |
| JUND | SVCM | 0.040603337 | | | |
| CCNL2 | SVCM | 0.041091284 | | | |
| LUC7L | SVCM | 0.042662408 | | | |
| MTPN | SVCM | 0.046536579 | | | |

| gene | cell type | p_val_adj | gene | cell type | p_val_adj |
|---|---|---|---|---|---|
| EEF1B2 | LVCM | 2.88E−165 | NDUFA13 | RVCM | 8.23E−267 |
| RPL19 | LVCM | 2.41E−157 | PLN | RVCM | 7.71E−242 |
| RPS6 | LVCM | 1.20E−150 | PDLIM1 | RVCM | 2.69E−237 |
| EIF4EBP1 | LVCM | 1.02E−127 | COX6C | RVCM | 1.25E−190 |
| DOK4 | LVCM | 2.89E−121 | UBL5 | RVCM | 3.05E−183 |
| RPL5 | LVCM | 4.93E−117 | CRIP2 | RVCM | 8.15E−177 |
| RPL18 | LVCM | 2.30E−115 | FHL2 | RVCM | 2.42E−172 |
| VCAM1 | LVCM | 3.52E−109 | IRX1 | RVCM | 1.07E−158 |
| RPL12 | LVCM | 1.90E−108 | C1QTNF4 | RVCM | 3.33E−149 |
| HAND1 | LVCM | 8.84E−93 | DES | RVCM | 1.18E−145 |
| TBX20 | LVCM | 1.48E−85 | ATP1A1 | RVCM | 1.55E−145 |
| APEX1 | LVCM | 7.09E−81 | ATP1B1 | RVCM | 1.98E−133 |
| NPM1 | LVCM | 1.74E−78 | IRX2 | RVCM | 1.50E−132 |
| NME1 | LVCM | 3.87E−69 | ASPH | RVCM | 2.43E−129 |
| PPIC | LVCM | 3.19E−66 | NDUFB4 | RVCM | 2.07E−125 |
| FITM1 | LVCM | 2.30E−57 | BNIP3 | RVCM | 5.62E−124 |
| TMEM88 | LVCM | 1.58E−55 | UQCR10 | RVCM | 1.98E−122 |
| PYGB | LVCM | 1.69E−55 | NDUFC1 | RVCM | 1.30E−120 |
| PHGDH | LVCM | 5.52E−54 | COX7A1 | RVCM | 2.46E−118 |
| PPA1 | LVCM | 1.23E−52 | MEIS2 | RVCM | 2.13E−110 |
| POLE4 | LVCM | 1.63E−50 | COX6B1 | RVCM | 7.77E−109 |
| EFNB3 | LVCM | 1.35E−49 | EPHA4 | RVCM | 1.43E−104 |
| GPATCH4 | LVCM | 3.68E−49 | FXYD1 | RVCM | 3.59E−103 |
| SERBP1 | LVCM | 1.56E−47 | LDHA | RVCM | 4.84E−101 |
| MTUS2 | LVCM | 1.37E−46 | MYL3 | RVCM | 1.19E−97 |
| STK39 | LVCM | 2.02E−43 | ATP5MF | RVCM | 5.96E−97 |
| NME4 | LVCM | 9.10E−43 | CXCL12 | RVCM | 1.09E−96 |
| CCT3 | LVCM | 3.04E−42 | MYL2 | RVCM | 7.44E−87 |
| SRSF2 | LVCM | 9.35E−42 | PBX1 | RVCM | 1.76E−77 |
| AIMP2 | LVCM | 2.92E−41 | TGM2 | RVCM | 7.67E−77 |
| EIF3A | LVCM | 2.87E−38 | MTCH1 | RVCM | 1.48E−76 |
| TMEM97 | LVCM | 6.17E−37 | IGFBP5 | RVCM | 1.26E−72 |
| HMGB3 | LVCM | 1.29E−36 | COX6A2 | RVCM | 4.07E−72 |
| TKT | LVCM | 2.85E−35 | GAPDH | RVCM | 3.21E−68 |
| EZR | LVCM | 3.71E−35 | GABARAPL2 | RVCM | 3.63E−68 |
| TLN2 | LVCM | 7.86E−35 | TMSB4X | RVCM | 1.04E−66 |
| MARCKSL1 | LVCM | 8.69E−35 | FILIP1 | RVCM | 1.01E−64 |
| FRMD4B | LVCM | 9.12E−35 | PDLIM3 | RVCM | 5.65E−62 |
| SRSF3 | LVCM | 1.30E−34 | LMOD2 | RVCM | 1.01E−57 |
| SORBS2 | LVCM | 7.31E−34 | ATOX1 | RVCM | 1.47E−56 |
| FN1 | LVCM | 7.70E−33 | GPI | RVCM | 4.20E−56 |
| SLC3A2 | LVCM | 4.88E−32 | ITM2B | RVCM | 2.69E−48 |
| PRDX6 | LVCM | 1.02E−31 | UQCRB | RVCM | 2.66E−46 |
| BTF3 | LVCM | 1.32E−31 | TRDN | RVCM | 2.92E−46 |
| DNMT1 | LVCM | 1.85E−31 | MFGE8 | RVCM | 3.61E−44 |
| CCT2 | LVCM | 1.13E−30 | LTBP1 | RVCM | 1.09E−42 |
| ODC1 | LVCM | 5.79E−30 | TXLNB | RVCM | 2.12E−42 |
| LAP3 | LVCM | 1.16E−29 | BSG | RVCM | 1.69E−41 |
| SNX24 | LVCM | 1.74E−29 | NDUFA6 | RVCM | 6.77E−39 |
| SNRPD1 | LVCM | 2.16E−29 | CKB | RVCM | 3.01E−38 |
| ATIC | LVCM | 2.45E−29 | PDLIM7 | RVCM | 4.46E−36 |
| UCHL3 | LVCM | 3.38E−29 | TMEM59 | RVCM | 5.20E−34 |
| SFPQ | LVCM | 4.17E−29 | CTSB | RVCM | 8.92E−34 |
| C1QBP | LVCM | 6.16E−29 | WNK1 | RVCM | 8.55E−30 |
| SNRPA | LVCM | 1.14E−28 | RSPO3 | RVCM | 9.71E−30 |
| EMP2 | LVCM | 6.17E−28 | TSC22D3 | RVCM | 2.65E−28 |
| SNRNP40 | LVCM | 1.56E−27 | WDR1 | RVCM | 4.28E−28 |
| EIF1 | LVCM | 2.37E−27 | PDLIM5 | RVCM | 1.70E−27 |
| NOP58 | LVCM | 2.99E−27 | MYOZ1 | RVCM | 6.49E−27 |
| RBMX | LVCM | 6.06E−27 | S100A13 | RVCM | 6.04E−26 |
| CSE1L | LVCM | 8.46E−27 | PRKACA | RVCM | 1.94E−24 |
| ZC3H15 | LVCM | 3.50E−26 | RCAN1 | RVCM | 2.39E−24 |
| BAZ1A | LVCM | 3.84E−26 | PPP1R12A | RVCM | 9.25E−24 |
| LSM2 | LVCM | 6.40E−26 | TUBA1A | RVCM | 7.71E−23 |
| MTHFD2 | LVCM | 1.32E−25 | HMGN3 | RVCM | 9.14E−23 |
| TSFM | LVCM | 2.66E−25 | HRC | RVCM | 2.11E−22 |
| WNT5A | LVCM | 7.13E−25 | CNN1 | RVCM | 2.70E−22 |
| NUP37 | LVCM | 1.82E−24 | MYOZ2 | RVCM | 3.23E−22 |
| TCERG1 | LVCM | 3.27E−24 | LBH | RVCM | 5.57E−21 |
| WDR77 | LVCM | 7.68E−24 | PAIP2 | RVCM | 7.45E−21 |
| MYH10 | LVCM | 1.96E−23 | CCDC107 | RVCM | 5.98E−20 |
| SRM | LVCM | 3.38E−23 | DUSP10 | RVCM | 2.40E−19 |
| RPS12 | LVCM | 4.07E−23 | CYB5R3 | RVCM | 6.03E−18 |
| AKR1B1 | LVCM | 4.39E−23 | SLC6A6 | RVCM | 5.31E−17 |
| WDR12 | LVCM | 8.75E−23 | MAST4 | RVCM | 5.74E−17 |
| ME1 | LVCM | 9.01E−23 | MAP1LC3A | RVCM | 6.04E−17 |
| EIF4G1 | LVCM | 1.07E−22 | CAPNS1 | RVCM | 5.89E−16 |
| SRPK1 | LVCM | 1.11E−22 | DSP | RVCM | 7.99E−16 |
| GRB14 | LVCM | 3.51E−22 | MYH7 | RVCM | 8.54E−16 |
| MRPS15 | LVCM | 4.64E−22 | GABARAP | RVCM | 5.10E−15 |
| LTV1 | LVCM | 5.76E−22 | PKM | RVCM | 8.26E−15 |
| BVES | LVCM | 8.43E−22 | NDUFA7 | RVCM | 6.42E−14 |
| IGF2BP1 | LVCM | 1.77E−21 | HIPK2 | RVCM | 2.23E−13 |
| CDK6 | LVCM | 2.91E−21 | MTUS1 | RVCM | 6.99E−13 |
| POLR2G | LVCM | 6.46E−21 | ARL3 | RVCM | 1.19E−12 |
| EEF1E1 | LVCM | 1.18E−20 | ACTC1 | RVCM | 1.62E−12 |
| PSMG1 | LVCM | 1.39E−20 | TNNT2 | RVCM | 1.68E−12 |
| SF3B3 | LVCM | 2.16E−20 | YPEL3 | RVCM | 1.80E−12 |
| IPO5 | LVCM | 6.53E−20 | UBC | RVCM | 5.32E−12 |
| NAA15 | LVCM | 6.61E−20 | MYL4 | RVCM | 5.92E−12 |
| PNO1 | LVCM | 1.52E−19 | BTG2 | RVCM | 1.43E−11 |
| ABCE1 | LVCM | 1.85E−19 | TMEM50A | RVCM | 3.23E−10 |
| BYSL | LVCM | 3.44E−19 | TECR | RVCM | 2.63E−08 |
| FSCN1 | LVCM | 3.72E−19 | WBP2 | RVCM | 4.23E−08 |
| MRPS22 | LVCM | 4.00E−19 | ATP5PD | RVCM | 4.28E−08 |
| RSL1D1 | LVCM | 4.65E−19 | ACADVL | RVCM | 9.60E−08 |
| TP53 | LVCM | 6.50E−19 | FGF10 | RVCM | 4.22E−07 |
| TOMM70 | LVCM | 7.05E−19 | FBN2 | RVCM | 5.59E−07 |
| DDX21 | LVCM | 1.27E−18 | RABAC1 | RVCM | 1.65E−06 |
| PPP1R14B | LVCM | 1.38E−18 | ETFB | RVCM | 2.02E−06 |
| ITPA | LVCM | 1.55E−18 | BNIP3L | RVCM | 2.33E−06 |
| OLA1 | LVCM | 2.15E−18 | SSBP2 | RVCM | 2.91E−06 |
| MPP6 | LVCM | 2.28E−18 | AP1S2 | RVCM | 3.44E−06 |
| HSPA8 | LVCM | 7.42E−18 | TTC3 | RVCM | 5.06E−06 |
| POLR1D | LVCM | 1.62E−17 | LRRC10 | RVCM | 2.10E−05 |
| MRTO4 | LVCM | 1.64E−17 | BTG1 | RVCM | 2.44E−05 |
| LMNB1 | LVCM | 1.71E−17 | GRB10 | RVCM | 3.02E−05 |
| MAN1A2 | LVCM | 1.81E−17 | PLK2 | RVCM | 7.64E−05 |
| TMPO | LVCM | 2.92E−17 | CGNL1 | RVCM | 0.000451583 |
| EIF3G | LVCM | 3.92E−17 | RAB11A | RVCM | 0.000992302 |
| DCTPP1 | LVCM | 4.22E−17 | LMNA | RVCM | 0.001613405 |
| HMGB2 | LVCM | 4.39E−17 | PTOV1 | RVCM | 0.002043216 |
| NOLC1 | LVCM | 1.27E−16 | CHMP5 | RVCM | 0.002471188 |
| HSPH1 | LVCM | 1.41E−16 | MTSS1 | RVCM | 0.003362609 |
| PLA2G12A | LVCM | 1.62E−16 | RTN2 | RVCM | 0.008864217 |
| MRPL37 | LVCM | 1.94E−16 | SERINC1 | RVCM | 0.011700929 |
| RPS11 | LVCM | 2.18E−16 | HSPB2 | RVCM | 0.016119166 |
| GART | LVCM | 2.20E−16 | DUSP6 | RVCM | 0.02787035 |
| SRSF7 | LVCM | 2.33E−16 | PSAP | RVCM | 0.030468014 |
| RPS8 | LVCM | 3.28E−16 | | | |
| NOL11 | LVCM | 3.82E−16 | | | |
| HELLS | LVCM | 3.97E−16 | | | |
| RAN | LVCM | 6.76E−16 | | | |
| PPIL1 | LVCM | 6.81E−16 | | | |
| NOP14 | LVCM | 1.13E−15 | | | |
| GTPBP4 | LVCM | 2.28E−15 | | | |
| BAG2 | LVCM | 5.31E−15 | | | |
| SSRP1 | LVCM | 7.29E−15 | | | |
| IMP4 | LVCM | 1.41E−14 | | | |
| TOMM40 | LVCM | 1.44E−14 | | | |
| HNRNPAB | LVCM | 1.64E−14 | | | |
| WDR74 | LVCM | 2.40E−14 | | | |
| RPS16 | LVCM | 4.03E−14 | | | |
| WDR43 | LVCM | 4.67E−14 | | | |
| CTPS1 | LVCM | 4.99E−14 | | | |
| RBM25 | LVCM | 5.85E−14 | | | |
| CDV3 | LVCM | 6.85E−14 | | | |
| CKS2 | LVCM | 8.52E−14 | | | |
| APRT | LVCM | 8.58E−14 | | | |
| SDF2L1 | LVCM | 1.03E−13 | | | |
| NME6 | LVCM | 1.04E−13 | | | |
| YTHDF2 | LVCM | 2.02E−13 | | | |
| SNRPB | LVCM | 2.28E−13 | | | |
| FTSJ3 | LVCM | 2.42E−13 | | | |
| SAP30L | LVCM | 4.68E−13 | | | |
| MRPL12 | LVCM | 5.94E−13 | | | |
| EIF4A1 | LVCM | 7.24E−13 | | | |
| CKAP5 | LVCM | 9.82E−13 | | | |
| RRP9 | LVCM | 2.70E−12 | | | |
| PPID | LVCM | 2.91E−12 | | | |
| SRSF6 | LVCM | 6.10E−12 | | | |
| CCT6A | LVCM | 6.89E−12 | | | |
| CDH3 | LVCM | 9.64E−12 | | | |
| RANBP1 | LVCM | 1.05E−11 | | | |
| CCDC86 | LVCM | 1.27E−11 | | | |
| RRP1B | LVCM | 1.30E−11 | | | |
| HNRNPAO | LVCM | 1.69E−11 | | | |
| YARS2 | LVCM | 2.02E−11 | | | |
| SRSF1 | LVCM | 2.66E−11 | | | |
| C16orf91 | LVCM | 3.39E−11 | | | |
| FBLN1 | LVCM | 7.81E−11 | | | |
| RCC2 | LVCM | 1.10E−10 | | | |
| REXO2 | LVCM | 1.18E−10 | | | |
| SLC1A3 | LVCM | 1.77E−10 | | | |
| BOP1 | LVCM | 2.95E−10 | | | |
| MCM3 | LVCM | 3.07E−10 | | | |
| HNRNPD | LVCM | 3.23E−10 | | | |
| PPAT | LVCM | 3.41E−10 | | | |
| HDDC2 | LVCM | 4.81E−10 | | | |
| CDK4 | LVCM | 5.24E−10 | | | |
| BEX1 | LVCM | 7.07E−10 | | | |
| RUVBL2 | LVCM | 8.06E−10 | | | |
| PFDN4 | LVCM | 8.85E−10 | | | |
| SIGMAR1 | LVCM | 9.44E−10 | | | |
| SF3B4 | LVCM | 1.17E−09 | | | |
| GMFB | LVCM | 1.93E−09 | | | |
| CCNB1 | LVCM | 2.05E−09 | | | |
| MRPL19 | LVCM | 2.66E−09 | | | |
| TXNRD1 | LVCM | 3.24E−09 | | | |
| NUDCD2 | LVCM | 4.29E−09 | | | |
| TSR1 | LVCM | 5.56E−09 | | | |
| SEC61A1 | LVCM | 5.92E−09 | | | |
| DLST | LVCM | 6.21E−09 | | | |
| HSPD1 | LVCM | 6.53E−09 | | | |
| UNG | LVCM | 6.56E−09 | | | |
| DDX39A | LVCM | 6.79E−09 | | | |
| UBE21 | LVCM | 8.34E−09 | | | |
| MRPS26 | LVCM | 8.41E−09 | | | |
| EMC8 | LVCM | 9.10E−09 | | | |
| TYMS | LVCM | 9.50E−09 | | | |
| NOC2L | LVCM | 9.71E−09 | | | |
| THRAP3 | LVCM | 9.84E−09 | | | |
| GNL3 | LVCM | 1.04E−08 | | | |
| DNAJC11 | LVCM | 1.37E−08 | | | |
| DCTD | LVCM | 1.54E−08 | | | |
| HSPA4 | LVCM | 1.56E−08 | | | |
| RRAS2 | LVCM | 1.59E−08 | | | |
| STARD7 | LVCM | 2.12E−08 | | | |
| AURKA | LVCM | 2.38E−08 | | | |
| TARDBP | LVCM | 3.25E−08 | | | |
| PRTG | LVCM | 3.25E−08 | | | |
| DNAJC2 | LVCM | 3.75E−08 | | | |
| NUDT21 | LVCM | 4.55E−08 | | | |
| POLR3D | LVCM | 5.31E−08 | | | |
| CLINT1 | LVCM | 7.81E−08 | | | |
| EIF4A3 | LVCM | 1.05E−07 | | | |
| HDGF | LVCM | 1.06E−07 | | | |
| EBNA1BP2 | LVCM | 1.11E−07 | | | |
| OTUD4 | LVCM | 1.25E−07 | | | |
| MTAP | LVCM | 1.42E−07 | | | |
| MAGOHB | LVCM | 1.70E−07 | | | |
| FEN1 | LVCM | 2.19E−07 | | | |
| TRIM27 | LVCM | 2.36E−07 | | | |
| PHB | LVCM | 3.22E−07 | | | |
| CNOT1 | LVCM | 3.24E−07 | | | |
| DNAJC3 | LVCM | 3.59E−07 | | | |
| DCUN1D5 | LVCM | 3.67E−07 | | | |
| TBX5 | LVCM | 4.85E−07 | | | |
| FARSB | LVCM | 6.09E−07 | | | |
| WDR46 | LVCM | 7.42E−07 | | | |
| PRPF4B | LVCM | 9.04E−07 | | | |
| ATAD3A | LVCM | 1.25E−06 | | | |
| SMARCA5 | LVCM | 1.28E−06 | | | |
| PPM1G | LVCM | 2.28E−06 | | | |
| DEK | LVCM | 2.48E−06 | | | |
| TCOF1 | LVCM | 2.63E−06 | | | |
| UTP18 | LVCM | 2.64E−06 | | | |
| CAPRIN1 | LVCM | 3.69E−06 | | | |
| CHD4 | LVCM | 3.71E−06 | | | |
| CASP8AP2 | LVCM | 3.87E−06 | | | |
| EXOSC1 | LVCM | 4.75E−06 | | | |
| KPNB1 | LVCM | 6.22E−06 | | | |
| ANP32B | LVCM | 6.80E−06 | | | |
| RRP7A | LVCM | 6.95E−06 | | | |
| SYNCRIP | LVCM | 7.02E−06 | | | |
| RCOR2 | LVCM | 7.32E−06 | | | |
| GOT1 | LVCM | 1.38E−05 | | | |
| DDX46 | LVCM | 1.50E−05 | | | |
| DNAJC21 | LVCM | 2.20E−05 | | | |
| NAA25 | LVCM | 2.34E−05 | | | |
| DDA1 | LVCM | 3.72E−05 | | | |
| IWS1 | LVCM | 4.43E−05 | | | |
| KDELR2 | LVCM | 5.88E−05 | | | |
| MRPL15 | LVCM | 6.00E−05 | | | |
| PPIF | LVCM | 7.56E−05 | | | |
| PSMA4 | LVCM | 0.000121744 | | | |
| PABPC4 | LVCM | 0.000124615 | | | |
| SMARCC1 | LVCM | 0.00021718 | | | |
| ILF3 | LVCM | 0.000235316 | | | |
| MRPL47 | LVCM | 0.000438104 | | | |
| PTPN11 | LVCM | 0.000468729 | | | |
| FAM136A | LVCM | 0.000886127 | | | |
| PHB2 | LVCM | 0.001248031 | | | |
| GMNN | LVCM | 0.001894969 | | | |
| C1orf43 | LVCM | 0.002274767 | | | |
| TIMM9 | LVCM | 0.00288776 | | | |
| NOP56 | LVCM | 0.004193245 | | | |
| NCL | LVCM | 0.048031567 | | | |
| RPL22L1 | LVCM | 0.050574511 | | | |

1. A method of making first heart field (FHF) mesoderm cells, anterior second heart field (aSHF) mesoderm cells and/or posterior second heart field (pSHF) mesoderm cells, comprising:
culturing pluripotent stem cells (PSCs) in the presence of an appropriate amount of BMP4 and Activin A for about 1 to about 3 days;
thereby producing FHF mesoderm cells, aSHF mesoderm cells, and/or pSHF mesoderm cells,
wherein the FHF mesoderm cells are MESP1+, CXCR4−/low, GYPB+, CD1Dlow, TDGF1+, LHX1+, PITX2+, and GSC+,
wherein the aSHF mesoderm cells are MESP1+, CXCR4+, ALDH1A2-, CD1Dlow, PHLDA1+, PCDH19+, FOXC2+, TWIST1+, and FOXC1+, and
wherein the pSHF mesoderm cells are MESP1+, CXCR4−, ALDH1A2+, CD1Dhigh, HOXA1+, HOXB1+, HOTAIRM1+, TBX6+, and CDX2+.

2. FHF mesoderm cells, aSHF mesoderm cells and/or pSHF mesoderm cells made by the method of claim 1.

3. (canceled)

4. A method of making first heart field (FHF) mesoderm cells, comprising:
culturing pluripotent stem cells (PSCs) in the presence of an appropriate amount of BMP4 and Activin A for about 1 to about 3 days,
thereby producing FHF mesoderm cells, wherein the FHF mesoderm cells are MESP1+, CXCR4−/low, GYPB+, CD1Dlow, TDGF1+, LHX1+, PITX2+, and GSC+.

5. First heart field (FHF) mesoderm cells made by the method of claim 4.

6. A method of making first heart field (FHF) progenitor cells, comprising:
culturing the FHF mesoderm cells of claim 5 in the presence of an appropriate amount of IWP2 and VEGF for a period of about 1 to about 3 days,
thereby producing FHF progenitor cells, wherein the FHF progenitor cells are ALDH1A2-, HAND1, TBX5, HCN4, MYH6, LBH.

7. First heart field (FHF) progenitor cells made by the method of claim 6.

8. A method of making first heart field (FHF) cardiomyocytes, comprising:
culturing the FHF progenitor cells of claim 7 in base media for about 18 to about 22 days, thereby producing FHF cardiomyocytes, wherein the FHF cardiomyocytes comprise a first population of left ventricular cardiomyocytes (LVCMs) that are GJA1+, HAND1+, TMEM88+, and TBX5+ and a second population of atrioventricular canal cardiomyocytes (AVCCMs) that are BMP2+, TBX2+, RSPO3+, and MSX2+.

9. First heart field (FHF) cardiomyocytes made by the method of claim 8.

10. (canceled)

11. (canceled)

12. (canceled)

13. A method of making anterior second heart field (aSHF) mesoderm cells, comprising:
culturing pluripotent stem cells (PSCs) in the presence of an appropriate amount of BMP4 and Activin-A for about 1 to about 3 days,
thereby producing aSHF mesoderm cells, wherein the aSHF mesoderm cells are MESP1+, CXCR4+, ALDH1A2−, CD1Dlow, PHLDA1+, PCDH19+, FOXC2+, TWIST1+, and FOXC1+.

14. Anterior second heart field (aSHF) mesoderm cells made by the method of claim 13.

15. A method of making anterior second heart field (aSHF) progenitor cells, comprising:
culturing the aSHF mesoderm cells of claim 14 as an embryoid body (EB) or isolated day 4 CXCR+ ALDH− mesoderm cells in the presence of an appropriate amount of IWP2 and VEGF for about 1 to about 3 days (e.g., about 1 to about 2 days),
thereby producing aSHF progenitor cells, wherein the aSHF progenitor cells are ALDHIA2+, JAG1+, FGF10+, FGF8+, WNT5A+, and PHLDA1+.

16. Anterior second heart field (aSHF) progenitor cells made by the method of claim 15.

17. A method of making anterior second heart field (aSHF) cardiomyocytes, comprising:
culturing the aSHF progenitor cells of claim 16 or isolated day 4 CXCR+ ALDH− mesoderm cells in the presence of an appropriate amount of BMP4 and RA for about 3 days and then in backbone media for about 12 to about 15 days,
thereby making aSHF cardiomyocytes, wherein the aSHF cardiomyocytes comprise a first population of right ventricular cardiomyocytes (RVCMs) that are IRX1+, IRX2+, and NPPB+ and a second population of outflow tract cardiomyocytes (OFTCM) that are SEMA3C+, HAND2+, and FHL1+.

18. Anterior second heart field (aSHF) cardiomyocytes made by the method of claim 17.

19. (canceled)

20. (canceled)

21. A method of making posterior second heart field (pSHF) mesoderm cells, comprising:
culturing pluripotent stem cells (PSCs) in the presence of an appropriate amount of BMP4 and Activin A for about 1 to about 3 days,
thereby producing pSHF mesoderm cells, wherein the pSHF mesoderm cells are MESP1+, CXCR4−, ALDH1A2+, CD1Dhigh, HOXA1+, HOXB1+, HOTAIRM1+, TBX6+, and CDX2+.

22. Posterior second heart field (pSHF) mesoderm cells made by the method of claim 21.

23. A method of making posterior second heart field (pSHF) progenitor cells, comprising:
culturing the pSHF mesoderm cells of claim 8 or isolated day 4 CXCR4− ALDH+ mesoderm cells in the presence of an appropriate amount of IWP2, VEGF, and retinol for about 2 to about 4 days,
thereby producing pSHF progenitor cells, wherein the pSHF progenitor cells are ALDHIA2+, HOXB1+, HOTAIRM1+, NR2F2+, DUSP9+, and FOXF1+.

24. Posterior second heart field (pSHF) progenitor cells made by the method of claim 23.

25. A method of making posterior second heart field (pSHF) cardiomyocytes, comprising:
culturing the pSHF progenitor cells of claim 9 in the presence of an appropriate amount of retinol for about 2 to about 4 days followed by culturing in base media for about 12 to about 15 days,
thereby making pSHF cardiomyocytes, wherein the pSHF cardiomyocytes comprise a first population of atrial cardiomyocytes (ACMs) that are NKX2-5+, NR2F2+, and SCN5A+ and a second population of sinus venosus cardiomyocytes (SVCM) that are TBX18+ and SFRP5.

26. Posterior second heart field (pSHF) cardiomyocytes made by the method of claim 25.

27. (canceled)

28. (canceled)

29. (canceled)
